SEQUENCE LISTING (153 sequences)

SEQ ID NO: 1 - DNA, 32767 bp, H. sapeins
acacttggacacaggaaggggaacatcacacaccggggcctgttgtggggtggggggtgaggggagggatagcattaggggatatacctaatgctaaatgacgagttaatgggtacagcacaccaacatggcacatgtatacatatgtaacaaacctgctcgttgtgcacatgtaccctaaaacttaaagtataataataaaaaaaagatcattctaaaatttatacaagcccttagaacagttaaaaatatcttaccaaaagaagaataaagttggaggaatcactctacctaatataaagtcttactacatagctacagtaattatgacagtgttatattggcagagggataaatacatcaatggcacaaagaatagatagagaaactggaagtagacccaaaacaatatggttaactgacttacgaaaaaatttcagaagccattcaatcgaggaaggatagggtggtattgttgttttttgttttaacaaattgtgctggataaattggacatacctatggaaaaaaaaatgaagtttgacttaaacatcatactttacacaaatattaactcaaaatggagcatgggcataaatctaaaacttcaaactgtaaaacatttagaaaaaaataggaaaaaaatcatcaggatctagtgttagtggaagagttctaaatgtgatccataaaacaaaaacaaataaactggactacatcaaaactaaaaaattctactctgtgaaagacctaattaagaggacaaaagacaagctacaggctggagacaatatatttaatccacgtatctatgaaaggactcatatctagaatatataaacaaccttaagaatctgacagtaaaaaaaaaaaatcagactaactggaccactcatacattgctgatggaaatgtaaagtggtacagccattttggtaaacatcattgctctctgacaaagatacggtgggtcccactgatgaactgtgctgccacagtaaatgtagccactatgcctatctccattctgaagatgtgtcacttcctgtttcagactcaaatcagccacagtggcagaagcccacgaaatcagaggtgaaatttaataatgaccactgcccattctcttcacttgtcccaagaggccattggaaatagtccaaagacccattgagggagatggacattatttcccagaagtaaatacagctcagcttgtactttggtacaactaatcgaccttaccactttcacaatctgctagcaaaggttatgcagcgcgtgaacatgatcatggcagaatcaccaggcctcatcaccatctgccttttaggatatctactcagtgctgaatgtacaggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaaagcttttcatggattaggaaaaaatcattttgtctctatgtcaaacatcttggagttgatatttggggaaacacaatactcagttgagttccctaggggagaaaagcaagcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcagactgaaatgctgaaataacgataaaaaaaaatacagaggttaaactagcaaagtgagtaaagtcaagggataaagaaaatttgttggaaaactcacaaagcaggacataaagcaaggccattagatatatctcattagtgtgacatctgggaggacaaagcatccaaaccctttcttctatataagtggtgagatgatgaaggttgtaagagggcttctgcccccttgaagacttcagatgctggggaaaggatagataagaataaggatgaaccaggcttttggagcctgggaaataatgactagcgataaacctgaagggaagttaagtatacgatccccagataatactaaggagaaaggcaatgtgattctgcagccattgtagccagagataataagcccttgaggaaggggccaggggaatttttctaaggatagacagtattaatgcagcactctcttctgctattaaactctcattggcttctaaaaggagtttcggtgagtgatttgctgagatgttgcattttcatggctgctgcctttaggtattattgcaacagtttggaattttgaaattaaaacagttctgtaaaaccagtttagttttgtaaagtgtatgcatcaaagatgtccttcattcagacattactgagttacaactatggtgccaggtactgtgtcagggtactaggggtatggggataaaccagactccctctttgatctaaagcagcatgaggccaggtgagaggtttcaatataatgtgataaaatgtgcactaggtactaagggatcatagagaaaggaacacattaaatggggaaacaattgatagagagagaatattttcatctgggtcttaaaagatgagtaggcgttctctctctttaaatgtctgatataagggcattttatgcaaagaggatcactcgtgcaaagactcagatttgcaagaacgtgaggtatttcaggagttttgtatggttccatatggactatgacaagtgagacaggtaaactaggcagagctggtcatcagataatgaagtcattaacctaaggagattggacaataaaatgcaatatggaggtatcgaagtataaacataaggagtaccactgatggctgatttaggatgcccagtctggcaacacgctaatgaaatgatagtgggggagggggccgtaccaagactaggagagagcagtcctgagactattgcaattatctgcgggagacataaaggctagaacctgaacagtagcagtacaaaaaaagaggggagttcaaatgatattaaggaagtagaagtggtatgacttaaccatctgggtatggaaggggaaatggctagagtcttggggactttgtgtttgatgtgattatggaccacagaataatgtctaagagaactggctctttagtctgactgccagagtctgaatcctgaatgttttagtatgttaccttgggcaaagcccttagcctctatgaatctatcttcctcattcataaaaataagatgacagtgcctatctcgtgggacttttgtgaggatgaagtgagataatggatgcaaagttactgagcacagtgtccaacacagcagaagcattacatatacattagctattactggctacattatgatatacagttagggagttggaaagataatctgaaattcaggagacgtatctgactataggtgagtatttggaactcattgttctgtaaacagtagttacagccacgtgtgtgggcatctggagagtgagcatggatattgtgatacctagtacagtgcctggcagtagtggttgtatgctcagtaaattttgttgacagggtcagggccggactagactgtggtaagcaaggcctgtagggcataaatatacttgtatgccccgagaagtgaggacctcttaaatattgtgccctacatgccttgtttggttcactcttgtcccagccctagcaagtatatataaggtgaaaaaggaaaaagctgaggctggagcctgggagaaccctggacatttaagggccatggagaggaacaggagttaatcaattcaagtgctggatggataacaggagttagagcaaagcggggaaccagaatagagtgattattataaaaagagtttcctaaaaagggagagatcaacaattagaaattatttagagcagccagtaaatacataaactcaaaattattctttaggtcattcctgattgtgacaatagtcatttcattatataaatgtgattaaggaaggaaaagagctacacagaagttattaaagagctaaagagaattgagaaatttaaaacagaagaaagtagggccaacatgaaaggagtagggagaaaaagagataaccagcatattgtctcactgatcctgccaacacctgtgagatagatattcttattatactactgttaaacctaatttacatttgacaaagttaaggttcagagcttgtgtgacttgtccaaggtcacaggtctagaggaggcagatacttgattcaaacctatttctgtctgatctgattctaaagtctgttttttcactcaaccacactgtacagtcagctctccttgtgagttccacagccacagattcaattaactgcagatcaaaaatattcaagaaaaaaatggatggttgcatctctactgaacatgtacagactcttttatctttcattattccctaaacaatacagcataacaactatttacatagcatttacattgtattagctattaagagaaacctagagatgatttaaagtacaaaggaggatgtgtttaggttatatgcaaatagtaagccatttttatatcggagacttgagcatccacagatcttgatatttgcagggggtcttgccaccaattttccatggatactgaggaacgactgtaaatggatgcaggcatggatgctatttaggagtgtccagggccaagtaaatgagttgctgagcagagaggtgggtggaggctgtgaggcatcaatatgtggtggcatccatctgcattttggtgatttttttccttcacagtcctcggctgtctgggaagagaaggatgaaggcagatggctgctccaatttaggggctaggattgcagggtgggcacagcattgcaaacgagtgaaggaaattgagaaatatggccaatgaagagttgaagagaggcctggcatggtggctcacacctataatcccagcactttcagaggcccaggcaggcagatcacttgaggtcaggagttcgacaccagcctggccaacaaggtgaaatggtgaaaccccggctttactaaaaatacaaaaattagctgggcatggtggcgggtgcctgtaatcccagctacttgggaggctgaggcaggagaatagcttgaacctgggagatggaggttgcagtgagctgagatcgcaccactgcactccagcctgggcgacagagcaagactctgtcaaaaaaaaaagagttgaagagaaaaagtctaggctaaattcaaagaaaaaaagtgagcccaaaaggaacttgcagagcaagggaaaagcagggatgtcaagggactagaacactccataaagtgaacagctgcaatgaaaataagggaagaaagtttagttcatctccgtttctttcctttcctttttactttcctttctcttcctttttggagttaatcaggaagtagtcccaaataccccagaaagttcatcttataagcccttggtcctcttgagatggtatcagatatattgctagacccttgaagaaaggaacaactccaggcaacttcttgagtccctgttattaattttatacatacacacacatatatgtatatacatgaaaacacacaaacacatgtgtgtgtatacagccatgcattccttaacaatggggatatattctgagaaatgtgtcattaagcaatttcatcattgtgcgaacataatagagtgtacttacctaaacctaaatggtatagcttactacatacctaggttgtattgatgtggcctattgctcctaggctcctgggctgcaaacctgtacagcatgtgactgtactgaacactgtaggcaatggtaacagtggtatttgtgtatctaaacatagaaaaggtacagtgaaaatacagtattataaccttatgggaccactgtcgtataatgtggtccatcattgaccaaaatgtcattgtgcagcaaatgattatctcatatatatatatatatgatatgatatatatgatatatatgtgtgtgtatatatgtatacatatatatgtgtacatatatgtatacatatatacacacacatatatatgtacacacatatatgtatatatatgtacacacatatatgtatatatatatgtacacacacacacatagagagagagagagaggagaggagaggaaggagggagggaaggagaaatatgattcagatagagacatctatcctccagagttcaggagtgtctcttcagactaggtagatgtagcttaaaaaaaacatatcctgaattctagagagatgcttaaatcactgcaattcctataacacttgccaaccaaaggtgctgttgatctgaaattgcttttttaaattaatgcagtgatttttctttaacatctagtgacagacactggggtcacatttgcagctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacagtgagtatttccacataatacccttcagatgcagagcatagaatagaaaatctttaaaaagacacttctctttaaaattttaaagcatccatatatatttatgtatgttaaatgttataaaagataggaaatcaataccaaaacactttagatattaccgttaatttgtcttcttttattctttatagactgaattttggaagcagtatgttggtaagcaattcattttatcctctagctaatatatgaaacatatgagaattatgtgggttttttctctgcataaatagataatatattaaactttgtcaaaaggactcagaaagatcagtccaaccctctaacccatattgaatggtgatatactacagggttatgccagtgtgggaactatcgctggtaaataagtttaatcctccctagggcttcacaaagaacattgttccaccccaggagggtggaaggaagaaactgaaatgattgtgtcttagaacctaatgaaagtttgcattcctcagtaaaatcagagactgctgattgacttaaatgtttatagcttcaaagtcctcctcattatcatggcccacaagcccttccatgattgtccttccccaccctccccattacccttcttgcctcctctgctacttctctcctcgcacactgggctccagccaccctggccttcctgtcacttcttgcacactctaggaatgctcccactttggaggctttatctggctgtttctcttatttggctgttcccaacttcctgtgggctgactccctcacctccttcgggtctttgcccaaatgttaccatcttaatgaggcctaccttcaccatctattaatacttcaacctgccccagtagccttaccactctagacacctgtacagaactccactctactttttaacagagcttttcaccatctaatgtatcatataatttcttactcatactatttatcatttattttctcctactccactaaaatgcaagtttcatgttggcagggatattcaattgttttgtttattgatatattcctagcacctagaacagtatctggaaaagaggtactcagtaaatatttatcaaatgaattgaccaaaagaaggaaaactcaaaactttaatgacaactaactttaaagctacaataacttaaaattcagcagtaggattttgagggagggtaagtttcaaagattgacttacctaagactatctgcataaataaaaagaaattaatccagacaacaaattcaccaacttccatcaattggaaatccaattctattttctacagtttatgttctggagacactactggacactcttttactctcataactcataactcctccacttttgtttttaaatcatgagagaaaaagagttgactctgttatattgttttatctacctttccttgatcttagaaacgaatactaccataccagcttctactgaggtgccccctaaagttagtccaaataggtctttgcaatctccattcccgcagaatttagaactttgaatcacatgatttatttctaaaagtaaatccatgccgattttccccaccaaaaaattcctgactattaaactcctacaatcccttcattgctcactccccacccccaggatcatattttaaagttgggcccttgccttttgggtcacataggtacactgtttgctataccacaggtatagctatctggaaaacatggagggtattattctgttactactgcttcgtcaaccaaaaaataaaacaaaacaagaacaaaaaagaaacaaactccctgcctcttttcacttgcagtcaaggttcctaaccactacaaaattagcctatgtttcttcttgcacatagtagaaacccaagcttctcactgctgtgctattctgtaccatcaactcatcacataaagagcctggttgaagaatgattgtccaaccacattactagcatctgtcaagactttccagtttacaaaaggcctatcacatttaaccctcacaccatccttgtgaccaaagcattattaactccattttacaggagagtaaactgaagcttagggaagttaaaagaactgccaaaggtctcccagttggggagtcatgaagcccagaagagaagccaaattctctgctgctcaaccccttgctttcactattacacctcagggccttcaaatctaaatgcagttattcattaaacaggaacctggtagtcttaaacaggaatctctcacttggtaagatcttgtctcttgttgtatttgaccccaactgtctatggctttgcctgaacccaaagtacacacagcctagaaaccaaaggagaaccaaatgtgggataaaatgacactcattttaacgacatgtctcagcaaatgagttcctgtgtagctggctgaaagcccagaccctttcagtaaaacatcctgaataattcacatttgttggtctataatataaagggcaaatgtagctcatttttagaccagttctgaacatcaatagtaacaaaccagagataaccgattttgttttcatagaattggaacaaattagagtatctgtgcaaaagcatatcagatctaggagcagaggggacaaggtctaatttttaaataagcaaattttccagggagggactacttatgataaagggatattagtctcttagtcaacggaacctggatacacgcttctgacagagaagagggagaataggcaggaatctacacaccagatgtcaaggagatttgctttaaaatacgactgataattagaaatttctcagtttcccccttttccctcattctttgattcttattgttatctttatctcttactcctttgtttctcatatattgagtcttacagatcaagctcctatttttttcttcaggggtatttttctagttcaaagtgcctaccatctcccttctggttctattcatccttctctcccaaagctcctttagaagtgtggattaaggcagagcactaagaaaccagacttaaagattcccttctcattctgacttttctcctttcacctattccttcctcctgttttcttaccatcagtgtcttcaaaggctttcaagtacacggtaaatgcagaaacttcaagaaaggcagaatggaaacataaccaatgcatacataaataaagcacactgtagaatctttttaaattctgtatgatatatcgaatgctgtctctcacattacctagaccatttgaaaccgaatttgtaaaacatagactatctttaagtagtaacagatgcttctgacatgttttctattgtcttgaaccattactgcatatgatacatcaaagttaagtgacaatacaagaaagcagattcatttgctccctgcctaggccgtcagttcctaaagtggaaacgccatatattatctagctcagtttgctctacaagacctgcaatagagccttgtgtgacatagagataatatttgttgaagcaattaaatttgacttggaattaactctgccatcattctataaggaaggattgaaaatccttctcaccctgtgctgatatagtacctttctatacaaaaacgtccttctccctcttcccttggattgcataaactatgtacatgccttcctcagggcacttttctaggacagtgtcagcctaaggatctttgtttgggtggcttttagaaactcaggaagacaggagcatcatatgcctataggcagctggcttccaggtcagtagttttgctctgaccctaaaatcagactcccatcccaatgagtatctacaggggaggaccgggcattctaagcagtttacgtgccaattcaatttcttaacctatctcaaagatggagatcagtgtgagtccaatccatgtttaaatggcggcagttgcaaggatgacattaattcctatgaatgttggtgtccctttggatttgaaggaaagaactgtgaattaggtaagtaactattttttgaatactcatggttcaaagtttccctctgaaacaagttgaaactggaaaatgcaatattggtgtatcataatttttcttaaaaacatacctttgatgcttataaacatttcatttgtagtgatagttttcaggatatgagttcaagaagctacattaaaatcaataacaatatttggtaactaatattaagtaataatgatgttccactcacttattaatctttaatacaaccgtatgtggttagtactatcattatgcgcattctatgcagatgagaaaaccgcaactccaacggccaaaaattacagaggcataaatggtttagacaggacttaaacttcagtgtgaccaaaacccatgcttctaactactatattcaaaactcagagaaaactgaacccagaaaattgaaatcatgactaaattgctatcaacataggtagtgaaagtcaattaagtacagaactggagtatgactggccaattatcccatataatgggaattctccacatgtacaaaccacttcatatgctaaacttgttgacaacattcaaagctcatccctgaatttgactatattgattacatcaaaaatgttacatagcaaccttagaatccttgtgtaccttttcttctcaaagcctagattatttctttttccgacgtttcagtaattggagcagtaaaccccagtgtcccttacctacttgtttattacctccagatgcaatattactggtactgtgattgagaaacgcacacagtgctaatgaggaattcactttctactctgacactctggaagaatagagatgcaatcctaaggaagaatttaacaccacaggctacatgactaaggataaagagtagaaaattagcaggactctattaaggcattacagcaatccacctgacagatgaaaaaggcatgaaatgaaatgaaatgtagcagctacactcgtcctattgagaaaggaaaaaagtcacctgtaatgttgttcagaaatcctttcagtactaaaaaattcattgaccatcttcctttagtctcgaaaatttcttagaaggtaaaaaaaggaaaaggtgacagggcaaagacatttgaaaagaaagaaaagagtgaatgaacttgcacacctggcttggactcccattcccttaggtttccattgtgggggacaaactaatgcctgggttacctttcttgagagtgtgttaattgattcaatatctctgaagtgctactttcatctgaaaggttataatttgaaattcagatttacctggataaatttgatcttgctattatggaaacctctagaaatccttggagtagttactcattatcagcttaaataatatagccggtggagctgagggaatgagtaactcaattagtctcagttacaactgaagggcacattgttgtaaactataattgaaaacataaatatctttacctagtttaaaaataaagatgctttaaaaggaggaagggaatagccctgaggaatgtaaatataagcacaaaacttctacaacagagtttgctacgtgtgtggctgtgttccacccagcaaaaatgctaagtctacaactgacacaacttggatactctcatgttcccacattttggtttggtcaaggctgtgcagttgtactgcagccaccaccactcctggcctctacagtatattgatctgacccaccaatctgatcaaggtttagaaaaatattttcagcccagttagctcacaaacaaaatgagaattcccacaaattgctctttatctcagacaacagaggaaagctacagcaaaagcataaacaaattaccatttaagtttgttgcttcaaattaaagactaattgcaacagctactagatagcacagtttatggggcatctcggccccaagtcttttgtcttataaggtcttgaaaaaaagaaaggagattttcatcaataagagttttttgttatctttttcccttgttcatcaggcccttcactgcgagagagaggtgtaaacgttcagggcatgcattctagttaaagaatattaattggctattgggtccctttggttagaataaagacctctgtatgatgtccctagctgtacatcaaacccaaatatctctcagataaatgaaggtctgtaagaatttggtcattcctgtctcttctaaagagtaacagaggcattttcccgcagtaaagtagaatggaaagaaaacaaaaatcacaagcctataaacaccttcttcaattttcccagcatgtcacagacactactgtcttatttactacgtatttctgaggagtaaaaaaaggaaatatgttgagtttagctgaagcacagcatattttgtggtaaacttgttaaataaaacatcttttgtccaagctttggttgtcacacaagtggatatatcaggaaatataaaggcagaataaactaaagcagaacatactaacatttgtagtaggcatgaagggaattagaaagtgtttgtgttaacatggagggagggagaacagatgctttgagatgttcttcaacagatattctaggcactgagacccccttcgggaccagagagagcccatatccaccacagtacctgacacataaatgctcagtaattgataaatgagtcccattctaactgttccttagccctgctctatggaactctcccctggaattccttgtgccattattttatttctggaatcttcagccttttagctgagggcaaaagattgctgattaggaagcaatatttcccacctcctgcgcaaaacaagccaaagatcaacagcagcagcaacatactgagccctaaagggcaatgacaaatgtggagaatgatacagaggtctggttacttcttagccaatgacacagaatcacaattgagaaaacacagagtttattcattcccattgtgcatgccctggacaaaccaagctgcacctttcgtaacttatcacaatctcatattgacggaacactttctacaggtaatgtttgatttggctgaacactttagcattgcttcgtagcaacaaaatgatagctagtaacagaaaaagatccagggatattaccactgttagtgaggagaaaggccttttaattaattaattaattaattaataggaccaagtgccatctttttggatcatgcccttagtggattattggtagcaaaggttaaagctcaagctggttcctttgtccccctggcaacagttgatttgcctccctttatctcctgaagtatccgtaaggactaagagccaattattacatttggctatgctagcatatgtaaaatagagtttaaaagtttagattcatcactcaaaaattcatattctccaaaaccatacagtcactctgttagcctgtgttcccccagaaaaaaagtcacaagcttatttattaacatgtgcaatcccagggggcaagagaaaggaactgaagagtgaggcagaaaggaaaagaaagccaataagaggatgagttatcaaactactcgtttctaacagcaactgattgcttaacttcctgggactgtctccaataagtcaaattggcctcaggttagtccacctgagtgggaagaagcggtgaaagaatttgtctgtcagtatctgtctctcattggttagaagttcgacttatggggaattaactccctcacatttcctagttggatatgcttgggtacccagagggtcatatggcatccatgcctcagcatgaacagggaagcttcaaggcaaaagacacatagtgcagctatgagccaaggcaattcaaggatacacccataggaggctggttgacatccacccagagctaatcaccaccatgctggaaaaagacacaggtgaagctgagaagaatgaaggtggtgcataggaggtatctaatacagtcactcattttcaaactttccatgttatgattgcactgaccactgaggatttctattgaaagttttactgttgtcaaacacgtacacaaggggaaaggtgtcttacattgtttatgttcctgtgctgctctagaaacagaaataggctcaagagcagagcctgtttttcttaattcagcaggtctaagctaacaagtcctgaaacatggtacttcctgttattggtattgcataggagaaacaaagggaaagcacagtaattagaaaatacaaaacaagatggcaggaataagccaaaaatatcaggaaacacaattattgtgaattgggattaaactaatctattaataatgacaactttcagcttggagttaaaaatttaattgtatactgttaacgaaagtgatacctaaaataaaattacactgggaggccaaaatgaagggatgtgaaaagaactatcaggtaaaaactaacaaaaagaaactagcaaagcaatcttaatatcagacaaaatagaatccaagaggaaaatcatttcaaaagacaagagattttttttattaataaggggaattgcataggagagtaaagaaaatgtgggccactggaatgcttagcactaatgacatattggtctttggtcttcagttaccttacaggaccctatttcattctcttatgtttgatatgtaaccacctcagccagcttcaagttgctttttggccctaatggacttcctagcactataatttcttttttttaaatgttttattttaggtttaggggtacatgtgaaggtttgttacatagataaacatgtgtcacaggggtttgttgtacatattattacatgacgcagatattcagctcagtaccaaatagtgatcttttctgctcctctgcctcatcccaccctcctccctcaagtagactccagtatctgttgtttccttctttgtgtttataagttcttaacacttagctcccgcttacaagtgagaacctgcagtatttgatttttgttcctacgctagtttcctaaggatgatagcctccagctccattcatattcccacaaaagacataatctccttcttttctatggctgcataatattccatggtatatatgaaccacattttctttatccggtctgtcattgatgggcatttaggttgattccatgtctgctattctaacactgtaatttctaaagacttccagattctacttttataggtaacctgttaaacagtctagctctggaagccaagcaatttctagaataactaagcaatagaaattacacttcaatgcagaaaggcagtatctacatgagattatgaaattgcggttgctttttgtgttcactgaaaaaaataagtaaaactgtaactttcagaaaaaatgattgtacatatagaaaacccaaagcatctaaacaattaaaataaataagtatagaaagattactggatacagagtcaacatacaaatatcaattgtatgtctatataccagcaacgattcaaaaatgatttttataatagcattaaaaattagacgcttagtaataaatgtgagaaagatgtgcaagaactctacataaaaaattatgagacgttattgagaaaaattaaggaaaacctaaataaatgaatgaataggcaatgtttatcaattaaaggatacaatatagtaaatatatcaaatgtttactaatggattcaatgcaataccaaagtgcccagcaggctttttttggtggtgggaggtcgggcaggattcataagctaattataaaatgcatatggaaatgcaaagagccaaggatagccaagacagttttgaggaagaataaacttgtactacttacactaccagatgtcaagacttattatcgagttacatttattaagacagtgtggtactgacacaaggatagacaaatagatcagtgaaacacactagagtgctcagaagcacacctgtacatatataaaggcttgatttatgatagaggtgccagtgcagtagagaaggaaattattggtgttttcaataaaaagtgataggtcaattagatattcatatggcatgaagtatgaaacaataacaatttatattcataacttgcagaaagcaaaaatttcttaaaatacaaaaagtgatcaccataaaggaaaagattgataaactggactatattaaaactaaggactcctgttcagcaaaagacactacttcgactgaaaagacaagtcacagagtgagacaagatatctgcaatacagatacctaataactgaaccccatacagtgatggtgggaatttaagttcgtacaatcattttagaaaattgcttggcagtatctactagatctgaacatgtgatccagtaattacactcataattataagccagtaaaaaggcatgtttatgtcaccaaaagatatatacaagaatgttcattacactattatacataagagccaaaaactggaaacaaaccaaatatccattaacagtagaatgaataaataaaagctgtaatagtaatacagtggaatactacacagcaatgtaaatgaactactgctgtacaaaacaacatggtttaatctcacagacaaaatgttaaatgaaagacacagacgagtacatattgcgaacttctgtttataattcaagaactggcaagaactgtttactgtgttagaagtccaggtaatggtaacctataaaaaggaaaaagggtggaatgattgggagggggcatcttctggggtattgataatgtgctatgtattggtcagtttagtgtttaaacaggctcatttactttgtgaaaacttacactaaaattgtgtgtattttttgaatatatgttatacattaataaatagggtttttaaacctgtagttcataatttagtgaaagtagaatatccaaacatttagttttaaaccaatcaattatagtgctaccatcatttttatgcattattgagaagtttattttacctttctttccactcttatttcaaggctccaaaatttctctccccaacgtatattgggggcaacatgaatgcccccaatgtatatttgacccatacatgagtcagtagttccatgtactttttagaaatgcatgttaaatgatgctgttactgtctattttgcttcttttagatgtaacatgtaacattaagaatggcagatgcgagcagttttgtaaaaatagtgctgataacaaggtggtttgctcctgtactgagggatatcgacttgcagaaaaccagaagtcctgtgaaccagcaggtcataatctgaataagattttttaaagaaaatctgtatctgaaacttcagcattttaacaaacctacataattttaattcctacttgaatctgcttccttttgaaatcatagaaaatatcagtagcttgaattagaccaattaattttctagattgcatcatattttaaatataaactatgtaatcatctacaacctgaattctttctgtgtccaatttgtccaatttttttctctaacatttatatcacaaagcaattaatttgtgtgatttctgcatatgtatttgtaattcatcaagtcaaatcaatgtagtaatactatatcataaaatatacacaaataattgagtgataggcttctagtataaggacggtaagtttgaagcatgattctatctgggctggctagtttactctgagaaagttattttttattgttgggtcttaagctgagtttacacacttggtgtcagaatgattccggcaatgaactgttttatgttctgctaggctgatcagcacaatctatatggctgtgaacaaaacaatgtttcccagtcatgccaaccatgccaccattttaacagctgattagtgtattcagaacatctccactccatgttcgtatggctgttatctaaagatgaaagcagtagacacttttattttttgaaaaatttaggctctgcagggtcaattatatttgataaatgaggggcttttttgaagcaaactagatataatttcttttgcatttctaaagcctgatatcttattaattggtacattaaattgtgcaccatttctctgtaactgtttcagtacctgtctcagcactataccaggcagaagaaattaaagaaagaaccagtgccgagatcagcttggtcagggagaccctaatcctgcggcactagaggaattaaagacacacacacagaaatatagagtatggagtgggaaatcaggggtctcacagccttcagagctgagagccccgaacagagatttacccacatatttattgacagcaagccagtcataagatttactgaaagtattccttatgggaaataaagggatgagtctggctagttatctgcagcaggaacatgtccttaaggcacaaatcacttatgcaattgtctgtggtttaagaacacctttaagcagttttccgccctgggtgggccaggtgttccttgccctcattctggtaaacccacaaccttccagtgtggatatcaaggccatcacgagcatatcacagtgctgcagagattttgtttatggccagttttggggccagtttatggccagatttggaggcctgttcccaacaaaccagaagctaggaatatatatcctgcaaataaaatgaagaatctctaaggcttcagggcctgcccacttgttcttctgcctggttcttcacatacactgtctcaaagctagtctaccttgagaggagcatgaatatgtgtgtgggtgtgtgtctgtgtattttaaccttaaaaacctaacttccagtatagacagatggcatactagctaaacccttacaagttcttctatgctataaaagagaaacagaattgagaaccacctccaactattaagtgttatatttgaatatagccttagctttagcagaataagtaggccaaacttaaaataagcttttctgccttttcaatgataaaggtcccttttctgtagccattgttgattgtgtacacttatacataagtattttgaactaatttcctgttttctcaaccacttgctgtcttcatgatactttgtcgcagctggttgctatagaaatgtctgttacaaggaatgtggcttgaaggaaagtgataaatgaaaatgaaatgtgaagtgactttgtttgactacaaattcccattctggtagtccccagtgtatcaatacattatttttctttagaaaataaaccaacccaaggaaaaatggtgggcaggtcctggtgaatatggctgtgataattatattagcaatctctttggctaatatttgaagcccaaataattgaatcacaatgatctctccccagaaaatatataaaatgcaccttggaatctagaaggccttttagtctgcaaaagaaaccttcttaatcataagcagcagaagtcccatttaccaaattggaaagttaaagttacaaagcatcaatcataagacttccattcagggatggcaattgggagtaagactttttagtaaagaaactaaacacaaagtcattagactctgtaaaagtcttaccaaatttgattctggaacacctattctatttccgtaaagatgatgaattccggagccaaatgttcttttcatgaaggatttgaaaactgtccatgaaaataacgcaatcaaccttttagcttgagactctattcactgattagatttttttaaatactgatgggcctgcttctcagaagtgacaaggatgggcctcaatctcaatttttgtaatacatgttccatttgccaatgagaaatatcaggttactaatttttcttctatttttctagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggtactttatactgatggtgtgtcaaaactggagctcagctggcaagacacaggccaggtgggagactgaggctattttactagacagacctattgggatgtgagaagtatttaggcaagtttcagcactaaccaatgtgagaaggcctccagagatgagcagttggtgaaagagaggctcaaaaccagctaccatacaggtcaagaagaatttggcattaaggaaacagcatagcaggattccagacaggcaactggtcaacaacatgaaggtctggaagaaaggtcgcaggtactcaggttcagggcactacttcagcttcagcccttgcaaaaactggtgagagttggaaagtctttaggctaagaaaaattggattatttaaaagggggtaaagaaagggactcaaggaggaaggattaaggcaagaactaggttccaagaaacagggcatgagagagagtcttgatctaccactatagttctcgtggtagcatcagaatcacctgggaacgtagaaatgcaaattctcctgctctacactagacctaccaaatcagaatatctagggggtggggcccagcagtctgtgcgcaaacaagcactgcaggtgattttgatgcacattatagtttgaaaactaggccaggtgcagtggctcatgccaataatcccagcactttgggagactgagacgggaggattgcttaaacccaggagtttgagaccagcctgggcaacacggcgaaaccccacctctaattaaaaaaaatacaaaaattagctaggtgtgatggctcccacctgtgctcccagctattcaggaggctgaggtgggagaatcacctgagcctggaaagtcgaggctgcagtgaattgtgatcacaccactgcacttcagcctgagtgacagagtaagaccctatctcaaaaaacagaaaaagaaaaacactggcccaaaggaaatgaacttgttacagaagccggggttcaaaacaccaaataatgcacttgtacctagtccttcccgggtgctctgcagacatttctccaagcgtagtctgcaaacaacctacatatgtagaattacctatgcacatttttcatttaacaaccaagagctacatttgtagcaaaatctgggttgtaacttagcctacagctgaagcctaagagattccgtctgtgagaagaaataacccacctctttggcccccctccccaggcaggaagccaggatggtccttatataaagttgtgctgtccaataggtaaccactagccacatatggctatttaaatttaaattaactacaattaagagaaattaaaaattcaattcctcaattgcacctgccaaattttaagcacataacaaccacatgtggctagtaactactgtattggagagtgcaagcggagatagaacactctattactgcagaaatttctattggatagcacttataatagtttagtgtaacttaaaactccctagttgccacagtcatgatttagtagtaatttcatggatttctctactgaggttagaatctctgccattagagactgataaatttaaagtttgcaattatcaaactggtgacaatttaagccagaatcaggtaaatgtcctcagttttaacagcattggaattttctgggactagctgtgtatctatccaggattcttgagaatgcctgccatttttcaacataatggatgtaaggtattacacatatacctggggatggggtggtaggtataattgcacaagcattgtggagaatggtatcaaagagtggcagaacatcacaatcaaggttttccctttcttttacctttgctttttaaaaagacaatatttgctggacctgatcttataactcataaatgggacactgtatgttcctttttacctcctctgtttctacttaattgcaccctatgaggactgcttcccttacctaccataaccccttccttcactcatccatatctttactcttcttcacaactctgtaatattgaccttctttatgaacctttcctggaacaatccctcttaagtgcaagcactgttattatgccttcaatgtatttaatatccatgtatctattctctctaattttgtcattttgtgttctcatgtattttcattcattatgtgtccaacttccatggataacatggttacaacaaaagatcctactttatgacaattatcttccttgggtttgtgggacatagaacagtgctcagagtaggggatccaagaacccaggagaatatattagctaagaagataacttccgtttttaaaagtccaagattcaggagatcaaaaccatcctggctaacatagtgaaaccccgtctcttccaaaaatacaaaaaattagcccggcgtggtggcaggcgcctatagtcccagctacacgggaggctgaggcaggagaatggcgtgaaccggggaggcggagctggcagtgagccgagatcccgccactgcactccagcctgggcgacagagcgagactccgtctcaaaaaaaaaaaaaaaaaaaaagtccaagattaaaaaaaaaaaaaaaaaaggatgtctgctttgtgagtttagcattgtctccttgtcattccagaaatgaaatggcaaatacatttaaatcagaactaaaaaggggaacagggtataaaggctcaatttagtcacatcatttccgtttctcacccaccccctttaaaccagatgtttgccaatgcattaacaatgcagatgtttcctgaaagaaagtttagtaactcaagcagacaccttattttcttttcaagcagaaaagactatgagatggtggttgtggttgttccgggagggagaagatataaatgatacacattatttcaaatcatttcatgacctcactgcacacttatagttattgtacctgttgtctttttgctgtcaagcctagctaagatcatttggaatgttcaagatcactcatacatgcatgtgcacacatacacatgcacatatgttcactccctatttcatccacatgaactaagattactgatgtgtacagattcaaagcacttttattcttttccaaaggcaagaagctgagctactttccagaatagttgtgaaagaccctgtcatacttctgcattgtttcctccacaccacctccatccagttccttatgaatggttactggttttcaaaaatatgagataaattgagtgtataaaagtcatttttagacaaaatgaaacaggaaatgaaagaaaccagaatctctcctcatttgtggatgggccagctccaccatgtcatggttaatctgcagggaggaaatactagatttgattgcagatcagactgcagcaaacctgctgtgactaaggcatcaagagaaagcaagcaacagctggggcttcagtggtgaaaacattatatatctagctttgaaatatgaaatactgtttagcagtgtcacctagaaaagagtgtttcaaaatgctgatgcttcataagaacctttctcttcagagttgtttcttttatctttcaaattagccagggtgggaaataaagtgatcacttggtgaagaaatctcacaaagaagaacatagagagttcactttcatctggagtaatgaacagattgaacaaactagaaatggttagtctgttaaagaaaaggtgtaggtgagctgtttgcaagagccacaagggaaaggggaagacaacttctttgtggacttaagggtgaaagttgcaagcaggcaagacgattctgacctccattaagaaagccctttccaaccaacaaccactgggttggttacgcaggttgggcagcattgggagcaaatgttgattgaacaaatgtttgtcggaattgttgacttaaagagctgttctgtcactggggacagcagcggctagatagccccattcagggagagggcatttgttcacctggccagagatcagagcaggctaagggactgctgggatcctgtccagctttgagaccctacagagccatgttcacctagcacgtatcccgtctgcggtcacgctcatttcttaccttattccagggctttcacctcagcttgccaggctggagccaagggccaacgcagccgcgccttgttcgcgatggtagcttcccaggagccccctatggttccggaacggcgctgccggccccatcctgtttgctacctcctaaagccaaaggcactggcgggccgggccagcttctaaagtcgcgcaaggttagaaggttccggacaggaacggcgtgaggccaatggaaggaggtacttcagtttccctccagatgcccagcgatgggctcagagctccttgagaactcgggaaaggaagcagggtctctgaagaaatacttcaggagtagaaagaggaagctagagggttaaatgcactacacaggaacagaaatgagtttttcttagagttagtatatgtctagaggtgtagtaaactaaaacaagtcttgaattgcataccgccacgtagggaagaaatgaaaacctttgaatattagtgaaaaaagggaaactgcaacgcctgtattactagatagctttcatcaacagctcaaaaccgacagatttaaagaagcaacaccgcattttggctttctaaagctttaatttggtttggatcccatgcccatgaccctgccagctgacaattctaagcatgcgcaaactggccccaaaaattcctcccacatttccgaagaactatttggccctttatgtgaagtacctggtttttccattttctgttttaccataggcctcagttcggtgtgtggcgtatttattctacatttaacaatttgaagatcattctattagattaaaaaaaaagaatacaatggaagccaagtgattaagctttccttatgcttatattaagttgtagcatatgcatttaccgatagttaaccgtattaacctacagaaaatgtccagggaaatggtctatttcttattctatttttgacctaaagaaaatctttaaaatgtcttagcattttccccagtctccatccactttcctcagctttggcctgaagctatctttaaaggtaccctgtacagctcttgccctgtacagctagctacagagattcaatcctttctgttcgattaggacacatctcagtggcagataacatgcaaagttattatatgtatgaaccagaacttgtttttccttaggggccaggatgttacactaaggtcttaagactatagtaatatcttcacttgaaaaagccctctattattcctatctcagatgataaaaattcaattaagagaaataagaacgtgacatgtgtaatcgcacctggctctacaaagctagtctggacagacatttaaacaattatcctctaagattatttgatgaaatgcatttcaatgactagttaaccattaaaaaccaaagtgagcatcccatctgttcccagtcaaatgacctagagcaaaggactaggcaaaccacatctgtgggcatagcaagctgtacatcacaaacaaatgaatttgctttgtatatgagtgagagcaaacactctttattgtacaacttgggtgggtaagtagggagaataatggttttactgaaatcgcaggtaacggttacgttggagttaaaggttaggaagaaaaccaaagggtaagagctgttgttctgggctggcattgtcaatgaagagcataaattcagatgtgaatgtatattttgtagaagcatgtgtgttgttggtttttgtgtatgtgtgagtctgaaagagggaaaacaggctcccattagactatgactaacaaaaatgtttgacagattataactcagatgtcttactcagagcatatgccttcccattttccccattattccccaacatgatgtctttaagaacttgtccttgaccgagcagacatctcataccccaaatagctaatattttgatagctatgatcctgaacggccaaacattccaaaaccaagtagtttgtaatatctttaaatgcaaatatattttaggccttttccttggcaaggatgtttggtcaggggttggcaaaaataatgctcttcagacttaaaagaacacaaccatatttcttagccatccaccagaaagtagtagaagctccaggaagcaagtctttgtcaggagtcagactagctacatcataatctctctgcccaggggctgtggatgtcatccatcctggcctaactagcctactgagctgagagatgtccaatttccccccaatacactaaccagaggagaaggaccgtgatatcattgcatgtgaattcttaattccaattgcttaaacaaatatgttcagttgtaactatcaataccagtatataacagtgttggccaagttttattgatgctgacaatcaattggagttacagccagacacatggtcttatgaccggcgtacttacgcagggctttgcactgagacaggtcgtgcatctgaggtttactgctttgcatttttgttttgtaactgaagtctgatgagacagccagagcatgtgctacctagggacttgaatccctgcagccccatttcacttctcaccaccttccggggtggtttctcgacctcccactcccctaccacctggtgccttagccagccctggctctccctccaaacacctgcccaatgagcactgccaccccatggtgcccagacatgctctccctcctcatccctacctagctaccattgccactcccctcccccagcggggacatgggcataggagcagggagagttaaggttggtcaggtgcacgtgccctatgctatcttggaagggggcttggccatgtggcatctctggaccaagaatgcgccacagcacatttggagggtgaatggtgggggcacaccccttgtccacctctatttcaggcatggaacacatcctggcatgaaagttgcagtcccttgggaatcacctctccaccttgattgccacagtaggccagtgacaagggaagattgacacatcatcccctgctggggcccagtgtcctgtggctggcaggcaggggatcctaaggacatgtgggtgttaaattgtagggtgcacttcctgggcacctttgagggtctgcactgccccagcaaatatccccatgctagaaggagcaaaatattaaatggcaaattttaaaaatgtaacaagatgggttgcaaaagagactacagaggaaagcaaaagttttgtattttagtatcttccatggcacttttcttcctagcttttgaacaaggggccccacatttttatttctcactgagccccacaaagtatgtagccattcctgcccggagtgaggacttttaaaacataaagattatcaagtcttggaaattctgattcagtagatatataacaggtctcaaacttaattatgtaaagaatattctggagagcttccttttacccagtcccacccaccaaatattctgataaattaagcttcgattagcccccagatctgcattttataaggatccccagatgattctactgcaattggtccacagaccatgcctggaccgaatttgggtgcttaggagcacaaattctggagccgggcagacttgagtttgcttcctagctttaccaactgatctcaggggagttaatgtttacctctaaactttagctcatgcatctataaataaatatattaatatcatgtcataaggatattatgttgtattaaatgtctttaaaacaccacaatgattagcccaaagtaaacactcaataaatgttcaaaaatttaggaaaattgttaagactgggttgtatgcacactggtgtttattatattatgtagttttttctgtatttttacaacatttcagaattaaaagcaacagctagaaaaagagggaaatggccgggtgcagtggctcacgcctgtaatcccagcactttgggaggccaaggcgggcggatcacgaggttgagagatcgagaccatcctggccaacatggtgaaaccccatctctactaaaaatacaaaaattaactgggcatggtggcatgcgcctgtagtcccaggagaattgcttgaacctgggaggcggaggttgcagtgagccaagatctcaccactgctctccagcctggtgacagggcaagactccgtcaaaaaaaaaaaagagagggagagccagagtatgaaaaaggaagtcagagccctttaatgagtcagctttgtaggtctccaggtaggaggctagtgcttcagtgtctaggacatagtaggtgttcagtaaattaaattcaggacaaaaagaacatgccccaaggaccatctgatatccacttaaagtgatggactacctcgtttcccttgtttatgaatgggttcatgcctaagactgtgtgcactttaatacaagggcagtcgttcagaactagtcaggtcctgaaaaggatttaccaaatgttgagtgtgccctctagtgttcacacttcccagctttcttcctataaaggtggatcaaggcacttgcttacaactggaactgaaatcctccaagtcgatctagacattgagatggagaaaatattcattgtcgactgtaattatgcaacgaatatccagttgagataatggacttgcctcttatctaataatacccaggctcaatgcgtcactgctttgtccactttgcccaaaattcaagcacagctaagttgatattttaggacaaaggcagcttactatccagccagaggggagtagaatatggttaagagagagtggaaagaatgaatgagccctgctattcctcactgcctggatggctataagcacagcccttatggaggccttaggtcttgcttcataatattccagtttgaaaagggtttgaaaagacctcctagaaaaatcagtagtttttctcttttgagtaacatgtagcaaaaaaaatttcatcatgtaggtacagggaacaccctaataactattaatctcaaggagtcaagccagtgtgtttcctaatgtatctgctgtatccccatgaagcaaattttgccatcagagaaactgactcatggggaaaaaatccaaggacctcaaatcaccaaaagaagccattcctcagatttgcctaagcttaagcttccctgtctctcattgtgtgttgctttcaatgcagttacataaatggcttttttgtttatgcaccaaaaacactaattcatctgcaaagctcacatttccagaaacattccatttctgccagcacctagaagccaatattttgcctattcctgtaaccagcacacatatttatttttttctagatcaaatgtattatgcagtaagagtcttaattttgttttcacaggttgttttgaatggtaaagttgatgcattctgtggaggctctatcgttaatgaaaaatggattgtaactgctgcccactgtgttgaaactggtgttaaaattacagttgtcgcaggtaaatacacagaaagaataataatctgcagcaccactagctctttaatatgattggtacaccatattttactaaggtctaataaaattgttgttgaataaattgggctaaaggcagaagggtcataatttcagaacccacgtcgcaccgtcctccaagcatccatagttcttttgatatacccctattatcactcatttcagtgaggtacaattagttcttgatgtagccatttccataccagaaggccttcccaaaaatcagtgtcatgtcaccgatccttttatctctggtgcttggcacaacctgtagcaggtcctcagaaaacaaacatttgaattaatggccaaatgagtttgtgctcaaaaaaggggtgaggatacttgaaatttggaaaatctaggataattcatgactagtggattcattatcaccaatgaaaggcttataacagcatgagtgaacagaaccatctctatgatagtcctgaatggctttttggtctgaaaaatatgcattggctctcattacatttaaccaaaattatcacaatataagaatgagatctttaacattgccaattaggtcagtggtcccaagtagtcacttagaaaatctgtgtatgtgaaatactgtttgtgacttaaaatgaaatttatttttaataggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggatttccaaggttaattcattggaattgaaaattaacagggcctctcactaactaatcactttcccatcttttgttagatttgaatatatacattctatgatcattgctttttctctttacaggggagaatttcatattttacctgagcaaattgattagaaaatggaaccactagagg

SEQ ID NO: 2 - DNA, 1272 bp, H. sapeins
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 3 - DNA, 2804 bp, H. sapeins
accactttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggatttccaaggttaattcattggaattgaaaattaacagggcctctcactaactaatcactttcccatcttttgttagatttgaatatatacattctatgatcattgctttttctctttacaggggagaatttcatattttacctgagcaaattgattagaaaatggaaccactagaggaatataatgtgttaggaaattacagtcatttctaagggcccagcccttgacaaaattgtgaagttaaattctccactctgtccatcagatactatggttctccactatggcaactaactcactcaattttccctccttagcagcattccatcttcccgatcttctttgcttctccaaccaaaacatcaatgtttattagttctgtatacagtacaggatctttggtctactctatcacaaggccagtaccacactcatgaagaaagaacacaggagtagctgagaggctaaaactcatcaaaaacactactccttttcctctaccctattcctcaatcttttaccttttccaaatcccaatccccaaatcagtttttctctttcttactccctctctcccttttaccctccatggtcgttaaaggagagatggggagcatcattctgttatacttctgtacacagttatacatgtctatcaaacccagacttgcttccatagtggagacttgcttttcagaacatagggatgaagtaaggtgcctgaaaagtttgggggaaaagtttctttcagagagttaagttattttatatatataatatatatataaaatatataatatacaatataaatatatagtgtgtgtgtgtatgcgtgtgtgtagacacacacgcatacacacatataatggaagcaataagccattctaagagcttgtatggttatggaggtctgactaggcatgatttcacgaaggcaagattggcatatcattgtaactaaaaaagctgacattgacccagacatattgtactctttctaaaaataataataataatgctaacagaaagaagagaaccgttcgtttgcaatctacagctagtagagactttgaggaagaattcaacagtgtgtcttcagcagtgttcagagccaagcaagaagttgaagttgcctagaccagaggacataagtatcatgtctcctttaactagcataccccgaagtggagaagggtgcagcaggctcaaaggcataagtcattccaatcagccaactaagttgtccttttctggtttcgtgttcaccatggaacattttgattatagttaatccttctatcttgaatcttctagagagttgctgaccaactgacgtatgtttccctttgtgaattaataaactggtgttctggttcat

SEQ ID NO: 4 - DNA, 20 bp, Artificial Sequence
  Synthetic oligonucleotide
acagtgggcagcagttacaa

SEQ ID NO: 5 - DNA, 20 bp, Artificial Sequence
  Synthetic oligonucleotide
gtatatattccatatttgcc

SEQ ID NO: 6 - DNA, 20 bp, Artificial Sequence
  Synthetic oligonucleotide
ttctgccatgatcatgttca

SEQ ID NO: 7 - DNA, 20 bp, Artificial Sequence
  Synthetic oligonucleotide
taaaaggcagatggtgatga

SEQ ID NO: 8 - DNA, 20 bp, Artificial Sequence
  Synthetic oligonucleotide
gtagatatcctaaaaggcag

SEQ ID NO: 9 - DNA, 20 bp, Artificial Sequence
  Synthetic oligonucleotide
tcagcactgagtagatatcc

SEQ ID NO: 10 - DNA, 20 bp, Artificial Sequence
  Synthetic oligonucleotide
ttcatgatcaagaaaaactg

SEQ ID NO: 11 - DNA, 20 bp, Artificial Sequence
  Synthetic oligonucleotide
cgattcagaattttgttggc

SEQ ID NO: 12 - DNA, 20 bp, Artificial Sequence
  Synthetic oligonucleotide
cctctttggccgattcagaa

SEQ ID NO: 13 - DNA, 20 bp, Artificial Sequence
  Synthetic oligonucleotide
aggttcccttgaacaaactc

SEQ ID NO: 14 - DNA, 20 bp, Artificial Sequence
  Synthetic oligonucleotide
atttaaacatggattggact

SEQ ID NO: 15 - DNA, 20 bp, Artificial Sequence
  Synthetic oligonucleotide
taggaattaatgtcatcctt

SEQ ID NO: 16 - DNA, 20 bp, Artificial Sequence
  Synthetic oligonucleotide
ggacaccaacattcatagga

SEQ ID NO: 17 - DNA, 20 bp, Artificial Sequence
  Synthetic oligonucleotide
taatgttacatgttacatct

SEQ ID NO: 18 - DNA, 20 bp, Artificial Sequence
  Synthetic oligonucleotide
gtacaggagcaaaccacctt

SEQ ID NO: 19 - DNA, 20 bp, Artificial Sequence
  Synthetic oligonucleotide
tctgcaagtcgatatccctc

SEQ ID NO: 20 - DNA, 20 bp, Artificial Sequence
  Synthetic oligonucleotide
tgattgggtgctttgagtga

SEQ ID NO: 21 - DNA, 20 bp, Artificial Sequence
  Synthetic oligonucleotide
agtcattaaatgattgggtg

SEQ ID NO: 22 - DNA, 20 bp, Artificial Sequence
  Synthetic oligonucleotide
acccgagtgaagtcattaaa

SEQ ID NO: 23 - DNA, 20 bp, Artificial Sequence
  Synthetic oligonucleotide
tgacctggtttggcatcttc

SEQ ID NO: 24 - DNA, 20 bp, Artificial Sequence
  Synthetic oligonucleotide
acctgccaagggaattgacc

SEQ ID NO: 25 - DNA, 20 bp, Artificial Sequence
  Synthetic oligonucleotide
gcatcaactttaccattcaa

SEQ ID NO: 26 - DNA, 20 bp, Artificial Sequence
  Synthetic oligonucleotide
tccacagaatgcatcaactt

SEQ ID NO: 27 - DNA, 20 bp, Artificial Sequence
  Synthetic oligonucleotide
cagtttcaacacagtgggca

SEQ ID NO: 28 - DNA, 20 bp, Artificial Sequence
  Synthetic oligonucleotide
atggttgtacttattaatag

SEQ ID NO: 29 - DNA, 20 bp, Artificial Sequence
  Synthetic oligonucleotide
tccagttccagaagggcaat

SEQ ID NO: 30 - DNA, 20 bp, Artificial Sequence
  Synthetic oligonucleotide
cgtgtattccttgtcagcaa

SEQ ID NO: 31 - DNA, 20 bp, Artificial Sequence
  Synthetic oligonucleotide
acatagccagatccaaattt

SEQ ID NO: 32 - DNA, 20 bp, Artificial Sequence
  Synthetic oligonucleotide
gaagactcttccccagccac

SEQ ID NO: 33 - DNA, 20 bp, Artificial Sequence
  Synthetic oligonucleotide
caagtggaactctaaggtac

SEQ ID NO: 34 - DNA, 20 bp, Artificial Sequence
  Synthetic oligonucleotide
gctcggtcaacaagtggaac

SEQ ID NO: 35 - DNA, 20 bp, Artificial Sequence
  Synthetic oligonucleotide
aagacatgtggctcggtcaa

SEQ ID NO: 36 - DNA, 20 bp, Artificial Sequence
  Synthetic oligonucleotide
atggtgaactttgtagatcg

SEQ ID NO: 37 - DNA, 20 bp, Artificial Sequence
  Synthetic oligonucleotide
ttccacttcagtaacatggg

SEQ ID NO: 38 - DNA, 20 bp, Artificial Sequence
  Synthetic oligonucleotide
aagaaactggtcccttccac

SEQ ID NO: 39 - DNA, 20 bp, Artificial Sequence
  Synthetic oligonucleotide
aattccagttaagaaactgg

SEQ ID NO: 40 - DNA, 20 bp, Artificial Sequence
  Synthetic oligonucleotide
ttgcacactcttcaccccag

SEQ ID NO: 41 - DNA, 20 bp, Artificial Sequence
  Synthetic oligonucleotide
atttgcctttcattgcacac

SEQ ID NO: 42 - DNA, 20 bp, Artificial Sequence
  Synthetic oligonucleotide
tgacataccgggataccttg

SEQ ID NO: 43 - DNA, 20 bp, Artificial Sequence
  Synthetic oligonucleotide
ggaaatccatctttcattaa

SEQ ID NO: 44 - DNA, 20 bp, Artificial Sequence
  Synthetic oligonucleotide
aattaaccttggaaatccat

SEQ ID NO: 45 - DNA, 20 bp, Artificial Sequence
  Synthetic oligonucleotide
tagaatgtatatattcaaat

SEQ ID NO: 46 - DNA, 20 bp, Artificial Sequence
  Synthetic oligonucleotide
ccattttctaatcaatttgc

SEQ ID NO: 47 - DNA, 20 bp, Artificial Sequence
  Synthetic oligonucleotide
cacattatattcctctagtg

SEQ ID NO: 48 - DNA, 20 bp, Artificial Sequence
  Synthetic oligonucleotide
gaagatcgggaagatggaat

SEQ ID NO: 49 - DNA, 20 bp, Artificial Sequence
  Synthetic oligonucleotide
gagaagcaaagaagatcggg

SEQ ID NO: 50 - DNA, 20 bp, Artificial Sequence
  Synthetic oligonucleotide
aaacattgatgttttggttg

SEQ ID NO: 51 - DNA, 20 bp, Artificial Sequence
  Synthetic oligonucleotide
tgatagagtagaccaaagat

SEQ ID NO: 52 - DNA, 20 bp, Artificial Sequence
  Synthetic oligonucleotide
ttgatagacatgtataactg

SEQ ID NO: 53 - DNA, 20 bp, Artificial Sequence
  Synthetic oligonucleotide
aagcaagtctgggtttgata

SEQ ID NO: 54 - DNA, 20 bp, Artificial Sequence
  Synthetic oligonucleotide
tgttctgaaaagcaagtctc

SEQ ID NO: 55 - DNA, 20 bp, Artificial Sequence
  Synthetic oligonucleotide
gcttccattatatgtgtgta

SEQ ID NO: 56 - DNA, 20 bp, Artificial Sequence
  Synthetic oligonucleotide
ttgaattcttcctcaaagtc

SEQ ID NO: 57 - DNA, 20 bp, Artificial Sequence
  Synthetic oligonucleotide
gctgattggaatgacttatg

SEQ ID NO: 58 - DNA, 20 bp, Artificial Sequence
  Synthetic oligonucleotide
actataatcaaaatgttcca

SEQ ID NO: 59 - DNA, 20 bp, Artificial Sequence
  Synthetic oligonucleotide
acaccagtttattaattcac

SEQ ID NO: 60 - DNA, 20 bp, Artificial Sequence
  Synthetic oligonucleotide
atgaaccagaacaccagttt

SEQ ID NO: 61 - DNA, 20 bp, Artificial Sequence
  Synthetic oligonucleotide
catactgcttccaaaattca

SEQ ID NO: 62 - DNA, 20 bp, Artificial Sequence
  Synthetic oligonucleotide
atgttcacctgcgacaactg

SEQ ID NO: 63 - DNA, 20 bp, Artificial Sequence
  Synthetic oligonucleotide
cacagaacatgttgttatag

SEQ ID NO: 64 - DNA, 20 bp, Artificial Sequence
  Synthetic oligonucleotide
aattgcttaccaacatactg

SEQ ID NO: 65 - DNA, 20 bp, Artificial Sequence
  Synthetic oligonucleotide
ggcctggtgattctgccatg

SEQ ID NO: 66 - DNA, 20 bp, Artificial Sequence
  Synthetic oligonucleotide
atggtgatgaggcctggtga

SEQ ID NO: 67 - DNA, 20 bp, Artificial Sequence
  Synthetic oligonucleotide
tgtacattcagcactgagta

SEQ ID NO: 68 - DNA, 20 bp, Artificial Sequence
  Synthetic oligonucleotide
tgaattatacctctttggcc

SEQ ID NO: 69 - DNA, 20 bp, Artificial Sequence
  Synthetic oligonucleotide
acattcataggaattaatgt

SEQ ID NO: 70 - DNA, 20 bp, Artificial Sequence
  Synthetic oligonucleotide
gccattcttaatgttacatg

SEQ ID NO: 71 - DNA, 20 bp, Artificial Sequence
  Synthetic oligonucleotide
aaaccaccttgttatcagca

SEQ ID NO: 72 - DNA, 20 bp, Artificial Sequence
  Synthetic oligonucleotide
atcaggaaaaacagtctcag

SEQ ID NO: 73 - DNA, 20 bp, Artificial Sequence
  Synthetic oligonucleotide
aatggtttcagcttcagtag

SEQ ID NO: 74 - DNA, 20 bp, Artificial Sequence
  Synthetic oligonucleotide
tccaccaacaacccgagtga

SEQ ID NO: 75 - DNA, 20 bp, Artificial Sequence
  Synthetic oligonucleotide
aatcacatttcgcttttgct

SEQ ID NO: 76 - DNA, 20 bp, Artificial Sequence
  Synthetic oligonucleotide
tgaggaataattcgaatcac

SEQ ID NO: 77 - DNA, 20 bp, Artificial Sequence
  Synthetic oligonucleotide
gcaatgcaaataggtgtaac

SEQ ID NO: 78 - DNA, 20 bp, Artificial Sequence
  Synthetic oligonucleotide
aaatttgaggaagatgttcg

SEQ ID NO: 79 - DNA, 20 bp, Artificial Sequence
  Synthetic oligonucleotide
ttgtagatcgaagacatgtg

SEQ ID NO: 80 - DNA, 20 bp, Artificial Sequence
  Synthetic oligonucleotide
gttgttatagatggtgaact

SEQ ID NO: 81 - DNA, 20 bp, Artificial Sequence
  Synthetic oligonucleotide
tggaagccagcacagaacat

SEQ ID NO: 82 - DNA, 20 bp, Artificial Sequence
  Synthetic oligonucleotide
tccttcatggaagccagcac

SEQ ID NO: 83 - DNA, 20 bp, Artificial Sequence
  Synthetic oligonucleotide
cccagctaataattccagtt

SEQ ID NO: 84 - DNA, 20 bp, Artificial Sequence
  Synthetic oligonucleotide
ggataccttggtatatattc

SEQ ID NO: 85 - DNA, 20 bp, Artificial Sequence
  Synthetic oligonucleotide
ttaatccagttgacataccg

SEQ ID NO: 86 - DNA, 20 bp, Artificial Sequence
  Synthetic oligonucleotide
ctttcattaagtgagctttg

SEQ ID NO: 87 - DNA, 20 bp, Artificial Sequence
  Synthetic oligonucleotide
acaattttgtcaagggctgg

SEQ ID NO: 88 - DNA, 20 bp, Artificial Sequence
  Synthetic oligonucleotide
tggagaaccatagtatctga

SEQ ID NO: 89 - DNA, 20 bp, Artificial Sequence
  Synthetic oligonucleotide
gaactaataaacattgatgt

SEQ ID NO: 90 - DNA, 20 bp, Artificial Sequence
  Synthetic oligonucleotide
cttcatgagtgtggtactgg

SEQ ID NO: 91 - DNA, 20 bp, Artificial Sequence
  Synthetic oligonucleotide
agtataacagaatgatgctc

SEQ ID NO: 92 - DNA, 20 bp, Artificial Sequence
  Synthetic oligonucleotide
ccatacaagctcttagaatg

SEQ ID NO: 93 - DNA, 20 bp, Artificial Sequence
  Synthetic oligonucleotide
aacttagttggctgattgga

SEQ ID NO: 94 - DNA, 20 bp, Artificial Sequence
  Synthetic oligonucleotide
gtaagctataccatttaggt

SEQ ID NO: 95 - DNA, 20 bp, Artificial Sequence
  Synthetic oligonucleotide
aagaaagttctcaaatgagc

SEQ ID NO: 96 - DNA, 20 bp, Artificial Sequence
  Synthetic oligonucleotide
agttacttacctaattcaca

SEQ ID NO: 97 - DNA, 20 bp, Artificial Sequence
  Synthetic oligonucleotide
catgttacatctaaaagaag

SEQ ID NO: 98 - DNA, 20 bp, Artificial Sequence
  Synthetic oligonucleotide
agttacaatccatttttcat

SEQ ID NO: 99 - DNA, 20 bp, Artificial Sequence
  Synthetic oligonucleotide
gctagcagattgtgaaagtg

SEQ ID NO: 100 - DNA, 20 bp, Artificial Sequence
  Synthetic oligonucleotide
acgcgctgcataacctttgc

SEQ ID NO: 101 - DNA, 20 bp, Artificial Sequence
  Synthetic oligonucleotide
atgttcacgcgctgcataac

SEQ ID NO: 102 - DNA, 20 bp, Artificial Sequence
  Synthetic oligonucleotide
aactgccgccatttaaacat

SEQ ID NO: 103 - DNA, 20 bp, Artificial Sequence
  Synthetic oligonucleotide
taatgtcatccttgcaactg

SEQ ID NO: 104 - DNA, 20 bp, Artificial Sequence
  Synthetic oligonucleotide
ccaaagggacaccaacattc

SEQ ID NO: 105 - DNA, 20 bp, Artificial Sequence
  Synthetic oligonucleotide
cactatttttacaaaactgc

SEQ ID NO: 106 - DNA, 20 bp, Artificial Sequence
  Synthetic oligonucleotide
aatccatttttcattaacga

SEQ ID NO: 107 - DNA, 20 bp, Artificial Sequence
  Synthetic oligonucleotide
aattttaacaccagtttcaa

SEQ ID NO: 108 - DNA, 20 bp, Artificial Sequence
  Synthetic oligonucleotide
cttattaatagctgcattgt

SEQ ID NO: 109 - DNA, 20 bp, Artificial Sequence
  Synthetic oligonucleotide
cccagccacttacatagcca

SEQ ID NO: 110 - DNA, 20 bp, Artificial Sequence
  Synthetic oligonucleotide
aattccaatgaattaacctt

SEQ ID NO: 111 - DNA, 20 bp, Artificial Sequence
  Synthetic oligonucleotide
gttaattttcaattccaatg

SEQ ID NO: 112 - DNA, 20 bp, Artificial Sequence
  Synthetic oligonucleotide
caaaagatgggaaagtgatt

SEQ ID NO: 113 - DNA, 20 bp, Artificial Sequence
  Synthetic oligonucleotide
aatgatcatagaatgtatat

SEQ ID NO: 114 - DNA, 20 bp, Artificial Sequence
  Synthetic oligonucleotide
tcaggtaaaatatgaaattc

SEQ ID NO: 115 - DNA, 20 bp, Artificial Sequence
  Synthetic oligonucleotide
agaatttaacttcacaattt

SEQ ID NO: 116 - DNA, 20 bp, Artificial Sequence
  Synthetic oligonucleotide
aaccatagtatctgatggac

SEQ ID NO: 117 - DNA, 20 bp, Artificial Sequence
  Synthetic oligonucleotide
gtgtggtactggccttgtga

SEQ ID NO: 118 - DNA, 20 bp, Artificial Sequence
  Synthetic oligonucleotide
caggcaccttacttcatccc

SEQ ID NO: 119 - DNA, 20 bp, Artificial Sequence
  Synthetic oligonucleotide
agaatggcttattgcttcca

SEQ ID NO: 120 - DNA, 20 bp, Artificial Sequence
  Synthetic oligonucleotide
agttacaatgatatgccaat

SEQ ID NO: 121 - DNA, 20 bp, Artificial Sequence
  Synthetic oligonucleotide
caatatgtctgggtcaatgt

SEQ ID NO: 122 - DNA, 20 bp, Artificial Sequence
  Synthetic oligonucleotide
gagtacaatatgtctgggtc

SEQ ID NO: 123 - DNA, 20 bp, Artificial Sequence
  Synthetic oligonucleotide
tagattgcaaacgaacggtt

SEQ ID NO: 124 - DNA, 20 bp, Artificial Sequence
  Synthetic oligonucleotide
tggaatgacttatgcctttg

SEQ ID NO: 125 - DNA, 20 bp, Artificial Sequence
  Synthetic oligonucleotide
tcagttggtcagcaactctc

SEQ ID NO: 126 - DNA, 20 bp, Artificial Sequence
  Synthetic oligonucleotide
ccagtttattaattcacaaa

SEQ ID NO: 127 - DNA, 20 bp, Artificial Sequence
  Synthetic oligonucleotide
ttaaggacatgttcctgctg

SEQ ID NO: 128 - DNA, 20 bp, Artificial Sequence
  Synthetic oligonucleotide
ctaaattgagcctttatacc

SEQ ID NO: 129 - DNA, 20 bp, Artificial Sequence
  Synthetic oligonucleotide
attaaagtgcacacagtctt

SEQ ID NO: 130 - DNA, 20 bp, Artificial Sequence
  Synthetic oligonucleotide
ggtgctggcagaaatggaat

SEQ ID NO: 131 - DNA, 20 bp, Artificial Sequence
  Synthetic oligonucleotide
gtgtatttacctgcgacaac

SEQ ID NO: 132 - DNA, 20 bp, Artificial Sequence
  Synthetic oligonucleotide
tcaacatactgcttccaaaa

SEQ ID NO: 133 - DNA, 20 bp, Artificial Sequence
  Synthetic oligonucleotide
tgttacatctaattcacagt

SEQ ID NO: 134 - DNA, 20 bp, Artificial Sequence
  Synthetic oligonucleotide
caaaacaacctgccaaggga

SEQ ID NO: 135 - DNA, 20 bp, Artificial Sequence
  Synthetic oligonucleotide
ctatggaagcaagtctgggt

SEQ ID NO: 136 - DNA, 32767 bp, Mus musculus
tcccaattacagataaggaaactgagttttttttcaggttaaattaatggtgaataattttaaaaatatgacaagtcacaggctgatacatagttactattctaaacattatatataaatatatataagaaattaacataatagaccaaataaaaggattcacttgttatctttaaactctatattattgctctgtgatgaagataaactgagttcccacagataaacttacctggcagcacaattgtagctctgctccagtggcagcagctcatgaaaccagagtgcactttttgttttgttttgttttgttttgttttgttttgttttgttttgttttgttttttgtttgtttgtttgttttgttttgttgttgttgttgttttgtttgtttgtttttcaagacagggtttatctgtaaagccctggctgtcctggaactcactttgtagaccaggctggcctcgaactcagaaatctgcctgcctctgcctcccgagtgctgggattaaaggcgtgcgccaccaccggccggccacttttttttattattagatattttctttatttacatttcaaatgttatcccctttcctggtttcccctccaaaacccccctatctctttccccctctccctgctctccagcccacccaccccttcttcctggtcctggcattcccctacactggggcatttgagccctcacagaaccaagaacctcagagtgtactttaataacaagccctgaccattctcttcacttgatccaagaaccaactggaaatagcccaaagatacaccgagggagatggacaacaatttcccagaagtaagtcccattcagcttgcactttggaacgattgattagccctgacgcttgcacaatctcctaacaaaggtcatgaagcacctgaacaccgtcatggcagaatccccggctctcatcaccatcttccttttaggatatctactcagtaccgaatgtgcaggtttgtttctttttcgatgctatgcttaatatgcttctcctttaaaaacagaaatatttagaactacctttatcaagtaaagcaagatttcatgatttggcagctatagtttctattgtagcaggaagcacgtcagccaggaactctggctctgtggtaacttcagtttcaatatttgaaaaaacgaactacatttacagggcttgtggtgcatttttatttaatagatacagttattcttatgatttgacaatataattccacttaaaatttatgtgtccttggttttaaaaaaatgaaaatcggaaaatgtcttaacaaagaaattgcacaataagcaggaaacatgttattttaggattttagactcatcaccttgaaataaaaaggggaaacatcaaaaccacaatggttaatttgcataaaaagtctttcttgaatctacatgtgttccagtgttttagaactagatggtaaggcatagttataatgtttaaacatatatttatagtttaatttctcagctaaaattgaaagattacgcttttattactaagttgctgtctgtaatgaaagcttttacatagatggaaataaatcagctcatctgtcatacatctcagagctgatgtttagagaaccataatactcagaggcctccctaggggacaaaagtaagtatacaaatggccagaaagaggagtagcttaattcatgcaaagtacagcacatcagggtttcgtttggtttcgttttttgcaacaacagtggcattactccccagggaaaggcacacaggaagaacttactggctttagacaaacagcctgattgccccagatgcatttatcacacttactagtcaacctactaaagtcaagcaagtgacagtgccaggatcagaggtgccaaccctgagcatccccagaaaatcggctggtctcctggttcccactccagacatgatgtcaactgtggaatcagacttaaaagctgaaatgatgatgtgagaatgcaaaggttaaactgcactagcaaagtgggtagagatgaaaaatacatgttggaaactcattacgtggaccataaaggcaggtcattcaacctagtgcatcagattgacctatgtgagaacaaaagatccaagcactctcttataaagatccaagcacgtcttagtggtgagggaatctcagatgtaaaatgatagaaaaaaatcatgatttcagagctgtggcaataatgactaaagaggtgttaaatatatgatccccagacagtattgaggataaaaagaacaagattgtgagtcagtttggtcagggaaccagggaaatggccctggctggggagtggggctaggtattcataaagcacactcttctgttaaacggtcctgggctttagaacagagaggccccatgttcataactactgccttattgtgacaaattagaattgggaaatcaaaattagtctgtgaaatgaatttgtgcttacaatgtatgcaaatcaaaattatctttaatgcatgtatttggatattagtccatactgttgtggttcgtggtactgtgacaaggtactgtggcagagatgaatgggttcagaaaaaaagactttaatgggaagggatgaggtgtgtaccacatgcttaggaagtacaaaagaaggaacataatggatgtaaaatctcctgaaagatacagtgtttggagcaagatctcaagaaatgaatagttgcccactatccttaaatgtctgacctggatggttcattgggcaaaactgctttccaccaagcctaacaacctgagtttgatcccgagggtctatacagtgaataagagacttgacttccaggattgtcatttaatccccacatgtatgagaacacttacaatgcatgtatgcataaatacagatggatgtatatacatgtatgtgtatatgtaaacacacacacatattcttagggtttatgtagtcattttaaacaccatgaccataagtaacttggggcgaaaaggtttatttcagctgggggaagtcagagagggaactgaaaccagacaagaacctggaggcaggagctgatatagaagccatggagaaatactgcttactgtcttcctcctcaaggcttgctccgactgctttcttacacaactcagaaacaggatcaccagcttatgagtagcacatccaaaagtgatcttggccctcccacatcaatcatcattcaagaatatactccacagtttttctcacaggccagtttggtgatggcatatttcaaattgaggttcctcttccaaaatgactctagcttgtgtcaaattgacataaaagctagccagaacacacacacacacatacacacacacatacacacacacacacacacacacacacacacacacacacacacacacacacacacacacactgagagaacaataaaaatagtcactaatgtggagcattttgtgtagatagtaaccctcacagagaaagatctgtgtatatataataatggaagtattcaagatgcttcatctggtttaaatattgactgttagaagaagtaaggcaagaaaatggggtgagccaaatcatccaaaaagggttctaagctaagagagatggaagagtagagagcaatgtggaggtaaattgtaagaaacatatgtgtaagaagcacaccctcactgtgctataacactcagtctagaacactattaggaactgatgaggctccaggccagggagtccaaccttaagtctcttgcaatcatccagtggaaacaaaacagcataatgtcgacaatagtaataaagagagtactgacaattggagtgatgattataaaatagtgctgatatgatttaataggcacatgcatatgaaaggtttgggagacatatatggagtgtctggggataatgttataatatcaacacagcacagggtctgtagtctgacttctgactgtaattacaggctgatcctttattgtgtgacaatcagtcgggaaatacacgaagtcaaagaggttctttccatggttactaccaatgatgacaatggtaattttcttgcacagagagcagtgagaatgtaaagacacgttgaagtgcatagaagcactggagaagggagaaagctaagaagggggagggggagatagcatgagcaacttgggaagaaagtgagtaaccaggatacacaagcctataattttctgagatcatcaacatgatgatgaggtacatgaccttacacttctgttacattcattttacgttttaaaaagttaaggtccagccagacattgtatggtttgtccatgttcatgcagtcagtaagagttagatacatgttagatacatatatctacatactgagggagagagacaggaaaaggaagaaggcagaaaactgaaagatttcaatcctcaaaaaacctcatcatgaatttcagtgaaatgcttccatcactggcattcatatgccaactaaagacatcatgggcctgaagataacatcaattatgccatgattattcctcacgatcttatgagaagcattggagtcatatttttagctggcctacagttgggaggttgttgttgttgtttaatatattttcatacaaaatagtttgatcatattctttcccctcccccaactcccagatccttcttatctctctacccacccaacttcatgctctctttttctccctccctctctccctcccttcctccgtccctcccttcctccctccacctcctcccgctctccctctctctccaagcaaaaacaatgaaaatcaaaacaaacacaaacaggcttctgtccttcgcgggatatttgttgcaaagtcattgggagactacacactggaaacagcccagccagaggaatgatagatcaccttggaacgatcctgtactgagctattttcaagagatgacaaagtgggaacttaactgcttagttgagaactttctttttcatccaaagttaaacacaaatacaactgaaatgtaaccttttattgctggcactctaccaattaaaaataaaattgaattttaattcctaaatttccatgtgtatactgtgaaaaaaaaatcatagtttttggctctatgccccaaagagaaattagctatggaattacatgaacccaaaacaaaccttttctttaaaaacatatttagttttctgaggtttttttttttcctaatactaaagaactatacttttaaatttcagttttccttgatcgtgaaaatgccaccaaaattcttacccgtccaaagagatataattcaggaaaactagaagagtttgttcgaggaaaccttgaaagagagtgtatagaagaaagatgtagttttgaagaagcacgagaagtttttgaaaacactgaaaaaactgtgagtatacccacatcatacctgaataatatgtcccagagcaaaagatagaaaatctctattttcaaaaggagtgtggaactagagaggtgactccatatttaggagcactctgctattccagagcacacagattcaaattcaagcatctaggaaacccaacactctcttctgacctttttgagtagtacatacacagggtgcatgtatgcacacagatagaacactcatacacataaaacaaaatacaaagtattatagatcaattattaaaggaaaattgtatttcaaatcttaaaaatatcagttcatatcatatcatctattacaaatgttctaaaaggaaaggaatccatatcaaaatactttaaacactatcattaagctgtcctcctttttccttacagactgaattttggaagcagtatgttggtaagcaattcattttattttattttatttcctacctgctatatgaaacacttgagaattgtgccttttttctatatagagaggttgtacagtctcagtaaaaaaaaaaaaaatcaggaaaaaaaccaaacaactgcatcttagagctaaatgtacatttactgtaactagtagattcagagatgattgggatggtttccaatgccctccgtgtcttgacctaccatcccttcttgttgccctctgaccacctctactactactgccctcattccacaaaggggctccttgcatgccctagcactgttcccatattagaggaacaatcttactatccctgttagctgagtcttcccagcttcttctaagctaacttccttaccttgctcagggacttgtccaaaattcctccaactcagtaagtcctcccttgataaggtattaatattccaacctgctacctggtcgctctgtcattttagacacatgtgttctggtctattatatgcatatatggaatgaaatatatttggtatgagacccaattctaaatgcataatttgttatattcatatatctatttataagaatatctaaatacattttaaaacacgttgcagaaagatcctgtcatacaataatctttatagttttgtttatgaaacaatttcacagcatgatattttcctcttgcagcaccatgttggtacccaaacagttttagattttggagtgatttgaaatttgagctttcagattgaggatactcaacctgtagcactttccacaacagtgtcttgttcgttttcccaccctcctagagtgcaagcttcatttcagcaggagtcagtagcttcacttgttgataggctcctgacataacactctctacaatggaggcactcaggaaatatctgcccaattaactgacccaaaggaagaaatgcaatactctaacaataaccaattttattattttctattatatctttgtacactttcaagtattgtccacaaatacaaatttcaaaaaaattgaagtccagaagttggattttgagggacatttcacaggctgacttctttcagagtatcttcatggacagaacagataaacttggacattattcactgagctccatctacctgaggtccaattcaattttctagaattccatttctggagaaactactagaaaatttcataccctcataactcttatttctcctctttccatttaaagttgtgaaagaaaagaggttctctttggccttccttatttttaaagatgacggatactaagcctacttctgagttagctgacaaatttcatgcaaactatttatactcaagttaaatagtggaagcttaatgcttgtctttaaatgcaaaccattgggagcttggtcctcatactccaatcattaattatgaaaaaaaaaatttacaggccaattgatggaggcatcttctcaattgaggttctttctttcatggtgacattggcttagttaacaaacaacgaaagctgttcaaccaggacagatgaaaaaccttactttgttcctcataacccaccattttttgttgtttgcatatatttggtatatacagtttcagggatgatcattttgtattagagaaccaactaaaaggctgatacttggagaggtttctctctctctctctctctctctctctctctctctctctctctcttcctgccttctcccttccttccttccttccttccttccttccttccttccttccttccttccttccttccttccttccttcctgcctcccttcccacctctcttaccagttgcctgtaattgttttgtctaggggtgagactctgagacttttttccccttccacattaacatgtccattgatattgtcactgttcaaatcttgtttatacagctatttccagcagagactgttttacggcagacttcctggtgctccaactcataatctttctgctctctcttctgtgatgctcactgagccatacatgcaagaactgtaatacaaatgtatttaatagaactaggtttctctgatcattgatctctccattgtgtccagttatggtttgctattgtgatctccatttgctgtccagagaagcttctttgatgacgggtggtagctacacttagctgtgggcataaggagaagatttagaatgtagttagggactctgctgtctagcaaagtgatagtagtaggttctcttctaagagccatgacctcagtaaccctaaaaccatatggacaaaaagaacaaaaatgacccagcaggttgtagttaaatatttttgcatttatttttatgtaacaataataatcaaagaaaatgaattatcagtttgagtgtgggaaacatggaagagtttgaggaactggtttgagagatacaagggaaagggggaaagtgtttgaattgtattttaattaaaaggcagtgaaaataataaaaataaaataaatgctaaaaaaaaataacagctaggtgtgggtggtacttgcctataatctctgcactgagaaggtagtgagagaaaaatctctgaggcttgctgcctagccagcttagcctaatcaatattctccaagttccatgagagaaaaacaatgtaatgactttctctccagttccaaaaacaatataataagtgaaaaaacataccaagggttgacttctacatacacacacacacacacacacacacacacacacacacacacacactgagagagggagagagggagacagagaaggagagagagagagacgagagagagagacagagagagagagaacgagagaagaaatcagtgctacagttggactctcatgatcccttcacactgcccatcctgtacatgccccagtcctgccccagccctggccatggccccagccccggcttcggcccgagccccagccacgaccacggccccagtccaagcagcatactttgaagttggacaatggctttccaggtcagctaagtagagtgtttactataccttggttacaaatacccatgagacatcaaggacatgcttctgttattactattttcactacaaacacctaaaacaacacagacaagaacaaaagagcaataatgcccatacttgttttcacattgtagttaagattcttacctatcacaaaattagcctgtactaatcagaaattgcctgggccaggctcctcactgcaacgtctggcagtctctaccatgacctcatcatacaaagaagccaactgaactaccatctatcaggcattttaaggcatttttattagatattttcttcatttacatttcaaatgctatccccaaagccccctataccctccccctgacctgctccccaacacacccactcccgcttcctggccctggcattgttgaatacagttcacagaagcagtttataaaatgcctatcccattcgcctgtcacagcagtcccacaggtaaagcattatggaaggaaaccgagaaggaaacaggcttggagggggcaagggatttgccttggatcaccaaactgggaagtcacaagaccaggaaaggaacccaagttatctcctaaccaactcttctttcatataacatcttatgatcatgaaatctgaatgtttttgcttgattgttgttgttgttgttgttgttgttgttgttgttcttcttcttcttcttcttcttcttcttcttcttcttcttcttcttcataagggtttatttattttcattttatgtatataagcatgccatctgaataagtgtctgggtaccccatatgtgcctggtaactggaaacaagaaaaggggtatcagattccctgaaactgtagttgtggactgcaataaacaaccatgttagtgctgggaattgaactcaggtcctctgtaagagcagcaagtactcttaaccactgaatcatctctccatctctagaacccagtaatatttaaaagagaatctcacatggcagggtcttgtctcctgttgtctttgacccctactaactatggctttcttctggcctatagtacacagagacaagaaagcaaaggaacactaaattcaaaagaaaatgacactcattttgaagatatgtctcagcaaatgagtttcctgtatggctggctgcaagcccagaccctttcactaaaacaccctaaataattcagattcattggtctataatataaagtgcaaatgtagctcatttttagaccagttctgagcatcaataataataaaccagagataatgtattttgttttcatacaattggaacaaattgcagtatctgtgcaaagcacattggatcaaggggcagagagggcaaggtctaatttttacagcaatccaattttccagagagtttatttctgaaaaaggatactagctccatagtcgacagaccatggacacgtgtttccatcagagaacagggaatgtggtcgtctacacaagaaatgtcaaggggtttggctagaaagtatgactaataactagaaatttctctttggttcttctcgtttttctctccttcataccatttcatctcattgttttctttctcatatgtggagtcttacagacatagctcaaaatacatagccctctccttctggttgggttcatcctatcccttgatgctttcttagtagtatgagcggaggccagacactaagaaaccagacttaaaggtttccttctgttcctgacccttctccatttgcctattcattctccctgtttttgtttgttctactgtcagcattttcaaagtctctgtaaatgtagaaactttggaaaacacagaatggaatggatacagcaagtgtattcctgaaggtcacccaatagtactttaagcttctgatgatacacataacatagttgcctacttggaacctaactagtatgcaaaaaacctattatctttatgcttctggcaagtttgctgtcacagccccttatttcatatgatacataaacattaaaaggcaatacaaggaagtagatacatttgccctatatcagtgctaccagtccatgagtgaaaacacctttatcttctaacatggtttgttgtacaagaccttcaataaaatgtcctcgtatgaaatataaatagtattcattgaagtggtttaattcaatttaatttatggttaacttgtccccattccatgaagaaacactgatattccttctcattgtatgctgaatataatatttccatacaaacacatttccccctctttccttggattatataaacagtgtatgcctccttctgtgaaatttttgaggctggcattagcctgaaaacctttgctgcagtgacttttagaaacttggaaaagcaggcacatcacttgcctatatgcagatagcttgcagctcagtagttccctaatcctaaaatcagaatcctaccctcttaaatctcttcaggggaggactgggcattttaggcagctctctgataattcaatttcttaacctgtcttaaagatggagatcagtgtgaatcaaatccttgtttaaatggtggaatatgcaaggatgatattagttcctatgaatgctggtgccaagttggatttgaaggaaggaactgtgaattaggtaagtaaccttttatgtattcatattcaaactttcctttttaaaccagatgaaacaggatatttaaatgtctttataccacacaattctcttaaaacacattttctatgcttataaacatttactttctctatagtcactttctaggacaagaattcaagctagtatttaggggtaatgttccattcactttatgtattaatccctaataatccttaatatgtcttcagtaccgttattatccctcactttatgagcaagaaaactttagctcaaatatcaaatataaaaatggttgagccaggaagtgaacttcttgtactgtactcaaaactctgagaaaactgcacccataaaaagattggagcttgtgactgagttactgtggatttagataaaggaagtgaatagcctatagagctgggcatggcaggtccccatgtaataagaactcttccagatgaacaaaacattttataagttaaatttgttgtaaaaatcagaggttgttccttgattcaatgtattgattatatcgaagagtaaacagaagaaaaaagtatcttgcaaaggcaagatttttgtatactttcctggaatttttttttttttgagattttggtcatttgagcagtaaaaccccaaggtcctttatctccttagttactgcctctggatgcagtattgatgatacggtggttgagaagaagcacatgggaccaagaaaagatgacacactccgaaattctgggagcataaatgtacaatctgagggaaagactctcacaccgcatggctaaggatagaaaggagagaggtagcaggacactattaaggcaatatagccattgctctgacacacgaaatgaaaggcggcagcagctggactgctcctattgagaaaggggatgaaagtccctgtaatattgttcaataccaaggggaagtgcattgatcatctttctttgagatctgaaagatctcagaataaaaaggaaagttgaaaggacaaaggaattgaaaaagaaagaatgaacagaagtgggtgagctaacatactcagactggacttgcatttgtttaggattcctggtgtggcaataaaccactgcttgagttcccactccttgggggtgggggtgttaattggttcaacatccctaaagtgccattctttctgaaagattataatttcaaattccatataagtgaaactcagtcctcatattccagatacatacagaagttcttcgagtagttactctaaaaccattggagtaaaggggatgaataacagtctttgctgtaagtgaaggttacagtgtagtaggtggtaattacaaacataaaggtagctcagtggttaaggagcactgactgctcttccagaggacctgggttcaactcctagaaccaacatggtggctcacaactctctgttccaggagatccaaagctctcttttggcatccacaggcaccaaacatacacatggtgcacgaatttacatgcaagtaaaacatactcctatatgtaaaaatataataaaatatattttaatttataaacttttaaaaacataagtctctaattcattttttacactccatattccattccccatctctccccatccacactctgactgctccacattcctcacctcctccctaccccaccccatctccacatggatgcccccatcccccacctcacctgacctctaaactccctggggtctccagtctcttgaggattaggtgcatcatctctgaatgaacacagacccagcagtcctctgctgtatgtgtgttgggggcctcatatcagctggtgtatgctgtctgtttggtggtccagcatttgaaagatctcggggtccagattaattgagactgctggtccttctacagaatcacccttctcctcagcttctttcagtcttccctaattcaacaacaggggtcatctgcttttgtccattgcttgggtgcagataactgcatctgactctttcagctgcttgttggatcttttcgggggcagacatgctaggaccctttttgtgagcacttcatagcctcagtactagtgtcaggccttgggacctccccttgagctggatcccactttgggcctgttgctggaccttcttcaggttcctcaccatttcaatccctgtaattctttcagacagaaacaattatgggtcagagttttgactgtgggatggcaactccatccctcacttgatgtcctatattcctgctggaggtgggctctataaattccctctccctactgccaagcatttcatcaaaggtctctccctatgagtcctgggagtctttcacctcccaggtctctggtgcattctggggggtccacctaacctcctatttcctgaagatgcctgtttccattctttctgctggccctcaggacttcagttcttttacctcacccaatgccagatcaggttctcctctaaccccccactgcccccctacccggtccactttccctcccaggttcctccctcccttcccactttcttttctctcccaagggggactgaggtgtcctcacttggtcacttcagcttgttgagccttttgaattctgtgaactgtatcttgtgtattctgtatcttttttttccttttggctaatatccacttattagtgagtacataccatgtagccacaaatagtataaaatatcttggggtaactctaaccaaacaagtgaaagacctgtatgacaataacttcaagtctctcaagaaagaaatcgaagaagatctcagaaaatagagagatctcccatgctcatgggttggcagaattaacatactaaaaatggccatcctaccaaaggcaatctatagatttaatgcaatccccatcaaaatcccaacacaattcttcaaaaacatagaaagagcaattctcaaattcatctggaaagtcaaaaaaacccagaatagccaaaacaactcttaacattaaaagaacagctggaggaatcaccatccctaacctgactcaagctttactacagagcaatagttttatttcatatttcaggatattttgtataaatggttatctgtgtgccatgtgcatgcaatgcccatagaggccagaagatggcataggatcctctgcaactgaagtaaaaggtggctgtgagcctccatgtggcctttggaaactgaacttggttcatctgaaaaagtagcaaatgctaccaatgagtcagttctccagccctctatcttattttaaaagatagcttaaaaggaggaaaagaatacagttgaggaatgtggacataagtttggagttctataacaggatgcactgtggttggggaggttatggtccacaaagaaaacttgctaagtcagcaactggcaataactcaatgccaattgaactccttggttcccacatttctgtttggtcagggctctagctatactgcagccaccaccatgcttagcctctagggtattttgatcagatccaaccaatttgagaaagggttttttcaaaaaatcttttcagctcagctgattcagaaacaaaatgggaattctgtcagaataccctttatctctgatactacaggagagcaacaacaaagcatagacaaactcccatttaagcttcttgcctccagctgaactaaacacaaatctgaaaagtcacaaggcatcacaggtatgtgggcccatgccttaccttgtcttgagaaaggaaagatttccactattgtgaccctttgctctttttcccctgtccatcaggaaggctcttagttgggggagtaaggagcaaatatttggggcatgctttctagttagaaaacaattaactggctactgggtccctttggctaaaataaatatctttgaatttcctctgctgcagaagagatgcaaatgtccatcaggcagattaaggtctgcaagaatctagtcagcctttcctcccgaagaatgcagaaaaacaaagtggaaagtaaaaattacaaacaaaagaatagtttctgggggtcttttgttgtttgcttgcttgctttttcaatttttcagcacaccacagccccaactgtctcatttattatgtattttggggttgggaaaaatgttgaggttagccgaataagatgttttctggcaaacctgttcaacaaaattgtgttaaggttccggtgagcaaccctctgctatcaggaagagaaagaaaacacatgctttaaatggttcctcagtggatattctaggtcccaaggccacccttgggactaagaaaaacccaacctcaacctagcgtgtggcatctaagtgctggaaaattgactgagttcagttctgtttctcaggtcctgctctattgacctctcccctggaatttcttgtgctatgattttatttctagaatcctgaaccttttgactgcaggcaaaagcctgctgatgttacagcaatacatccatcccatcacacgcatcaaacaagccaaagatcaagagtagcagcaacatgctgggccagaaagggcaatagcaaacaggaaaaaggttgagaggtctggttacttaaccaaggacacagaagcagaatagaaaaatcacaccaagtttactcattccctagaacacaactagcctgttctccccacccacctctgttaacttatcacaacctcatattgagggaacattttctacaggtagtatttgatttggctgaacactttagtattgctttgtagcaacaatgtgatagccaggaacaaaaaaaagtaaccagagatgttattaccacttgaggaaaagacctcaaagctaaggaaatacaaaagaccatatgccatcttttttgtcataaatttagtggattaatggtagcaaaaatgaaatctaaggtcagttcctttattcccttcccctgataatagtttattttattttcctcccctgatttactggactatgcaattaaaataagtaaacatacagaaaagattttgtatatgaaggcaatagaagtagcaattatatttgtaccaaaataaacagaatataaattttggaacagtgtaaaaattgaatgcttagtaataaaacatatacaagtactctactcatactctggcttctttggatatatacacacacgcacatcagtattttattttattttattttttcaaaaacattgcccatttctttttctttttttaattagatattttcttcatttacatttcaaatactatcccgaaaatcccctataccctcccctgccctgctccccaacccacccactcccgcttcctggccccagcattcccctgtactggggcatatgatcttcgcaacaccagtatttttaacaaatagtagaataggtgggtgaaaatgtattaatacaatgggactactacacagcaatgtaaatgaactattggtttacaaaaccacatagatgaatctcataaataaattgctaagccataataatatatgtgctgtttatataaagctcatgaactatcaatagtgtttacaatgtttgaaatgcaggtaatgactcttgtaatggagggaaggtagaatggctgggagggaacattatggaacttggggcaattgatgatgtcctacttactaacctatgaagcattaaaacaggtgcacttactttgtaaaacttcacacaaaggttttattgacttttcctggatttatgtttcactttaataaataggtctctaatcatagttcatgatttggttaaagtacaatttccaaatatttagttgtacacaatctcaaaatattgctgacaccatcgtcacacctttctgagaatcttattttacttcatctttcttctcttagttggaggctcccagattgtttcaacttgtgttgaacccatacatgagccagtggttccgtatacttttttttgaagtgtatgtcagatgagccattacggttttttttttttccttttcttttcttttagatgcaacgtgtaacattaaaaatggcaggtgcaagcagttttgtaaaaacagtcctgataacaaggtaatttgttcctgcactgagggataccaacttgcagaagaccagaagtcctgtgaaccaacaggtcaaaatctaaataagttctttaaagaaaacttgcatctaaatcttaagcatttaagctacactattttaggctttttaaaaaggatttatttattttcattttatgagtgtcttgtctgcatgtttttggtttgtttgtttgtttgtttgtttttgttggggtttttttttttggctattaccagtatttttttacttttagtttaactatgaacaaaattgggttgggctaaattaacaactttagtttacatgagcatttacttataagtcattttgatactaagtacattgaataaaaaagaaaccacaattacatatagacagataatatgcatacctattttttatgccatgcatgtgctatgcttctggaggtaaaaaaaagatgatatcagatcccgtcaaactggagtctcagaagtttaatgaggtactgtgtaggtgctcactaacccaccataaccccagctataacccgggttcagatcctctgcccttagattaccttattccagtagtgctggcatcaactcccttttgtaactgaacaaaagattagtatcttgaaataaacaaattaactttccaaatgtcaccataatttcacactttaataattatgtacagcttgaattcttttctagggccatgtttttttttttcctctgatttctgtattacaaagaaatcgatttctgtgatcgctatatgcaatatttgaggttcagtacatcaaatcaataatacaaaagacatataaatacttggttgactgataggtttccaattaaaaaaaatgatgctaaatgttaagcataagtctggtggagcctaagtacttccgtctatgaaaatgacttcctgacttgtgtcatcaactgaatttttgaacttgctgtcacactagattcagccactgttacatgctttcctaggctgatctacatcgtctttacagtaaagggaaaggcaaggcttcatagctgtgggagcataccaccaggtcgacacctaattcctctatttagaacatctccacactcgtttcttacctctgaaaccacagttttgattcagagaaagggagagaagctctgtaagactagcagaaagagaataaatcagttaatatgccagcctgaggagccgaggtcagatactcagtatagaaaggccaggcagtgtgagcccagcactgactgaaaggagagggtaagataggaccagctagaaatcatgagctccagactcagtgagagatccagtctcaaaagggtaatttggagagcagagaaataagtgaagatgtttgatgttaacccctggcttccacacatccaagcatggacaatcataccagcacatacatgtgcccacacccatatagcatacttacagataaaatataaacacacttttaaagcaagccagatagagcttctcttatgtttccaaaaacagatcttttattaaattgtgaaccactcccctgtaactgtgggtttgatacctatctcagcaatgtaccaggcagaggaaattaaagaaaaggacagaatacaacagtgcacctgctacaaacaaaaccaagaatctctgacgtttcaccttctgcctgcttcttcacatgtactgacttacccggcccttgccttagaagtgtgtctgtgtttgtctctctgtgtgttgtgtatgtgcacacacatgcacacttaacatttaaaaagctaacttctcatgaaggcagctagtgcagaagtttagtccttgtaaacacttttatgttctaagagaggaaaaaaaattgaaaagtgtctccaactattaagagttctatttgaatgtagccttagctttagcagagtaactaggccaaacctaaaatagctgttctgccttttgaaagatgaaggcccctcttttcaagccattgttgatcgtgtgtatttgtgcataagtattttgaactaatttcctatttttttcaaccacaagctgctttcatgatacttagccacggctggttgctatagaaatgcctggtacatgaaatgtatctaagaggagagtaaaacatgaaaatgaagtgtgaggtgactttggctacaaatttccattttggtggtccccggtgtaccagtagataatttgttgagaaataagtcaaaaattccatgttggaatctctagtgcataaataatctacaaaaaggccctattaataggaagtagtgagagcctttagcttctcctaaaaggaaaattccacagcataccttcttgatctccatccagggcaagggaattttgaaggaagactttaattaagaaactagatagaaagtaattagatttctttttttaaaaaatctaataaatttgattccagaacatctattttatttccatgaagatggtggatttcagagcccaatgtttcccatggaagctttgaaaactttacatgaaaatactacagtcagccttttagtttgaaaaaaaaaaaaatatatatatatatatataaaacatcgacagactcaatgtgatagcaatgaatgaggctcattctcaattattggaatgcatttctatatgccacggtggaatatcagaatattctcttttttctatttttgtagttccatttccatgtgggagagcttctatttcatacagttctaaaaagatcacgagagctgagactgttttctctaatatggactatgaaaattctactgaagctgtattcattcaagatgacatcactgatggtgccattcttaataacgtcactgaaagtagtgaatcacttaatgacttcactcgagttgttggtggagaaaacgcaaaaccgggtcaaatcccttggcaggtactttatattgatcatgtgtctaactaaaggctggggctcagatgacagaagacaggccagctgaggaacagggctaggatattagaaagacctgtgggcattggagaaaaattaggtaaattgcatcactcggcaaaataaaaagccttactggtaagcatatggtgaaagacaagtccagcagcagctaccagacaatgttccaaagaatttggcattaaacaaatagcacagtagaattccaaacaacttattagcaataggcatgtccataaatgaggtctctctgtctctctctctgtgtctctgtctgtctgtctctctgtctctgtctctgtctctgtctctgtctctctctctctctctctctcacacacacacacacacacacacacacacacacacacacaaacacacacacacacacacacattgggactcctgcataccctcaggccctaggaaaaccaatgagatgatctctaggcacagactgactaactgatggggataatgaaaagggacatatgtggaaaggattaaagcaaaacttacttcttagaataagcacatgggctctgatctactgccacatttctccaagtctgacagtatgcccagatcactaggaatttgtcaggtacacagtctcacactccacctcaatctccctggatttgaacaactagagagtgaggccctgcaatctgggttcaaggaagccctgcagatcattctgatactgtagtttagaaacccactgtcctaaaggacacacacagaaacctgtctccaaaatatcaaataatggagtcaatctatgctgtcctccaagtgtgttcaccacagaaactgcatatacagtattacctgtgcatattgttttaaactaagagttagattaggagcaaaacccaagccttaactatatatttaaccaaagcccaagtgattccatcagagaggagagacaggccacctcctacctcaatgaaaggagccagtccagaccttacataaaattgggggtcagtacatgtggatactttacttaactagttaaaaacattaaaattttttcagtttctttgtcacttttgccacacttccagggaataatggccacatgtagttggtcactattttactggacaaggcaggtattaagagaaattttccatcattacatgtatttctgttagatagtacatatcatggtttactctagcttaaaactccttgggtcataattataatttagtagtgatttcaatgattcttcacacgacaataatttctctaataaaatatgaacttctgatagcttaaatgacactgcaatcatcaaattagctacaatttaaggaggtcaggtagatgtttgcatctaaatatcacatttaacagcactagaaatttctgaaactagctatgtatactagctatgggtctatcaagaattcttcgaaatgtctgctactctgccattttataatatactagatgacagatgtcaaacatgtatttggggatagaggaaagtcatatagacactaaacatgtaggcaataaaatacactgtgtatacatttatataccctctggccctccttaagggcaccataccagagctacttcccttatcccgcaccatcctttcctttgctctcacagctttattgttcttcactgttcttagtcaaactaatcctcttcagtgacctttctttaaacaatctctcttgggtaatggcacagtaatgttttcattctatttgacatcagtatagttaactctatttttttgttttgttttacattctcgtgtatttttgttccttatatccctaatatacatgtataaggtggtttagattagttacctttctgttgctgtgacaaagcaccataaccaaaggcgacttaaggaagggagaacttactttggcttgtgattccaaagggacagagtctatcacagtggggaggcagggcagcaggaactggaagctgagagatcacatcttgaaccacaaacatgagacagagccagaaaacaggaagtagaatgaggtgatggattctcaaagcacacccgccagcaaggtgccccagctccccaaaaagcgccaatacctgggaaccaagtggtcaaatgccctagcctatggggaagacatctttcatttcaaccaccacagtgtccctaaccccgccccccaaatcctataactttgatcctccatgtattttaggggcataaagcaatttgtccagagacttattgactaacaacatcatttcctctttaaataaggtagaatgcttttaatcccaagatgttgtgttaacattgcccctcattaaacaaaatagaaaaaaacacagttaaaccagaactaaaatgggggacatagtctcagttttgtcacataatctgtatttttccatccactctccttagagcagatgcttagcaaatacctatagaaataatttcttaaagaaattttagtaaatctagaagagactactaggtggtggttgcactatttcaaatgttaccccctctcctggtttcttagacaaagaagaaacaggaagtgaaagcaaccacggcctcttctcacttgggaaatgatcagctctgccacattgggtaatctgtcacaagagagtactatagtttttaattagcttatttattctacatgtataaatattttgcctgaacatatgtctgtgtactacatctgtgcctggctcccagagacatccggttcccctagagctggagatgcaggttgttgtgagccaagatggagatgctagaaacaaaacatgtccttgataagagtgccagagccatctctccaggataactagatttaattacagacaagactgcacccagatgttgtggccaaggcattgtgagaaagtgagagctgacgtcttcaatgatctccacactacatctctggttctgaaatatgaactaccatttgcaactgtcaccaagaaaagtgtctcgagttgctgatgttgcccaaaggcctttccattcggacttgattcttttgttggggaagtcagttggagtaagggacaaagtgagcactcaggagtaaagaaatcccacaaagaacaaagaattttctttcatcaggaattaccaacagtttgaatgaactagacatgtttagtctgttaaagggttttggtggtgctggtggtatttcagtagtagagtgctttcggagcatcagtgtaaccttgagttaaagtctcagcatgcacacatacacacacaaacacacacacacacacacacacacacacacacacaaatttaaatgatctgtccacatcttgccaagggggtggggaatagaaaaatatctgtttctttgtcagcttacaggtaaaagttgtaggtaggtagtctggtctcaattaagaacccttttttgagtccctgactcatttgctaggctggtcagcttttacaggagccttgtttgagcaaatgttttggcattgtcaactggcagagctcttctgacccctggagtagcaggtctagcctgagagtcagagaatgttcatccatccatgagaaacccatggacacaggttagaggatttccagagcctggctgtcactctcttttctcacaccattccagggcttccacttcttttgcctggctagtcgccagggactaagtggcctgtctaagcagactgtcaggatcactttctaggaactctaaggcttctgagctgcactgctacccagttcaggctcaaactcctcccctcaacccctcaacccctcaacccaggccaaaggtgcaagccaggcagcagaggtgtttctttcaggttctcccacagtgagatcggagcttcttggaaaagaagcagggattgggaaggagtactacagggttggatgggaagccagagagtcaagtataccacacattaacagaaacatgtttttcttagaaactagctgactgagaaagctaaaccagctatataggaaggactaaaagctttagaacattagagggagggaaacgcaaactgtaccgcctatattactggattgttttcctcaacaactccaaagtagaggtttgaagaaacaacatagcgcctgtcttaagacttcttatagtttttcttccatagtcacagccaatctacagctagtttgaacatgtgtaaacctttttttttattattcctgctaatatctgtggagactatacaaatacctgatctttttcaaagaggtagttttatcatttttgtagtcttggcacaaatatccttaaacaccttccttttccctattccctactttcctcagcttggtccccaagccatcgttgaggtgacctgagctctttgcttatgctgcatctgagagatctactccacaatagacagtagccttcaagtatgaatgagccagagatgttttcttttctatgacagtcataattctacaaaaatgttttacttatccatgtgtggtggtgtgcacatcttttatgttaccattcaggagaaaaagcaggtggatctctgtgagttcaaggccagccaggtctacagagttccaggataaatgggctacatagtgagaccacgtctcaaaagcaaaacaaaaaaaaaaaatgtcttccttaaaagagacaagcttatgttattcctagcatagacgataagatcccaattagaaaaaaatggcaagtgtaatctcatctacaaagccagtttgaacaaaacatttggactattaacttgcaggattgcttgatgaagtatatatcaatgtctagtttatttgttggttatagttaaccattagaaagcaaagcgagtattccttctgtgctcaacaaaataacctactgccatgaatttggcaagcaaagtttgtgggtctatcaaatagtataccatgcaccttagtttgtataacagaaagtatagaatgaatggtgttgttagatagctgaaatagatggcgatggagaacagaggttttagggagctcacaggaaaggctaggctagacttaaagtaaaaagcaagaacaatgagacaaaagccattgtcatgggctgccattgccatgaagagcagaaatacagattgtatgtattttctcagtgtatagaggtgtgtctgaggtaagggtgttagacagaagcaagctcctactagactgtctccatgaaaatatttgacagatcagaacttggatgtcttgcccagaacatatgctttcctattttccccatcattctctgaagtcttataagaatttgtccttgtccgggcagatggctcatcttccaagtagctaatatttaaatagctatgatcctgagatggccaagcaaactttccaaaaccacgcatttgtaatattttaattgcagatatatcttagtccttgtttcttggcaaaggcgtttgctagaaaaaaattctaacatcaattaaggattggcaaagagtgctcttcaggcttaaaaggacacagtcatctactggaaagcagtagcatctccaagcaacaaacctttggcagagtcacatagaatacattaccgtctatgtgcccaagggctatggataccattcaggttgacctaactaacacactaagctgggagctgttcagtattgccataacagtacccaaaaaagaaatgaatattctactgcaacatgttaattttacttccagagacaggtgtgttggattataggattgtgaaccatgtagtattagaatcatgttacccaaattcacttgataaaaattatttggcatcaggatgaggcacctcatcttttgaccagtacacttgtacagtgctgtatattgagaaggagaaggataccacatcaaatattaagtattttgtacttatattctgtaatggaagtctaatgagacaatagaacatgtgctgaagacatgaaaccttagattattgtagtcacatcttttgccacttttgactccactacccatgatactttgtaacaactacctcttcccacacacttgcccagtgactactgccatctccctatccacgttactccggaaaattccctctcatcactccaacacaaatgccattgctgctctcccttcctccagggttggcacaggcagaggaaagattgaggtcaagtacccacactcgtatcatttggtggaatgtgcataatcacatagcatctctgaaccaagagaattgttccttctgagcatgcagaaagtgaagactccgggtcccatacatgcttccaaagaagtgcatttgttttagaaacacatttctgccttggtggctgtggtattccaccaaggaaagagtgatgaaccctcccctgttaggaatggtgcaaggtatatagagaggtgtgtctgaagctagtagtgtggtatcaagactatgtgtgctgagctgcagactacagcccctgggcacatgtgaatgaaggcttgcagtcagccaacaaacatccccatgcaggaaggagtatagcatttaaattagtttagaatgatgggacttgaaagctgccctagaaaaaaggggaatattttctctttcagtatcgtcaataccacttatatcctggttttagaacaacgggcctcatgtattattatttcccattgaacctataaattatgttttcatccagagtgaacatttttaaaacataaagattacccagccatgagaatttggattcactagccatgcaacacttctcaaacataattgtgagaactataaaaatatttaggagtttcattttttttcttcccacaaaatattctcatcaagtctctacgaacctgcaaatctgtttttataagaaactcagaccattctaccacagttgtttctcagacaatctctaagaaattctgaatgttcaggagcataaatactggagccaggcagactgtctgtgtgtgtgtgtgtgtgtgtgtgtatccatatgccacaacacacatgtggaggttagaggccaaattgcaagaatcagttctctcttgcttctatatgagtcccagggattaaactaacgtcatcaggcttggtagtaagtactttaactctctctgccatctcagtagcatgagtgggtatgtctctttaaaccttaattttcaaaaaaaacaaaaaacaaaaaacttaatttgctcatctgtggaaggataaagtaagattctatgcatgttttgtgttttgagttccaaatgtgtataagtaatacaattattagcaccaaatcatctaggttgttcattatataattaggtttttctgtatttttgccacttttgaaatataaaccaatcgctataaagagcgagagagattaagtgtgggaaagaaggcaagagacctgtaacaatttggtgagtcccttaggattctatttacaaagctagtgtttcagtgtccagaacacagtagctgatcagtcagttgaattcaggcccagagcaaccttcaaaaggaccatttgtgccccacactcaaagggaccaacagccctctttgtcttgcttaggaacaggttgtttataacttaggcaaaagggcatcggctcagcggctggcgggctactgctcagtttactgaatgctgagtgtgccctctagtgtccacaattttcagctttttttttctggtttttttttttcttacttttgtttattagatagttccttcatttccattttaaatgctatcccaaaagtcccctatactctctcccctctctgctaccctacccacccattcccacttcttggccctggcattcccctgtactggggcatataaagtttgcaataccaaggggcctctcttcccaatgatggccgactagtccatcttctgctacatatgcagctagacacatgagctctgggggtactggttagttcatattgttgttccacctatagggttgcagaccccttcagctcttggatactttctctagcttctccattggggaccctgtattccatccaatagatgactgtgagcatccacttctgtatttgccaggcactggcaaagcctcacaggagacagctatatcagggtcccttcagcaaaatctttgtggcatatgcaatagtgtctgcgtttggtggctgattatgggatgggtggggcagtctctggatggtccatcctttcatcttagctccaatcaagaaggaaaaccaatgtgtggatacttcatttctccttagaatatggaataaaatatctatggaaggagttgcagacacaattttcagctttatctctgaaaatctattcacaagatcttcatttatatcaaatctggaattctcttaggttgatttagtaaaaatgttgatgatcctctttgattatgatgtaaatatagacaagaaagaataaattatcctcttaactataccgtccatgtttaatgtcacactttaaacactcctgtccacactgtgtaaaaatcaagtgcagcaaagcagcctttttagagcaagaatagcttacacttccagccagaggttaggggagatggaaggaatgtgtggatcctactgatgatcagctcataggtgactaataaacacaggtcctgtggagtccttaggtcttagaatatatttcataatattctaattttgatagtgcttgaaacagctcctgaaaaacaaactggtcatttctctctttcaagtgacaagtagcaaagagatgtcgtcatacagggccagagacttcatagtaattattcatccttggtagttagtcagtatttcctctcatatacactgtagctccctcaataacttgtgtcagcagactctgtagatagcatgggaaaggaactcaagggccccacatcacacaccagaaaacagcactcctcagattcacctcacctggagaaccatccccaccataccacctctcattctgcattcacttcaagatcattacagaaacaacagtccagtttgtgctctaggaatactgattcatctgcagatctgacactgtccagagatactttatttctgcccagcacctagaagtcaacttttccacacccctgcaagtgtaatatagatccatttttctctggatcaaatatcatatgccgtgatcttcttaactttatttccacaggtcattttaaatggtgaaattgaggcattctgtggaggtgccatcattaatgaaaaatggattgtaactgctgcccactgtcttaaacctggtgataaaattgaggttgttgctggtaagtaaacaaaatagataatccttagcaacattagtgcatgatggacatatcacatgtacaaatgtccaacggtgttgttactgagtaaactgggcaaatgggagaggggccataattccaaaccataacttatttcaccttccaagccccataattcttttttgtgaacccataaagcactctcagcatagtaaaatgatttattgattaccaaattccccacccactatattagatcattgagatttagttgctaatgatcagtgaagccaaccagactggggaccatgggaaatgcatttatgtgaaggactataaactatgagatttgttttcaacaggtgaatataacattgataagaaggaagacacagaacaaaggagaaatgtgattcgaactatccctcatcaccagtacaatgcaactattaataagtatagtcatgacattgccttgctggaactggataaacctttaatactaaacagctatgtaacacctatctgtgttgccaatagggaatatacaaatatcttcctcaagtttggttctggctatgtcagtggctggggaaaagtcttcaacaaagggagacaggcttccattcttcagtaccttagagttccactggtggatagagccacatgccttaggtccacaacattcactatctataacaacatgttctgtgcaggctaccgtgaaggaggcaaagattcgtgtgaaggagatagtgggggaccccatgttactgaagtagaagggacaagtttcttaactggcattattagctggggtgaagaatgtgcaatgaaaggcaaatatggaatatatactaaggtttcccggtacgtcaactggattaaggaaaaaacaaagctaacttaatgaaaaacctatttccaaagacaattcagtggaattgaaaatgggtgatgccctttacagactagtctttctaccttttgttaaatttaaatatataagttctacaaacactgatttttctctgtgcataagacaagcccatctaggatctatattgttctagagtaagtaggttagcaaatataatcactagagaaatagtttagtaagagattcaccatttctgtaagtccagcccttgttaaaattagaaagtaaagctttccgtgttgcccataaggcgtgatggttcttgatacagagatgtacccaattctccctccttggcagcaattcatgttttagctcttccttgctactctcaattttattagttttctatccagaatctttaacccatttatggccagaagaatacaagagcagctgaaaaattaaaactcatcaaaagcatgacttcctctcctgatttttctgaatcttgtatcttttacaactcccaaaccacaaatcactgacctctccgtcattctcaccttccctttctccatcaccactgaaggaggaagctatatgagttccaggacagcctaggtacacagagaaacccggtcttgaaagaaaagagagagtggggagagagagagagagagagagagagagagagaggagaaagaaatgattaatttaatcatattggtaatatatatatattatatctctaaaaaaaagtcactaaaccttacttgtaacaactgcctatttctatggtgtaaatatccttactttggtagatttcaagctattaacatgaagttactggaaaaggagttgagaaaacatatggaaaattactcttaaaactgtttcaggcagtttttaacctagaagcagctgaactttctaggaatacttcaacagtgcatcttcagccttctccagttccaacctacctaagggtcatgtctctcacagcaggctcaaggctgcaagagtcattgcaaatggccaactgacttgcccatttatggttttcttctcaccggtaaactgttattgtaattaacactgtcatattgaattttctagagggatgctgaccatccgacccatttctcatctgagacttggtgaactggcattttaatacttatctggacctttgtagtgatgcataattggtttgaaccccttgtcactgccacctgcccccaccaacacaaaatcctacttcattactgctgactctgctaacgttccactacttgttgcctcttttgtcttgcaagaagtatcaataaacatctttccagatttccccaagtgtttcttgttaaatcatttagagcggatcccaaagaacacaatcaacaaagctctgaaaagaatgtgtccgatgagatgtaactctgactttatgctctatgtcgcaaaatttcatgcaaggagcaatgattcaaatccacagataactcacatccccgttgaggaattaatacctccatttgtataatgagagattttgaggaaacactattaggaggcctttgagatgttaagaatatataggttttttttttttgttcttcatcccttcattcaacaaatattcaaagagaggtctcctgtgtagtgtcacaaaaaaaaaaatgctgattacacgttgaaacaagtatatttgtatttggaaaatttacaaaaatatgtgtaaattaccatcagtgcctactacattatgacagtaattgcatgataaatgttagctgggactacaacgatgactcagcagttaaaagcacttgcttcttgtaaatggcccaggttcaattaccagctccacatcaagagacttaacaaccatttgtaactccacctccaggagat

SEQ ID NO: 137 - DNA, 20 bp, Artificial Sequence
  Synthetic oligonucleotide
ggagaattgggtacatctct

SEQ ID NO: 138 - DNA, 21 bp, Artificial Sequence
  Primer
tcctgcactgagggataccaa

SEQ ID NO: 139 - DNA, 21 bp, Artificial Sequence
  Primer
tcccacatggaaatggaactg

SEQ ID NO: 140 - DNA, 29 bp, Artificial Sequence
  Probe
ttgcagaagaccagaagtcctgtgaacca

SEQ ID NO: 141 - DNA, 20 bp, Artificial Sequence
  Synthetic Oligonucleotide
ccaatttacctgaattatac

SEQ ID NO: 142 - DNA, 20 bp, Artificial Sequence
  Synthetic Oligonucleotide
gaacaaactcttccaattta

SEQ ID NO: 143 - DNA, 20 bp, Artificial Sequence
  Synthetic Oligonucleotide
ggattggactcacactgatc

SEQ ID NO: 144 - DNA, 20 bp, Artificial Sequence
  Synthetic Oligonucleotide
gcatctgccattcttaatgt

SEQ ID NO: 145 - DNA, 20 bp, Artificial Sequence
  Synthetic Oligonucleotide
gcttcagtagaatttacata

SEQ ID NO: 146 - DNA, 20 bp, Artificial Sequence
  Synthetic Oligonucleotide
tgttatccaaaatggtttca

SEQ ID NO: 147 - DNA, 20 bp, Artificial Sequence
  Synthetic Oligonucleotide
ctttgagtgatgttatccaa

SEQ ID NO: 148 - DNA, 20 bp, Artificial Sequence
  Synthetic Oligonucleotide
gggcaatgtcatggttgtac

SEQ ID NO: 149 - DNA, 20 bp, Artificial Sequence
  Synthetic Oligonucleotide
gttttggttggagaagcaaa

SEQ ID NO: 150 - DNA, 20 bp, Artificial Sequence
  Synthetic Oligonucleotide
atgagttttagcctctcagc

SEQ ID NO: 151 - DNA, 20 bp, Artificial Sequence
  Synthetic Oligonucleotide
ttcatccctatgttctgaaa

SEQ ID NO: 152 - DNA, 20 bp, Artificial Sequence
  Synthetic Oligonucleotide
agaaaaggacaacttagttg

SEQ ID NO: 153 - DNA, 20 bp, Artificial Sequence
  Synthetic Oligonucleotide
tagaaggattaactataatc

(19) United States
(12) Patent Application Publication
(10) Pub. No.: US 2011/0059895 A1
(43) Pub. Date: Mar. 10, 2011

(54) MODULATION OF FACTOR 9 EXPRESSION

(75) Inventors: Susan M. Freier, San Diego, CA (US); Brett P. Monia, Encinitas, CA (US); Hong Zhang, Carlsbad, CA (US); Chenguang Zhao, San Diego, CA (US)

(73) Assignee: Isis Pharmaceuticals, Inc., Carlsbad, CA (US)

(21) Appl. No.: 12/742,076

(22) PCT Filed: Nov. 5, 2008
(86) PCT No.: PCT/US08/82527

§ 371 Date: Sep. 16, 2010

Related U.S. Application Data
(60) Provisional application No. 60/986,781, filed on Nov. 9, 2007.

(51) Int. Cl. A61K 38/16; C07H 21/00; C07H 21/04; A61K 31/7088; A61P 7/02; A61P 9/10; A61P 9/00

(52) U.S. Cl. 514/14.9; 536/23.1; 514/44 R

(57) ABSTRACT

Disclosed herein are antisense compounds and methods for decreasing Factor 9 and increasing clotting time in an individual in need thereof. Examples of disease conditions that can be ameliorated with the administration of antisense compounds targeted to Factor 9 include thrombosis, embolism, thromoboembolism such as deep vein thrombosis, pulmonary embolism, myocardial infarction, and stroke.

### SEQUENCE LISTING

The present application is being filed along with a Sequence Listing in electronic format. The Sequence Listing is provided as a file entitled BIOL0100WOSEQ.txt created Nov. 5, 2008, which is 112 Kb in size. The information in the electronic format of the sequence listing is incorporated herein by reference in its entirety.

### FIELD OF THE INVENTION

The present invention provides methods and compositions for lowing levels of Factor 9 (F9) in an animal. Such methods and compositions are useful as anticoagulants.

### BACKGROUND OF THE INVENTION

The circulatory system requires mechanisms that prevent blood loss, as well as those that counteract inappropriate intravascular obstructions. Generally, coagulation comprises a cascade of reactions culminating in the conversion of soluble fibrinogen to an insoluble fibrin gel. The steps of the cascade involve the conversion of an inactive zymogen to an activated enzyme. The active enzyme then catalyzes the next step in the cascade.

### Coagulation Cascade

The coagulation cascade may be initiated through two branches, the tissue factor pathway (also “extrinsic pathway”), which is the primary pathway, and the contact activation pathway (also “intrinsic pathway”).

The tissue factor pathway is initiated by the cell surface receptor tissue factor (TF, also referred to as factor III), which is expressed constitutively by extravascular cells (pericytes, cardiomyocytes, smooth muscle cells, and keratinocytes) and expressed by vascular monocytes and endothelial cells upon induction by inflammatory cytokines or endotoxin. (Drake et al., Am J Pathol 1989, 134:1087-1097). TF is the high affinity cellular receptor for coagulation factor VIIa, a serine protease. In the absence of TF, VIIa has very low catalytic activity, and binding to TF is necessary to render VIIa functional through an allosteric mechanism. (Drake et al., Am J Pathol 1989, 134:1087-1097). The TF-VIIa complex activates factor X to Xa. Xa in turn associates with its co-factor factor Va into a prothrombinase complex which in turn activates prothrombin, (also known as factor II or factor 2) to thrombin (also known as factor IIa, or factor 2a). Thrombin activates platelets, converts fibrinogen to fibrin and promotes fibrin cross-linking by activating factor XIII, thus forming a stable plug at sites where TF is exposed on extravascular cells. In addition, thrombin reinforces the coagulation cascade response by activating factors V and VIII.

The contact activation pathway is triggered by activation of factor XII to XIIa. Factor XIIa converts XI to XIa, and XIa converts IX to IXa. IXa associates with its cofactor VIIIa to convert X to Xa. The two pathways converge at this point as factor Xa associates factor Va to activate prothrombin (factor II) to thrombin (factor IIa).

### Inhibition of Coagulation.

At least three mechanisms keep the coagulation cascade in check, namely the action of activated protein C, antithrombin, and tissue factor pathway inhibitor. Activated protein C is a serine protease that degrades cofactors Va and VIIIa. Protein C is activated by thrombin with thrombomodulin, and requires coenzyme Protein S to function. Antithrombin is a serine protease inhibitor (serpin) that inhibits serine proteases: thrombin, Xa, XIIa, XIa and IXa. Tissue factor pathway inhibitor inhibits the action of Xa and the TF-VIIa complex. (Schwartz A L et al., Trends Cardiovasc Med. 1997; 7:234-239.)

### Disease

Thrombosis is the pathological development of blood clots, and an embolism occurs when a blood clot migrates to another part of the body and interferes with organ function. Thromboembolism may cause conditions such as deep vein thrombosis, pulmonary embolism, myocardial infarction, and stroke. Significantly, thromboembolism is a major cause of morbidity affecting over 2 million Americans every year. (Adcock et al. American Journal of Clinical Pathology. 1997;108:434-49). While most cases of thrombosis are due to acquired extrinsic problems, for example, surgery, cancer, immobility, some cases are due to a genetic predisposition, for example, antiphospholipid syndrome and the autosomal dominant condition, Factor V Leiden. (Bertina R M et al. Nature 1994; 369:64-67.)

### Treatment.

The most commonly used anticoagulants, warfarin, heparin, and low molecular weight heparin (LMWH) all possess significant drawbacks.

Warfarin is typically used to treat patients suffering from atrial fibrillation. The drug interacts with vitamin K-dependent coagulation factors which include factors II, VII, IX and X. Anticoagulant proteins C and S are also inhibited by warfarin. Drug therapy using warfarin is further complicated by the fact that warfarin interacts with other medications, including drugs used to treat atrial fibrillation, such as amiodarone. Because therapy with warfarin is difficult to predict, patients must be carefully monitored in order to detect any signs of anomalous bleeding.

Heparin functions by activating antithrombin which inhibits both thrombin and factor X. (Bjork I, Lindahl U. Mol Cell Biochem. 1982 48: 161-182.) Treatment with heparin may cause an immunological reaction that makes platelets aggregate within blood vessels that can lead to thrombosis. This side effect is known as heparin-induced thrombocytopenia (HIT) and requires patient monitoring. Prolonged treatment with heparin may also lead to osteoporosis. LMWH can also inhibit Factor 2, but to a lesser degree than unfractioned heparin (UFH). LMWH has been implicated in the development of HIT.

Thus, current anticoagulant agents lack predictability and specificity and, therefore, require careful patient monitoring to prevent adverse side effects, such as bleeding complications. There are currently no anticoagulants which target only the intrinsic or extrinsic pathway.

### SUMMARY OF THE INVENTION

Provided herein are antisense compounds, compositions, and methods for the treatment and prevention of clotting disorders.

Antisense compounds described herein may comprise an oligonucleotide consisting of 12 to 30 nucleosides targeted to a Factor 9 nucleic acid. In certain embodiments, the Factor 9 nucleic acid may be any of the sequences as set forth in nucleotides 22823000 to 22858000 of GENBANK Accession No. NT_{—}011786.15 (SEQ ID NO: 1), GENBANK Accession No. AB186358.1 (SEQ ID NO: 2), or GENBANK Accession No. NM_{—}000133.2 (SEQ ID NO: 3).

The antisense compound may be a single-stranded or double-stranded oligonucleotide. The antisense compound may be 100, 95, 90, 85, 80, 75, or 70% complementary to a Factor 9 nucleic acid.

The antisense oligonucleotide may be modified, wherein at least one internucleoside linkage is a modified internucleoside linkage. The internucleoside linkage may be a phosphorothioate internucleoside linkage.

The antisense oligonucleotide may be modified, wherein at least one nucleoside comprises a modified sugar. The modified sugar may be a bicyclic sugar. The modified sugar may comprise a 2′-O-methoxyethyl.

The antisense oligonucleotide may be modified, wherein at least one nucleoside comprises a modified nucleobase. The modified nucleobase may be a 5-methylcytosine.

The antisense oligonucleotide may be a 5-10-5 MOE gapmer. The antisense oligonucleotide may consist of 20 linked nucleosides.

Compositions described herein may comprise an oligonucleotide consisting of 12 to 30 linked nucleosides, targeted to a Factor 9 nucleic acid or a salt thereof and a pharmaceutically acceptable carrier or diluent.

The composition may be a single-stranded or double-stranded oligonucleotide.

Methods described herein may comprise administering to an animal a compound comprising an oligonucleotide consisting of 12 to 30 linked nucleosides targeted to a Factor 9 nucleic acid.

Administration of the compound may slow or stop coagulation. The compound may be co-administered with any of aspirin, clopidogrel, dipyridamole, heparin, lepirudin, ticlopidine, and warfarin. Administration of the compound and a second drug may be concomitant.

Administration of the compound and/or the second drug may be by parenteral administration. Parenteral administration may be any of subcutaneous or intravenous administration.

In certain embodiments, the methods described herein may also comprise identifying a human with a clotting disorder and administering to a human a therapeutically effective amount of a compound comprising an oligonucleotide consisting of 12 to 30 linked nucleosides targeted to a Factor 9 nucleic acid.

Also described is a compound comprising an antisense oligonucleotide consisting of 12 to 30 linked nucleosides that will bind within the range of nucleobases 1246 to 1359, 7515 to 7608, 7872 to 7904, 11599 to 11671, 18861 to 18896, 18905 to 18973, 21690 to 21774, 31255 to 31284, 31292 to 31373, 32065 to 32098, 32107 to 32158, 32184 to 32273, 32303 to 32399, 32434 to 32557, 32566 to 32625, 32643 to 32694, 32716 to 32778, 32807 to 32839, 32847 to 32871, 32912 to 32969, 32985 to 33029, 33230 to 33263, 33272 to 33313, 33442 to 33488, 33558 to 33582, 33801 to 33830, 33934 to 33966, or 33230 to 33830 of SEQ ID NO: 1, encoding a Factor 9 nucleic acid.

The antisense oligonucleotide may be 90, 95, or 100% complementary to SEQ ID NO: 1, encoding a Factor 9 nucleic acid. The antisense oligonucleotide may be fully complementary to SEQ ID NO: 1.

The antisense oligonucleotide may hybridize exclusively within the range of nucleobases 1246 to 1359, 7515 to 7608, 7872 to 7904, 11599 to 11671, 18861 to 18896, 18905 to 18973, 21690 to 21774, 31255 to 31284, 31292 to 31373, 32065 to 32098, 32107 to 32158, 32184 to 32273, 32303 to 32399, 32434 to 32557, 32566 to 32625, 32643 to 32694, 32716 to 32778, 32807 to 32839, 32847 to 32871, 32912 to 32969, 32985 to 33029, 33230 to 33263, 33272 to 33313, 33442 to 33488, 33558 to 33582, 33801 to 33830, 33934 to 33966, or 33230 to 33830 of SEQ ID NO: 1, encoding a Factor 9 nucleic acid.

Certain embodiments of the invention provide a compound comprising a modified oligonucleotide consisting of 12 to 30 linked nucleosides and having a nucleobase sequence, wherein the nucleobase sequence comprises an at least 12 consecutive nucleobase portion complementary to a equal number of nucleobases of nucleotides 33230 to 33313 of SEQ ID NO: 1, wherein the modified oligonucleotide is at least 80% complementary to SEQ ID NO: 1.

In certain embodiments the modified oligonucleotide comprises a nucleobase sequence of any of the group consisting of SEQ ID NO: 52, SEQ ID NO: 53, SEQ ID NO: 54, and SEQ ID NO: 118.

Certain embodiments of the invention provide a compound comprising a modified oligonucleotide consisting of 12 to 30 linked nucleosides comprising an at least 12 consecutive nucleobase portion complementary to an equal number of nucleobases of nucleotides 33230 to 33313 of SEQ ID NO: 1, wherein the modified oligonucleotide is at least 80% complementary to SEQ ID NO: 1 or a salt thereof and a pharmaceutically acceptable carrier or diluent.

Certain embodiments of the invention provide a method comprising administering to an animal a compound comprising a modified oligonucleotide consisting of 12 to 30 linked nucleosides comprising an at least 12 consecutive nucleobase portion complementary to an equal number of nucleobases of nucleotides 33230 to 33313 of SEQ ID NO: 1, wherein the modified oligonucleotide is at least 80% complementary to SEQ ID NO: 1.

Certain embodiments of the invention provide a method comprising identifying an animal at risk for thromboembolic complications and administering to the at risk animal a therapeutically effective amount of a compound comprising a modified oligonucleotide consisting of 12 to 30 linked nucleosides, wherein the modified oligonucleotide is complementary to a Factor 9 nucleic acid.

Embodiments of the present invention provide, a compound comprising a modified oligonucleotide consisting of 12 to 30 linked nucleosides comprising a nucleobase sequence comprising at least 12 contiguous nucleobases of a nucleobase sequence of SEQ ID NO: 4 to 135 and 141 to 153.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or”, unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“5-methylcytosine” means a cytosine modified with a methyl group attached to the 5′ position. A 5-methylcytosine is a modified nucleobase.

“Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Amelioration” refers to a lessening of at least one indicator, sign, or symptom of an associated disease, disorder, or condition. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antidote compound” refers to a compound capable decreasing the intensity or duration of any antisense activity.

“Antidote oligonucleotide” means an antidote compound comprising an oligonucleotide that is complementary to and capable of hybridizing with an antisense compound.

“Antidote protein” means an antidote compound comprising a peptide.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furosyl ring modified by the bridging of two non-geminal ring atoms. A bicyclic sugar is a modified sugar.

“Bicyclic nucleic acid” or “BNA” or “bicyclic nucleoside” or bicyclic nucleotide” refers to a nucleoside or nucleotide wherein the furanose portion of the nucleoside or nucleotide includes a bridge connecting two carbon atoms on the furanose ring, thereby forming a bicyclic ring system. As used herein, unless otherwise indicated, the term “methyleneoxy BNA” alone refers to β-D-methyleneoxy BNA.

“Cap structure” or “terminal cap moiety” means chemical modifications, which have been incorporated at either terminus of an antisense compound.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions, each position having a plurality of subunits.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses administration in parallel or sequentially.

“Coagulation factor” means any of factors I, II, III, IV, V, VII, VIII, IX, X, XI, XII, or XIII in the blood coagulation cascade. “Coagulation factor nucleic acid” means any nucleic acid encoding a coagulation factor. For example, in certain embodiments, a coagulation factor nucleic acid includes, without limitation, a DNA sequence encoding a coagulation factor (including genomic DNA comprising introns and exons), an RNA sequence transcribed from DNA encoding a coagulation factor, and an mRNA sequence encoding a coagulation factor. “Coagulation factor mRNA” means an mRNA encoding a coagulation factor protein.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, in drugs that are injected the diluent may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Efficacy” means the ability to produce a desired effect. “Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Factor 9 nucleic acid” or “Factor IX nucleic acid” means any nucleic acid encoding Factor 9. For example, in certain embodiments, a Factor 9 nucleic acid includes, without limitation, a DNA sequence encoding Factor 9, an RNA sequence transcribed from DNA encoding Factor 9 (including genomic DNA comprising introns and exons), and an mRNA sequence encoding Factor 9. “Factor 9 mRNA” means an mRNA encoding a Factor 9 protein.

“Factor 9 specific inhibitor” refers to any agent capable of specifically inhibiting the expression of Factor 9 mRNA and/or Factor 9 protein at the molecular level. For example, Factor 9 specific inhibitors include nucleic acids (including antisense compounds), peptides, antibodies, small molecules, and other agents capable of inhibiting the expression of Factor 9 mRNA and/or Factor 9 protein. In certain embodiments, by specifically modulating Factor 9 mRNA expression and/or Factor 9 protein expression, Factor 9 specific inhibitors may affect other components of the coagulation cascade including downstream components. Similarly, in certain embodiments, Factor 9 specific inhibitors may affect other molecular processes in an animal.

“Factor 9 specific inhibitor antidote” means a compound capable of decreasing the effect of a Factor 9 specific inhibitor. In certain embodiments, a Factor 9 specific inhibitor antidote is selected from a Factor 9 peptide; a Factor 9 antidote oligonucleotide; including a Factor 9 antidote compound complementary to a Factor 9 antisense compound; and any compound or protein that affects the intrinsic or extrinsic coagulation pathway.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid. In certain such embodiments, an antisense oligonucleotide is a first nucleic acid and a target nucleic acid is a second nucleic acid.

“Gapmer” means an antisense compound in which an internal position having a plurality of nucleotides that supports RNaseH cleavage is positioned between external regions having one or more nucleotides that are chemically distinct from the nucleosides of the internal region. A “gap segment” means the plurality of nucleotides that make up the internal region of a gapmer. A “wing segment” means the external region of a gapmer.

“Gap-widened” means a chimeric antisense compound having a gap segment of 12 or more contiguous 2′-deoxyribonucleosides positioned between and immediately adjacent to 5′ and 3′ wing segments having from one to six nucleosides.

“Hybridization” means the annealing of complementary nucleic acid molecules. In certain embodiments, complementary nucleic acid molecules include, but are not limited to, an antisense compound and a target nucleic acid. In certain such embodiments, complementary nucleic acid molecules include, but are not limited to, an antisense oligonucleotide and a nucleic acid target.

“Identifying an animal at risk for thromboembolic complications” means identifying an animal having been diagnosed with a thromboembolic complication, or identifying an animal predisposed to develop a thromboembolic complication. Individuals predisposed to develop a thromboembolic complication include those having one or more risk factors for thromboembolic complications including immobility, surgery (particularly orthopedic surgery), malignancy, pregnancy, older age, use of oral contraceptives, and inherited or acquired prothrombotic clotting disorders. Such identification may be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Individual in need thereof” refers to a human or non-human animal selected for treatment or therapy that is in need of such treatment or therapy.

“Internucleoside linkage” refers to the chemical bond between nucleosides.

“Linked nucleosides” means adjacent nucleosides which are bonded together.

“Mismatch” or “non-complementary nucleobase” means a nucleobase of a first nucleic acid that is not capable of pairing with the corresponding nucleobase of a second or target nucleic acid.

“Modified internucleoside linkage” refers to a substitution and/or any change from a naturally occurring internucleoside bond (i.e. a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising a modified internucleoside linkage, a modified sugar, and/or a modified nucleobase.

“Modified sugar” refers to a substitution and/or any change from a natural sugar. “Modified sugar moiety” means a sugar moiety having any substitution and/or change from a natural sugar moiety.

“Motif” means the pattern of unmodified and modified nucleosides in an antisense compound, i.e. the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, and/or nucleobase modification.

“Nucleoside” means a nucleobase linked to a sugar.

“Nucleotide” means a nucleoside having a phosphate group covalently linked to the sugar portion of the nucleoside.

“Oligomeric compound” or “oligomer” means a polymer comprising linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes, but is not limited to, subcutaneous administration, intravenous administration, intramuscular administration, intraarterial administration, intraperitoneal administration, or intracranial administration, e.g. intrathecal or intracerebroventricular administration. “Subcutaneous administration” means administration just below the skin. “Intravenous administration” means administration into the veins.

“Peptide” means a molecule formed by linking at least two amino acids by amide bonds. Peptide refers to polypeptides and proteins.

“Pharmaceutical agent” means a substance provides a therapeutic benefit when administered to an individual. For example, in certain embodiments, an antisense oligonucleotide targeted to Factor 9 is a pharmaceutical agent. “Active pharmaceutical agent” means the substance or substances in a pharmaceutical composition that provides a desired effect.

“Pharmaceutical composition” means a mixture of substances suitable for administering to an individual. For example, a pharmaceutical composition may comprise one or more antisense oligonucleotides and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset or development of a disease, disorder, or condition for a period of time from minutes to indefinitely. “Prevent” also means reducing risk of developing a disease, disorder, or condition.

“Prodrug” means a therapeutic agent that is prepared in an inactive form that is converted to an active form (i.e., drug) within the body or cells thereof by the action of endogenous enzymes or other chemicals or conditions.

“Side effects” means physiological responses attributable to a treatment other than the desired effects. In certain embodiments, side effects include, without limitation, injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand. “Single-stranded modified oligonucleotide” means a modified oligonucleotide which is not hybridized to a complementary strand.

“Specifically hybridizable” means an antisense compound that hybridizes to a target nucleic acid to induce a desired effect, while exhibiting minimal or no effects on non-target nucleic acids. For example, specifically hybridizable refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e. under physiological conditions in the case of in vivo assays and therapeutic treatments.

“Stringent hybridization conditions” means conditions under which a nucleic acid molecule, such as an antisense compound, will hybridize to a target nucleic acid sequence, but to a minimal number of other sequences. Stringent conditions are sequence-dependent and will vary in different circumstances. In the context of this invention, stringent conditions under which oligomeric compounds hybridize to a target sequence are determined by the nature and composition of the oligomeric compounds and the assays in which they are being investigated.

“Targeted” or “targeted to” menas having a nucleobase sequence that will allow specific hybridization of an antisense compound to a target nucleic acid to induce a desired effect. In certain embodiments, a desired effect is reduction of a target nucleic acid. In certain embodiments, a desired effect is reduction of a Factor 9 mRNA.

“Targeting” means the process of design and selection of an antisense compound that will specifically hybridize to a target nucleic acid and induce a desired effect.

“Target nucleic acid,” “target RNA,” “target RNA transcript,” and “nucleic acid target” all mean a nucleic acid capable of being targeted by antisense compounds.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Target region” or “active target region” means a portion of a target nucleic acid to which one or more antisense compounds is targeted.

“Therapeutically effective amount” means an amount of a pharmaceutical agent that provides a therapeutic benefit to an individual.

“Thromboembolic complication” means any disease, disorder, or condition involving an embolism caused by a thrombus. Examples of such diseases, disorders, and conditions include the categories of thrombosis, embolism, and thromboembolism. In certain embodiments, such disease disorders, and conditions include deep vein thrombosis, pulmonary embolism, myocardial infarction, and stroke.

“Treat” refers to administering a pharmaceutical composition to effect an alteration or improvement of a disease, disorder, or condition.

“Unmodified nucleotide” means a nucleotide composed of naturally occuring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

Embodiments of the present invention provide methods, compounds, and compositions for modulating expression of Factor 9 mRNA and protein. In certain embodiments, expression of Factor 9 mRNA and protein is decreased. In certain embodiments, Factor 9 specific inhibitors modulate expression of Factor 9 mRNA and protein. In certain embodiments, Factor 9 specific inhibitors are nucleic acids, proteins, or small molecules.

In certain embodiments, modulation can occur in a cell or tissue. In certain embodiments, the cell or tissue is in an animal. In certain embodiments, the animal is a human. In certain embodiments, Factor 9 mRNA levels are reduced. In certain embodiments, Factor 9 protein levels are reduced. Such reduction can occur in a time-dependent manner or in a dose-dependent manner.

Embodiments of the present invention provide methods, compounds, and compositions for the treatment, prevention, or amelioration of diseases, disorders, and conditions associated with Factor 9 in an individual in need thereof. In certain embodiments, such diseases, disorders, and conditions are thromboembolic complications. Such thromboembolic complications include the categories of thrombosis, embolism, and thromboembolism. In certain embodiments such thromboembolic complications include deep vein thrombosis, pulmonary embolism, myocardial infarction, and stroke.

Such diseases, disorders, and conditions can have one or more risk factors, causes, or outcomes in common. Certain risk factors and causes for development of a thromboembolic complication include immobility, surgery (particularly orthopedic surgery), malignancy, pregnancy, older age, use of oral contraceptives, atrial fibrillation, previous thromboembolic complication, chronic inflammatory disease, and inherited or acquired prothrombotic clotting disorders. Certain outcomes associated with development of a thromboembolic complication include decreased blood flow through an affected vessel, death of tissue, and death.

In certain embodiments, methods of treatment include administering a Factor 9 specific inhibitor to an individual in need thereof.

In certain embodiments, the present invention provides methods and compounds for the preparation of a medicament for the treatment, prevention, or amelioration of a disease, disorder, or condition associated with Factor 9. Factor 9 associated diseases, disorders, and conditions include thromboembolic complications such as thrombosis, embolism, thromboembolism, deep vein thrombosis, pulmonary embolism, myocardial infarction, and stroke

Embodiments of the present invention provide a Factor 9 specific inhibitor for use in treating, preventing, or ameliorating a Factor 9 associated disease. In certain embodiments, Factor 9 specific inhibitors are nucleic acids (including antisense compounds), peptides, antibodies, small molecules, and other agents capable of inhibiting the expression of Factor 9 mRNA and/or Factor 9 protein.

Embodiments of the present invention provide a Factor 9 specific inhibitor, as described herein, for use in treating, preventing, or ameliorating thromboembolic complications such as thrombosis, embolism, thromboembolism, deep vein thrombosis, pulmonary embolism, myocardial infarction, and stroke.

Embodiments of the present invention provide a Factor 9 specific inhibitor, as described herein, for use in treating, preventing, or ameliorating a thromboembolic complication, as described herein, by combination therapy with an additional agent or therapy, as described herein. Agents or therapies can be co-administered or administered concomitantly.

Embodiments of the present invention provide the use of a Factor 9 specific inhibitor, as described herein, in the manufacture of a medicament for treating, preventing, or ameliorating a thromboembolic complication, as described herein, by combination therapy with an additional agent or therapy, as described herein. Agents or therapies can be co-administered or administered concomitantly.

Embodiments of the present invention provide the use of a Factor 9 specific inhibitor, as described herein, in the manufacture of a medicament for treating, preventing, or ameliorating a thromboembolic complication, as described herein, in a patient who is subsequently administered an additional agent or therapy, as described herein.

Embodiments of the present invention provide a kit for treating, preventing, or ameliorating a thromboembolic complication, as described herein, wherein the kit comprises:
(i) a Factor 9 specific inhibitor as described herein; and alternatively
(ii) an additional agent or therapy as described herein.

A kit of the present invention may further include instructions for using the kit to treat, prevent, or ameliorate a thromboembolic complication, as described herein, by combination therapy, as described herein.

Embodiments of the present invention provide antisense compounds targeted to a Factor 9 nucleic acid. In certain embodiments, the human Factor 9 nucleic acid is any of the sequences set forth in GENBANK Accession No. NT_{—}011786.15, truncated at 22823000 to 22858000 (incorporated herein as SEQ ID NO: 1); GENBANK Accession No. AB186358.1 (incorporated herein as SEQ ID NO: 2); and GENBANK Accession No. NM_{—}000133.2 (incorporated herein as SEQ ID NO: 3).

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that it is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a Factor 9 nucleic acid is 12 to 30 subunits in length. In other words, antisense compounds are from 12 to 30 linked subunits. In other embodiments, the antisense compound is 8 to 80, 12 to 50, 15 to 30, 18 to 24, 19 to 22, or 20 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleotides.

In certain embodiments, a shortened or truncated antisense compound targeted to a Factor 9 nucleic acid has a single subunit deleted from the 5′ end (5′ truncation), or alternatively from the 3′ end (3′ truncation). A shortened or truncated antisense compound targeted to a Factor 9 nucleic acid may have two subunits deleted from the 5′ end, or alternatively may have two subunits deleted from the 3′ end, of the antisense compound. Alternatively, the deleted nucleosides may be dispersed throughout the antisense compound; for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other; for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al. (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358,1988) tested a series of tandem 14 nucleobase antisense oligonucleotides, and 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a Factor 9 nucleic acid have chemically modified subunits arranged in patterns, or motifs, to confer to the antisense compounds properties, such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer, an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment supports cleavage of the target nucleic acid, while the wing segments comprise modified nucleosides to enhance stability, affinity, and exonuclease resistance. In certain embodiments, the regions of a gapmer are differentiated by the types of sugar moieties comprising each distinct region. The types of sugar moieties that are used to differentiate the regions of a gapmer may, in some embodiments, include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n-O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30, or more nucleotides. Thus, gapmers of the present invention include, but are not limited to, for example, 5-10-5, 4-8-4, 4-12-3, 4-12-4, 3-14-3, 2-13-5, 2-16-2, 1-18-1, 3-10-3, 2-10-2, 1-10-1 or 2-8-2.

In certain embodiments, the antisense compound has a “wingmer” motif, having a wing-gap or gap-wing configuration, i.e. an X-Y or Y-Z configuration, as described above, for the gapmer configuration. Thus, wingmer configurations of the present invention include, but are not limited to, for example, 5-10, 8-4, 4-12, 12-4, 3-14, 16-2, 18-1, 10-3, 2-10, 1-10, 8-2, 2-13, or 5-13.

In certain embodiments, antisense compounds targeted to a Factor 9 nucleic acid possess a 5-10-5 gapmer motif.

In certain embodiments, antisense compounds targeted to a Factor 9 nucleic acid possess a 3-14-3 gapmer motif.

In certain embodiments, antisense compounds targeted to a Factor 9 nucleic acid possess a 2-13-5 gapmer motif.

In certain embodiments, antisense compounds targeted to a Factor 9 nucleic acid possess a 2-12-2 gapmer motif.

In certain embodiments, an antisense compound targeted to a Factor 9 nucleic acid has a gap-widened motif.

In certain embodiments, a gap-widened antisense oligonucleotide targeted to a Factor 9 nucleic acid has a gap segment of fourteen 2′-deoxyribonucleotides positioned immediately adjacent to and between wing segments of three chemically modified nucleosides. In certain embodiments, the chemical modification comprises a 2′-sugar modification. In another embodiment, the chemical modification comprises a 2′-MOE sugar modification.

In certain embodiments, a gap-widened antisense oligonucleotide targeted to a Factor 9 nucleic acid has a gap segment of thirteen 2′-deoxyribonucleotides positioned immediately adjacent to and between a 5′ wing segment of two chemically modified nucleosides and a 3′ wing segment of five chemically modified nucleosides. In certain embodiments, the chemical modification comprises a 2′-sugar modification. In another embodiment, the chemical modification comprises a 2′-MOE sugar modification.

### Target Nucleic Acids, Target Regions and Nucleotide Sequences

Nucleotide sequences that encode Factor 9 gene sequence include, without limitation, the following: GENBANK® Accession No. NT_{—}011786.15, truncated from 22823000 to 22858000, first deposited with GENBANK® on Nov. 29, 2000, incorporated herein as SEQ ID NO: 1; GENBANK Accession No. AB186358.1, first deposited with GENBANK® on Feb. 7, 2005, and incorporated herein as SEQ ID NO: 2; GENBANK® Accession No. NM_{—}000133.2, first deposited with GENBANK® on Mar. 24, 1999, incorporated herein as SEQ ID NO: 3; and GENBANK Accession No. NT_{—}039706.6, truncated from 5038000 to 5071000, first deposited with GENBANK® on Feb. 24, 2003, and incorporated herein as SEQ ID NO: 136.

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (ISIS No.) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, a translation termination region, or other defined nucleic acid regions. The structurally defined regions for Factor 9 gene sequences can be obtained by accession number from sequence databases, such as NCBI, and such information is incorporated herein by reference. In certain embodiments, a target region may encompass the sequence from a 5′ target site of one target segment within the target region to a 3′ target site of another target segment within the target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region, such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in Factor 9 mRNA levels are indicative of inhibition of Factor 9 expression. Reductions in levels of a Factor 9 protein are also indicative of inhibition of target mRNA expression. Further, phenotypic changes are indicative of inhibition of Factor 9 expression. For example, a prolonged PT time can be indicative of inhibition of Factor 9 expression. In another example, prolonged aPTT time in conjunction with a prolonged PT time can be indicative of inhibition of Factor 9 expression. In another example, a decreased level of Platelet Factor 4 (PF-4) expression can be indicative of inhibition of Factor 9 expression. In another example, reduced formation of thrombus or increased time for thrombus formation can be indicative of inhibition of Factor 9 expression.

### Hybridization

In some embodiments, hybridization occurs between an antisense compound disclosed herein and a Factor 9 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a Factor 9 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a Factor 9 nucleic acid).

Non-complementary nucleobases between an antisense compound and a Factor 9 nucleic acid may be tolerated provided that the antisense compound remains able to specifically hybridize to a target nucleic acid. Moreover, an antisense compound may hybridize over one or more segments of a Factor 9 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a Factor 9 nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an antisense compound which is 18 nucleobases in length having 4 (four) non-complementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, antisense compound may be fully complementary to a Factor 9 nucleic acid, or a target region, or a target segment or target sequence thereof As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to each nucleobase of the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a Factor 9 nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a Factor 9 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA, which contains uracil in place of thymidine in a disclosed DNA sequence, would be considered identical to the DNA sequence since both uracil and thymidine pair with adenine. Shortened and lengthened versions of the antisense compounds described herein, as well as compounds having non-identical bases relative to the antisense compounds provided herein, are also contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occuring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties, such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a Factor 9 nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity or some other beneficial biological property to the antisense compounds. In certain embodiments, nucleosides comprise chemically modified ribofuranose ring moieties. Examples of chemically modified ribofuranose rings include, without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R1)(R)2 (R═H, C1-C12 alkyl or a protecting group) and combinations thereof. Examples of chemically modified sugars include 2′-F-5′-methyl substituted nucleoside (see PCT International Application WO 2008/101157, published on Aug. 21, 2008, for other disclosed 5′,2′-bis substituted nucleosides) or replacement of the ribosyl ring oxygen atom with ‘S’ and with further substitution at the 2′-position (see U.S. Patent Application US2005-0130923, published on Jun. 16, 2005) or alternatively 5′-substitution of a BNA (see PCT International Application WO 2007/134181, published on Nov. 22, 2007, wherein LNA is substituted with, for example, a 5′-methyl or a 5′-vinyl group).

Examples of nucleosides having modified sugar moieties include, without limitation, nucleosides comprising 5′-vinyl, 5′-methyl (R or S), 4′-S, 2′-F, 2′-OCH₃ and 2′-O(CH₂)2OCH₃ substituent groups. The substituent at the 2′ position can also be selected from allyl, amino, azido, thio, O-allyl, O—C1-C10 alkyl, OCF₃, O(CH₂)2SCH₃, O(CH₂)₂—O—N(Rm)(Rn), and O—CH₂—C(═O)—N(Rm)(Rn), where each Rm and Rn is, independently, H or substituted or unsubstituted C1-C10 alkyl.

Examples of bicyclic nucleic acids (BNAs) include, without limitation, nucleosides comprising a bridge between the 4′ and the 2′ ribosyl ring atoms. In certain embodiments, antisense compounds provided herein include one or more BNA nucleosides, wherein the bridge comprises one of the formulas: 4′-(CH₂)—O-2′ (LNA); 4′-(CH₂)—S-2′; 4′-(CH₂)—O-2′ (LNA); 4′-(CH₂)₂—O-2′ (ENA); 4′-C(CH₃)₂—O-2′ (see PCT/US2008/068922); 4′-CH(CH₃) [symbol] -O-2′ and 4′-C [symbol] H(CH₂OCH₃) [symbol] -O-2′ (see U.S. Pat. No. 7,399,845, issued on Jul. 15, 2008); 4′-CH₂—N(OCH₃)-2′ (see PCT/US2008/064591); 4′-CH₂—O—N(CH₃)-2′ (see U.S. Patent Application US2004-0171570, published Sep. 2, 2004); 4′-CH₂—N(R)—O-2′ (see U.S. Pat. No. 7,427,672, issued on Sep. 23, 2008); 4′-CH₂—C(CH₃)-2′and 4′-CH₂—C [symbol] (=CH₂)-2′ (see PCT/US2008/066154); and wherein R is, independently, H, C1-C12 alkyl, or a protecting group. Each of the foregoing BNAs include various stereochemical sugar configurations, including, for example, α-L-ribofuranose and β-D-ribofuranose (see PCT international application PCT/DK98/00393, published on Mar. 25, 1999 as WO 99/14226).

In certain embodiments, nucleosides are modified by replacement of the ribosyl ring with a sugar surrogate. Such modification includes, without limitation, replacement of the ribosyl ring with a surrogate ring system (sometimes referred to as DNA analogs), such as a morpholino ring, a cyclohexenyl ring, a cyclohexyl ring or a tetrahydropyranyl ring, such as one having one of the formulas:

Many other bicyclo and tricyclo sugar surrogate ring systems are also known in the art that can be used to modify nucleosides for incorporation into antisense compounds (see, for example, review article: Leumann, Christian J.). Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds targeted to a Factor 9 nucleic acid comprise one or more nucleotides having modified sugar moieties. In certain embodiments, the modified sugar moiety is 2′-MOE. In certain embodiments, the 2′-MOE modified nucleotides are arranged in a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional unmodified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly, 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to a Factor 9 nucleic acid comprise one or more modified nucleobases. In certain embodiments, gap-widened antisense oligonucleotides targeted to a Factor 9 nucleic acid comprise one or more modified nucleobases. In certain embodiments, the modified nucleobase is 5-methylcytosine. In certain embodiments, each cytosine is a 5-methylcytosine.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compounds targeted to a Factor 9 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment employed in the methods described herein is a pharmaceutical composition comprising an antisense compound targeted to a Factor 9 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602, published on Jan. 16, 2003.

### Cell Culture and Antisense Compounds Treatment

The effects of antisense compounds on the level, activity or expression of Factor 9 nucleic acids can be tested in vitro in a variety of cell types. Cell types used for such analyses are available from commerical vendors (e.g. American Type Culture Collection, Manassus, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include HepG2 cells, HepB3 cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE® (Invitrogen, Carlsbad, Calif.). Antisense oligonucleotide is mixed with LIPOFECTAMINE® in OPTI-MEM® 1 reduced serum medium (Invitrogen, Carlsbad, Calif.) to achieve the desired concentration of antisense oligonucleotide and a LIPOFECTAMINE® concentration that typically ranges 2 to 12 ug/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTIN®. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL® Reagent (Invitrogen, Carlsbad, Calif.), according to the manufacturer's recommended protocols.

### Analysis of Inhibition of Target Levels or Expression

Inhibition of levels or expression of a Factor 9 nucleic acid can be assayed in a variety of ways known in the art. For example, target nucleic acid levels can be quantitated by, e.g., Northern blot analysis, competitive reverse transcription polymerase chain reaction (RT-PCR), or quantitative real-time RT-PCR. RNA analysis can be performed on total cellular RNA or poly(A)+ mRNA. Methods of RNA isolation are well known in the art. Northern blot analysis is also routine in the art. Real-time RT-PCR can be conveniently accomplished using the commercially available ABI PRISM® 7600, 7700, or 7900 Sequence Detection System, available from PE-Applied Biosystems, Foster City, Calif., and used according to manufacturer's instructions.

### Quantitative Real-Time RT-PCR Analysis of Target RNA Levels

Quantitation of target RNA levels may be accomplished by quantitative real-time RT-PCR using the ABI PRISM® 7600, 7700, or 7900 Sequence Detection System (PE-Applied Biosystems, Foster City, Calif.) according to manufacturer's instructions. Methods of real-time RT-PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT and real-time-PCR reactions are carried out by methods well known to those skilled in the art.

Gene (or RNA) target quantities obtained by real time RT-PCR are normalized using either the expression level of a gene whose expression is constant, such as cyclophilin A, or by quantifying total RNA using RIBOGREEN® (Invitrogen, Inc. Carlsbad, Calif.). Cyclophilin A expression is quantified by real time RT-PCR, by being run simultaneously with the target, multiplexing, or separately. Total RNA is quantified using RIBOGREEN® RNA quantification reagent (Invetrogen, Inc. Eugene, Oreg.). Methods of RNA quantification by RIBOGREEN® are taught in Jones, L. J., et al., (Analytical Biochemistry, 1998, 265, 368-374). A CYTOFLUOR® 4000 instrument (PE Applied Biosystems) is used to measure RIBOGREEN® fluorescence.

Probes and primers are designed to hybridize to a Factor 9 nucleic acid. Methods for designing real-time RT-PCR probes and primers are well known in the art, and may include the use of software such as PRIMER EXPRESS® Software (Applied Biosystems, Foster City, Calif.).

### Analysis of Protein Levels

Antisense inhibition of Factor 9 nucleic acids can be assessed by measuring Factor 9 protein levels. Protein levels of Factor 9 can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of human and mouse Factor 9 are commercially available.

### In Vivo Testing of Antisense Compounds

Antisense compounds, for example, antisense oligonucleotides, are tested in animals to assess their ability to inhibit expression of Factor 9 and produce phenotypic changes, such as, prolonged PT, prolonged aPTT time, decreased quantity of Platelet Factor 4 (PF-4), reduced formation of thrombus or increased time for thrombus formation, and reduction of cellular proliferation. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from liver tissue and changes in Factor 9 nucleic acid expression are measured. Changes in Factor 9 protein levels are also measured using a thrombin generation assay. In addition, effects on clot times, e.g. PT and aPTT, are determined using plasma from treated animals.

### Certain Indications

In certain embodiments, the invention provides methods of treating an individual comprising administering one or more pharmaceutical compositions of the present invention. In certain embodiments, the individual has a thromboembolic complication. In certain embodiments, the individual is at risk for a blood clotting disorder, including, but not limited to, infarction, thrombosis, embolism, thromboembolism, such as deep vein thrombosis, pulmonary embolism, myocardial infarction, and stroke. This includes individuals with an acquired problem, disease, or disorder that leads to a risk of thrombosis, for example, surgery, cancer, immobility, sepsis, atherosclerosis, atrial fibrillation, as well as genetic predisposition, for example, antiphospholipid syndrome and the autosomal dominant condition, Factor V Leiden. In certain embodiments, the individual has been identified as in need of anti-coagulation therapy. Examples of such individuals include, but are not limited to, those undergoing major orthopedic surgery (e.g., hip/knee replacement or hip fracture surgery) and patients in need of chronic treatment, such as those suffering from atrial fibrillation to prevent stroke. In certain embodiments the invention provides methods for prophylactically reducing Factor 9 expression in an individual. Certain embodiments include treating an individual in need thereof by administering to an individual a therapeutically effective amount of an antisense compound targeted to a Factor 9 nucleic acid.

In one embodiment, administration of a therapeutically effective amount of an antisense compound targeted to a Factor 9 nucleic acid is accompanied by monitoring of Factor 9 levels in the serum of an individual, to determine an individual's response to administration of the antisense compound. An individual's response to administration of the antisense compound is used by a physician to determine the amount and duration of therapeutic intervention.

In certain embodiments, administration of an antisense compound targeted to a Factor 9 nucleic acid results in reduction of Factor 9 expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a Factor 9 nucleic acid results in a change in a measure of blood clotting, as measured by a standard test, for example, but not limited to, activated partial thromboplastin time (aPTT) test, prothrombin time (PT) test, thrombin time (TCT), bleeding time, or D-dimer. In certain embodiments, administration of a Factor 9 antisense compound increases the measure by at least 15, 20, 25, 30, 35, 40, 45, 50, 55, 60, 65, 70, 75, 80, 85, 90, 95 or 99%, or a range defined by any two of these values. In some embodiments, administration of a Factor 9 antisense compound decreases the measure by at least 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to Factor 9 are used for the preparation of a medicament for treating a patient suffering or susceptible to a thromboembolic complication.

### Certain Combination Therapies

In certain embodiments, one or more pharmaceutical compositions of the present invention are co-administered with one or more other pharmaceutical agents. In certain embodiments, such one or more other pharmaceutical agents are designed to treat the same disease, disorder, or condition as the one or more pharmaceutical compositions of the present invention. In certain embodiments, such one or more other pharmaceutical agents are designed to treat a different disease, disorder, or condition as the one or more pharmaceutical compositions of the present invention. In certain embodiments, such one or more other pharmaceutical agents are designed to treat an undesired side effect of one or more pharmaceutical compositions of the present invention. In certain embodiments, one or more pharmaceutical compositions of the present invention are co-administered with another pharmaceutical agent to treat an undesired effect of that other pharmaceutical agent. In certain embodiments, one or more pharmaceutical compositions of the present invention are co-administered with another pharmaceutical agent to produce a combinational effect. In certain embodiments, one or more pharmaceutical compositions of the present invention are co-administered with another pharmaceutical agent to produce a synergistic effect.

In certain embodiments, one or more pharmaceutical compositions of the present invention and one or more other pharmaceutical agents are administered at the same time. In certain embodiments, one or more pharmaceutical compositions of the present invention and one or more other pharmaceutical agents are administered at different times. In certain embodiments, one or more pharmaceutical compositions of the present invention and one or more other pharmaceutical agents are prepared together in a single formulation. In certain embodiments, one or more pharmaceutical compositions of the present invention and one or more other pharmaceutical agents are prepared separately.

In certain embodiments, pharmaceutical agents that may be co-administered with a pharmaceutical composition of the present invention include anticoagulant or antiplatelet agents. In certain embodiments, pharmaceutical agents that may be co-administered with a pharmaceutical composition of the present invention include, but are not limited to aspirin, clopidogrel, dipyridamole, ticlopidine, warfarin (and related coumarins), heparin, direct thrombin inhibitors (such as lepirudin, bivalirudin), apixaban, lovenox, and small molecular compounds that interfere directly with the enzymatic action of particular coagulation factors (e.g. rivaroxaban, which interferes with Factor Xa). In certain embodiments, the anticoagulant or antiplatelet agent is administered prior to administration of a pharmaceutical composition of the present invention. In certain embodiments, the anticoagulant or antiplatelet agent is administered following administration of a pharmaceutical composition of the present invention. In certain embodiments the anticoagulant or antiplatelet agent is administered at the same time as a pharmaceutical composition of the present invention. In certain embodiments the dose of a co-administered anticoagulant or antiplatelet agent is the same as the dose that would be administered if the anticoagulant or antiplatelet agent was administered alone. In certain embodiments the dose of a co-administered anticoagulant or antiplatelet agent is lower than the dose that would be administered if the anticoagulant or antiplatelet agent was administered alone. In certain embodiments the dose of a co-administered anticoagulant or antiplatelet agent is greater than the dose that would be administered if the anticoagulant or antiplatelet agent was administered alone.

In certain embodiments, the co-administration of a second compound enhances the anticoagulant effect of a first compound, such that co-administration of the compounds results in an anticoagulant effect that is greater than the effect of administering the first compound alone. In other embodiments, the co-administration results in anticoagulant effects that are additive of the effects of the compounds when administered alone. In certain embodiments, the co-administration results in anticoagulant effects that are supra-additive of the effects of the compounds when administered alone. In certain embodiments, the first compound is an antisense compound. In certain embodiments, the second compound is an antisense compound.

In certain embodiments, an antidote is administered anytime after the administration of a Factor 9 specific inhibitor. In certain embodiments, an antidote is administered anytime after the administration of an antisense oligonucleotide targeting Factor 9. In certain embodiments, the antidote is administered minutes, hours, days, weeks, or months after the administration of an antisense compound targeting Factor 9. In certain embodiments, the antidote is a complementary (e.g. a sense strand) to the antisense compound targeting Factor 9. In certain embodiments, the antidote is a Factor 9 or Factor 9a protein. In certain embodiments, the Factor 9 or Factor 9a protein is a human Factor 9 or human Factor 9a protein.

### Examples

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Inhibition of Human Factor 9: Primary Cynomolgus Hepatocytes

Antisense oligonucleotides targeted to a Factor 9 nucleic acid were tested for their effects on Factor 9 mRNA in vitro. Primary Cynomolgus hepatocytes at a density of 35,000 cells per well in a 96-well plate were treated with 150 nM of antisense oligonucleotide. After a treatment period of approximately 16 hours, RNA was isolated from the cells and Factor 9 mRNA levels were measured by quantitative real-time PCR, as described herein. Factor 9 primer probe set ABI F9 was used to measure mRNA levels. Factor 9 mRNA levels were adjusted according to total RNA content as measured by RIBOGREEN®. Results are presented as percent inhibition of Factor 9, relative to untreated control cells in Table 1.

The antisense oligonucleotides were designed as 5-10-5 gapmers, where the gap segment comprises 2′-deoxynucleotides and each wing segment comprises 2′-MOE nucleotides. “5′ target site” indicates the 5′-most nucleotide which the antisense oligonucleotide is targeted to SEQ ID NO: 1 (nucleotides 22823000 to 22858000 of GENBANK Accession No. NT_{—}011786.15) or SEQ ID NO: 2 (GENBANK Accession No. AB186358.1).

TABLE 1
Inhibition of human Factor 9 mRNA levels by chimeric
oligonucleotides having 5-10-5 MOE wings and deoxy gap

| | Target | Target | Target | | | SEQ |
|---|---|---|---|---|---|---|
| Oligo | SEQ | Start | Stop | | % | ID |
| ID | ID NO | Site | Site | Sequence (5′ to 3′) | Inhibition | NO |
| 402585 | 1 | 31310 | 31329 | ACAGTGGGCAGCAGTTACAA | 0 | 4 |
| 402610 | 1 | 32508 | 32527 | GTATATATTCCATATTTGCC | 24 | 5 |
| 407670 | 1 | 1283 | 1302 | TTCTGCCATGATCATGTTCA | 25 | 6 |
| 407671 | 1 | 1313 | 1332 | TAAAAGGCAGATGGTGATGA | 8 | 7 |
| 407672 | 1 | 1323 | 1342 | GTAGATATCCTAAAAGGCAG | 8 | 8 |
| 407673 | 1 | 1333 | 1352 | TCAGCACTGAGTAGATATCC | 24 | 9 |
| 407674 | 1 | 7515 | 7534 | TTCATGATCAAGAAAAACTG | 4 | 10 |
| 407675 | 1 | 7538 | 7557 | CGATTCAGAATTTTGTTGGC | 29 | 11 |
| 407676 | 1 | 7548 | 7567 | CCTCTTTGGCCGATTCAGAA | 19 | 12 |
| 407679 | 1 | 7589 | 7608 | AGGTTCCCTTGAACAAACTC | 0 | 13 |
| 407681 | 1 | 11599 | 11618 | ATTTAAACATGGATTGGACT | 7 | 14 |
| 407682 | 1 | 11631 | 11650 | TAGGAATTAATGTCATCCTT | 16 | 15 |
| 407683 | 1 | 11646 | 11665 | GGACACCAACATTCATAGGA | 15 | 16 |
| 407684 | 1 | 18869 | 18888 | TAATGTTACATGTTACATCT | 26 | 17 |
| 407686 | 1 | 18933 | 18952 | GTACAGGAGCAAACCACCTT | 7 | 18 |
| 407687 | 1 | 18954 | 18973 | TCTGCAAGTCGATATCCCTC | 9 | 19 |
| 407697 | 1 | 21690 | 21709 | TGATTGGGTGCTTTGAGTGA | 21 | 20 |
| 407698 | 1 | 21700 | 21719 | AGTCATTAAATGATTGGGTG | 0 | 21 |
| 407699 | 1 | 21710 | 21729 | ACCCGAGTGAAGTCATTAAA | 15 | 22 |
| 407700 | 1 | 21740 | 21759 | TGACCTGGTTTGGCATCTTC | 31 | 23 |
| 407701 | 1 | 21755 | 21774 | ACCTGCCAAGGGAATTGACC | 4 | 24 |
| 407702 | 1 | 31255 | 31274 | GCATCAACTTTACCATTCAA | 19 | 25 |
| 407703 | 1 | 31265 | 31284 | TCCACAGAATGCATCAACTT | 17 | 26 |
| 407704 | 1 | 31320 | 31339 | CAGTTTCAACACAGTGGGCA | 1 | 27 |
| 407706 | 1 | 32116 | 32135 | ATGGTTGTACTTATTAATAG | 7 | 28 |
| 407708 | 1 | 32139 | 32158 | TCCAGTTCCAGAAGGGCAAT | 28 | 29 |
| 407709 | 1 | 32200 | 32219 | CGTGTATTCCTTGTCAGCAA | 36 | 30 |
| 407710 | 1 | 32232 | 32251 | ACATAGCCAGATCCAAATTT | 0 | 31 |
| 407711 | 1 | 32254 | 32273 | GAAGACTCTTCCCCAGCCAC | 2 | 32 |
| 407712 | 1 | 32303 | 32322 | CAAGTGGAACTCTAAGGTAC | 23 | 33 |
| 407713 | 1 | 32313 | 32332 | GCTCGGTCAACAAGTGGAAC | 5 | 34 |
| 407714 | 1 | 32323 | 32342 | AAGACATGTGGCTCGGTCAA | 9 | 35 |
| 407715 | 1 | 32343 | 32362 | ATGGTGAACTTTGTAGATCG | 12 | 36 |
| 407716 | 1 | 32434 | 32453 | TTCCACTTCAGTAACATGGG | 14 | 37 |
| 407717 | 1 | 32448 | 32467 | AAGAAACTGGTCCCTTCCAC | 16 | 38 |
| 407718 | 1 | 32458 | 32477 | AATTCCAGTTAAGAAACTGG | 5 | 39 |
| 407719 | 1 | 32483 | 32502 | TTGCACACTCTTCACCCCAG | 38 | 40 |
| 407720 | 1 | 32495 | 32514 | ATTTGCCTTTCATTGCACAC | 14 | 41 |
| 407721 | 1 | 32528 | 32547 | TGACATACCGGGATACCTTG | 20 | 42 |
| 407722 | 1 | 32576 | 32595 | GGAAATCCATCTTTCATTAA | 8 | 43 |
| 407723 | 1 | 32586 | 32605 | AATTAACCTTGGAAATCCAT | 37 | 44 |
| 407724 | 1 | 32667 | 32686 | TAGAATGTATATATTCAAAT | 0 | 45 |
| 407725 | 1 | 32736 | 32755 | CCATTTTCTAATCAATTTGC | 32 | 46 |
| 407726 | 1 | 32759 | 32778 | CACATTATATTCCTCTAGTG | 48 | 47 |
| 407727 | 1 | 32912 | 32931 | GAAGATCGGGAAGATGGAAT | 4 | 48 |
| 407728 | 1 | 32922 | 32941 | GAGAAGCAAAGAAGATCGGG | 0 | 49 |
| 407730 | 1 | 32942 | 32961 | AAACATTGATGTTTTGGTTG | 20 | 50 |
| 407731 | 1 | 32985 | 33004 | TGATAGAGTAGACCAAAGAT | 6 | 51 |
| 407733 | 1 | 33230 | 33249 | TTGATAGACATGTATAACTG | 45 | 52 |
| 407734 | 1 | 33244 | 33263 | AAGCAAGTCTGGGTTTGATA | 36 | 53 |
| 407735 | 1 | 33272 | 33291 | TGTTCTGAAAAGCAAGTCTC | 64 | 54 |
| 407737 | 1 | 33442 | 33461 | GCTTCCATTATATGTGTGTA | 53 | 55 |
| 407738 | 1 | 33654 | 33673 | TTGAATTCTTCCTCAAAGTC | 33 | 56 |
| 407739 | 1 | 33801 | 33820 | GCTGATTGGAATGACTTATG | 47 | 57 |
| 407741 | 1 | 33857 | 33876 | ACTATAATCAAAATGTTCCA | 0 | 58 |
| 407743 | 1 | 33937 | 33956 | ACACCAGTTTATTAATTCAC | 45 | 59 |
| 407744 | 1 | 33947 | 33966 | ATGAACCAGAACACCAGTTT | 40 | 60 |
| 407745 | 1 | 7872 | 7891 | CATACTGCTTCCAAAATTCA | 19 | 61 |
| 407705 | 2 | 713 | 732 | ATGTTCACCTGCGACAACTG | 37 | 62 |

### Example 2

### Antisense Inhibition of Human Factor 9 mRNA in Primary Cynomolgus Hepatocytes

Antisense oligonucleotides targeted to a Factor 9 nucleic acid were tested for their effects on Factor 9 mRNA in vitro. Primary Cynomolgus hepatocytes at a density of 35,000 cells per well in a 96-well plate were treated with 250 nM of antisense oligonucleotide. After a treatment period of approximately 16 hours, RNA was isolated from the cells and Factor 9 mRNA levels were measured by quantitative real-time RT-PCR, as described herein. Factor 9 primer probe set ABI F9 was used to measure mRNA levels. Factor 9 mRNA levels were adjusted according to total RNA content as measured by RIBOGREEN®. Results are presented as percent inhibition of Factor 9, relative to untreated control cells in Table 2.

The antisense oligonucleotides were designed as 5-10-5 gapmers, where the gap segment comprises 2′-deoxynucleotides and each wing segment comprises 2′-MOE nucleotides. “5′ target site” indicates the 5′-most nucleotide which the antisense oligonucleotide is targeted to SEQ ID NO: 1 (nucleotides 22823000 to 22858000 of GENBANK Accession No. NT_{—}011786.15) or SEQ ID NO: 3 (GENBANK Accession No. NM_{—}000133.2).

TABLE 2
Inhibition of human Factor 9 mRNA levels by chimeric
oligonucleotides having 5-10-5 MOE wings and deoxy gap

| | Target | Target | Target | | | |
|---|---|---|---|---|---|---|
| Oligo | SEQ | Start | Stop | | % | SEQ |
| ID | ID NO | Site | Site | Sequence (5′ to 3′) | Inhibition | ID NO |
| 402603 | 1 | 32363 | 32382 | CACAGAACATGTTGTTATAG | 3 | 63 |
| 402633 | 1 | 7885 | 7904 | AATTGCTTACCAACATACTG | 0 | 64 |
| 407749 | 1 | 1293 | 1312 | GGCCTGGTGATTCTGCCATG | 34 | 65 |
| 407750 | 1 | 1303 | 1322 | ATGGTGATGAGGCCTGGTGA | 13 | 66 |
| 407751 | 1 | 1340 | 1359 | TGTACATTCAGCACTGAGTA | 19 | 67 |
| 407752 | 1 | 7557 | 7576 | TGAATTATACCTCTTTGGCC | 15 | 68 |
| 407756 | 1 | 11638 | 11657 | ACATTCATAGGAATTAATGT | 0 | 69 |
| 407759 | 1 | 18877 | 18896 | GCCATTCTTAATGTTACATG | 12 | 70 |
| 407761 | 1 | 18923 | 18942 | AAACCACCTTGTTATCAGCA | 33 | 71 |
| 407767 | 1 | 21624 | 21643 | ATCAGGAAAAACAGTCTCAG | 12 | 72 |
| 407768 | 1 | 21660 | 21679 | AATGGTTTCAGCTTCAGTAG | 0 | 73 |
| 407769 | 1 | 21720 | 21739 | TCCACCAACAACCCGAGTGA | 8 | 74 |
| 407773 | 1 | 32065 | 32084 | AATCACATTTCGCTTTTGCT | 0 | 75 |
| 407774 | 1 | 32079 | 32098 | TGAGGAATAATTCGAATCAC | 30 | 76 |
| 407776 | 1 | 32184 | 32203 | GCAATGCAAATAGGTGTAAC | 4 | 77 |
| 407777 | 1 | 32218 | 32237 | AAATTTGAGGAAGATGTTCG | 0 | 78 |
| 407779 | 1 | 32333 | 32352 | TTGTAGATCGAAGACATGTG | 20 | 79 |
| 407780 | 1 | 32353 | 32372 | GTTGTTATAGATGGTGAACT | 12 | 80 |
| 407781 | 1 | 32373 | 32392 | TGGAAGCCAGCACAGAACAT | 0 | 81 |
| 407782 | 1 | 32380 | 32399 | TCCTTCATGGAAGCCAGCAC | 28 | 82 |
| 407783 | 1 | 32468 | 32487 | CCCAGCTAATAATTCCAGTT | 32 | 83 |
| 407784 | 1 | 32518 | 32537 | GGATACCTTGGTATATATTC | 2 | 84 |
| 407785 | 1 | 32538 | 32557 | TTAATCCAGTTGACATACCG | 27 | 85 |
| 407786 | 1 | 32566 | 32585 | CTTTCATTAAGTGAGCTTTG | 30 | 86 |
| 407792 | 1 | 32807 | 32826 | ACAATTTTGTCAAGGGCTGG | 7 | 87 |
| 407795 | 1 | 32852 | 32871 | TGGAGAACCATAGTATCTGA | 24 | 88 |
| 407796 | 1 | 32950 | 32969 | GAACTAATAAACATTGATGT | 0 | 89 |
| 407798 | 1 | 33010 | 33029 | CTTCATGAGTGTGGTACTGG | 64 | 90 |
| 407799 | 1 | 33202 | 33221 | AGTATAACAGAATGATGCTC | 44 | 91 |
| 407803 | 1 | 33469 | 33488 | CCATACAAGCTCTTAGAATG | 52 | 92 |
| 407809 | 1 | 33811 | 33830 | AACTTAGTTGGCTGATTGGA | 42 | 93 |
| 407812 | 1 | 6376 | 6395 | GTAAGCTATACCATTTAGGT | 0 | 94 |
| 407813 | 1 | 7230 | 7249 | AAGAAAGTTCTCAAATGAGC | 0 | 95 |
| 407814 | 1 | 11688 | 11707 | AGTTACTTACCTAATTCACA | 10 | 96 |
| 407815 | 1 | 18861 | 18880 | CATGTTACATCTAAAAGAAG | 16 | 97 |
| 402583 | 1 | 31298 | 31317 | AGTTACAATCCATTTTTCAT | 0 | 98 |
| 407746 | 1 | 1246 | 1265 | GCTAGCAGATTGTGAAAGTG | 0 | 99 |
| 407747 | 1 | 1264 | 1283 | ACGCGCTGCATAACCTTTGC | 9 | 100 |
| 407748 | 1 | 1270 | 1289 | ATGTTCACGCGCTGCATAAC | 0 | 101 |
| 407754 | 1 | 11609 | 11628 | AACTGCCGCCATTTAAACAT | 0 | 102 |
| 407755 | 1 | 11624 | 11643 | TAATGTCATCCTTGCAACTG | 0 | 103 |
| 407757 | 1 | 11652 | 11671 | CCAAAGGGACACCAACATTC | 0 | 104 |
| 407760 | 1 | 18905 | 18924 | CACTATTTTTACAAAACTGC | 0 | 105 |
| 407771 | 1 | 31292 | 31311 | AATCCATTTTTCATTAACGA | 0 | 106 |
| 407772 | 1 | 31331 | 31350 | AATTTTAACACCAGTTTCAA | 0 | 107 |
| 407775 | 1 | 32107 | 32126 | CTTATTAATAGCTGCATTGT | 4 | 108 |
| 407778 | 1 | 32243 | 32262 | CCCAGCCACTTACATAGCCA | 0 | 109 |
| 407787 | 1 | 32596 | 32615 | AATTCCAATGAATTAACCTT | 0 | 110 |
| 407788 | 1 | 32606 | 32625 | GTTAATTTTCAATTCCAATG | 0 | 111 |
| 407789 | 1 | 32643 | 32662 | CAAAAGATGGGAAAGTGATT | 5 | 112 |
| 407790 | 1 | 32675 | 32694 | AATGATCATAGAATGTATAT | 0 | 113 |
| 407791 | 1 | 32716 | 32735 | TCAGGTAAAATATGAAATTC | 0 | 114 |
| 407793 | 1 | 32820 | 32839 | AGAATTTAACTTCACAATTT | 0 | 115 |
| 407794 | 1 | 32847 | 32866 | AACCATAGTATCTGATGGAC | 20 | 116 |
| 407797 | 1 | 33002 | 33021 | GTGTGGTACTGGCCTTGTGA | 32 | 117 |
| 407801 | 1 | 33294 | 33313 | CAGGCACCTTACTTCATCCC | 41 | 118 |
| 407802 | 1 | 33455 | 33474 | AGAATGGCTTATTGCTTCCA | 51 | 119 |
| 407804 | 1 | 33528 | 33547 | AGTTACAATGATATGCCAAT | 1 | 120 |
| 407805 | 1 | 33558 | 33577 | CAATATGTCTGGGTCAATGT | 1 | 121 |
| 407806 | 1 | 33563 | 33582 | GAGTACAATATGTCTGGGTC | 0 | 122 |
| 407807 | 1 | 33623 | 33642 | TAGATTGCAAACGAACGGTT | 5 | 123 |
| 407808 | 1 | 33795 | 33814 | TGGAATGACTTATGCCTTTG | 16 | 124 |
| 407810 | 1 | 33902 | 33921 | TCAGTTGGTCAGCAACTCTC | 50 | 125 |
| 407811 | 1 | 33934 | 33953 | CCAGTTTATTAATTCACAAA | 0 | 126 |
| 407816 | 1 | 20104 | 20123 | TTAAGGACATGTTCCTGCTG | 0 | 127 |
| 407817 | 1 | 24462 | 24481 | CTAAATTGAGCCTTTATACC | 0 | 128 |
| 407818 | 1 | 30267 | 30286 | ATTAAAGTGCACACAGTCTT | 0 | 129 |
| 407819 | 1 | 31128 | 31147 | GGTGCTGGCAGAAATGGAAT | 6 | 130 |
| 407820 | 1 | 31354 | 31373 | GTGTATTTACCTGCGACAAC | 0 | 131 |
| 407753 | 3 | 288 | 307 | TCAACATACTGCTTCCAAAA | 22 | 132 |
| 407758 | 3 | 409 | 428 | TGTTACATCTAATTCACAGT | 21 | 133 |
| 407770 | 3 | 742 | 761 | CAAAACAACCTGCCAAGGGA | 10 | 134 |
| 407800 | 3 | 2086 | 2105 | CTATGGAAGCAAGTCTGGGT | 28 | 135 |

### Example 3

### Antisense Inhibition of Human Factor 9 mRNA in Primary Cynomolgus Hepatocytes

Antisense oligonucleotides targeted to a Factor 9 nucleic acid were tested for their effects on Factor 9 mRNA in vitro. Primary Cynomolgus hepatocytes at a density of 35,000 cells per well in a 96-well plate were treated with 150 nM of antisense oligonucleotide. After a treatment period of approximately 16 hours, RNA was isolated from the cells and Factor 9 mRNA levels were measured by quantitative real-time PCR, as described herein. Factor 9 primer probe set ABI F9 was used to measure mRNA levels. Factor 9 mRNA levels were adjusted according to total RNA content as measured by RIBOGREEN®. Results are presented as percent inhibition of Factor 9, relative to untreated control cells in Table 3.

The antisense oligonucleotides were designed as 5-10-5 gapmers, where the gap segment comprises 2′-deoxynucleotides and each wing segment comprises 2′-MOE nucleotides. “5′ target site” indicates the 5′-most nucleotide which the antisense oligonucleotide is targeted to SEQ ID NO: 1 (nucleotides 22823000 to 22858000 of GENBANK Accession No. NT_{—}011786.15).

TABLE 3
Inhibition of human Factor 9 mRNA levels by chimeric
oligonucleotides having 5-10-5 MOE wings and deoxy gap

| | Target | Target | Target | | | |
|---|---|---|---|---|---|---|
| Oligo | SEQ | Start | Stop | | % | SEQ |
| ID | ID NO | Site | Site | Sequence (5′ to 3′) | Inhibition | ID NO |
| 407677 | 1 | 7567 | 7586 | CCAATTTACCTGAATTATAC | 1 | 141 |
| 407678 | 1 | 7579 | 7598 | GAACAAACTCTTCCAATTTA | 2 | 142 |
| 407680 | 1 | 11589 | 11608 | GGATTGGACTCACACTGATC | 0 | 143 |
| 407685 | 1 | 18883 | 18902 | GCATCTGCCATTCTTAATGT | 2 | 144 |
| 407694 | 1 | 21650 | 21669 | GCTTCAGTAGAATTTACATA | 0 | 145 |
| 407695 | 1 | 21670 | 21689 | TGTTATCCAAAATGGTTTCA | 7 | 146 |
| 407696 | 1 | 21680 | 21699 | CTTTGAGTGATGTTATCCAA | 0 | 147 |
| 407707 | 1 | 32126 | 32145 | GGGCAATGTCATGGTTGTAC | 0 | 148 |
| 407729 | 1 | 32932 | 32951 | GTTTTGGTTGGAGAAGCAAA | 5 | 149 |
| 407732 | 1 | 33046 | 33065 | ATGAGTTTTAGCCTCTCAGC | 11 | 150 |
| 407736 | 1 | 33282 | 33301 | TTCATCCCTATGTTCTGAAA | 0 | 151 |
| 407740 | 1 | 33821 | 33840 | AGAAAAGGACAACTTAGTTG | 0 | 152 |
| 407742 | 1 | 33868 | 33887 | TAGAAGGATTAACTATAATC | 4 | 153 |

### Example 4

### Antisense Inhibition of Human Factor 9 in Primary Cynomolgus Hepatocytes

Several antisense oligonucleotides exhibiting in vitro inhibition of Factor 9 were tested at various doses in primary Cynomolgus hepatocytes. Cells were plated at densities of 35,000 cells per well and treated with nM concentrations of antisense oligonucleotide as indicated in Table 4. After a treatment period of approximately 16 hours, RNA was isolated from the cells and Factor 9 mRNA levels were measured by quantitative real-time RT-PCR, as described herein. Human Factor 9 primer probe set ABI F9 was used to measure mRNA levels. Factor 9 mRNA levels were adjusted according to total RNA content as measured by RIBOGREEN®. Results are presented as percent inhibition of Factor 9, relative to untreated control cells. As illustrated in Table 4, Factor 9 mRNA levels were reduced in a dose-dependent manner.

TABLE 4
Antisense Inhibition of human Factor 9 in primary Cynomolgus
hepatocytes, Primer Probe Set
ABI F9

| ISIS | | | | | | |
|---|---|---|---|---|---|---|
| No | 9.375 nM | 18.75 nM | 37.5 nM | 75 nM | 150 nM | 300 nM |
| 407735 | 0 | 9 | 36 | 60 | 86 | 97 |
| 407737 | 0 | 0 | 7 | 29 | 54 | 66 |
| 407726 | 0 | 0 | 0 | 19 | 44 | 64 |
| 407739 | 0 | 0 | 0 | 36 | 58 | 80 |
| 407682 | 0 | 0 | 0 | 9 | 35 | 42 |
| 407734 | 2 | 0 | 0 | 9 | 26 | 57 |
| 407798 | 0 | 0 | 6 | 35 | 63 | 80 |
| 407802 | 0 | 1 | 0 | 19 | 48 | 64 |
| 407801 | 0 | 0 | 0 | 6 | 13 | 39 |
| 407776 | 0 | 0 | 0 | 0 | 18 | 20 |

### Example 5

### Antisense Inhibition of Murine Factor 9 In Vitro

Chimeric antisense oligonucleotides having 5-10-5 MOE wings and deoxy gap were designed to target murine Factor 9 (nucleotides 5038000 to 5071000 of GENBANK Accession No. NT_{—}039706.6), incorporated herein as SEQ ID NO: 136. The gapmers are 20 nucleotides in length, wherein the central gap segment is comprised of 10 2′-deoxynucleotides and is flanked on both sides (in the 5′ and 3′ directions) by wings comprising 5 nucleotides each. Each nucleotide in each wing segment has a 2′-MOE modification. The internucleoside linkages throughout each gapmer are phosphorothioate (P═S) linkages. All cytidine residues throughout each gapmer are 5′-methylcytidines. The antisense oligonucleotides were evaluated for their ability to reduce Factor 9 mRNA in primary mouse hepatocytes.

Primary mouse hepatocytes were treated with 4.6875 nM, 9.375 nM, 18.75 nM, 37.5 nM, 75.0 nM, and 150.0 nM of antisense oligonucleotides for a period of approximately 24 hours. RNA was isolated from the cells and Factor 9 mRNA levels were measured by quantitative real-time RT-PCR, as described herein. Murine Factor 9 primer probe set RTS 2845 (forward sequence: TCCTGCACTGAGGGATACCAA, incorporated herein as SEQ ID NO: 138; reverse sequence: TCCCACATGGAAATGGAACTG, incorporated herein as SEQ ID NO: 139; probe sequence: TTGCAGAAGACCAGAAGTCCTGTGAACCAX, incorporated herein as SEQ ID NO: 140) was used to measure mRNA levels. Factor 9 mRNA levels were adjusted according to total RNA content as measured by RIBOGREEN®. Several of the antisense oligonucleotides reduced Factor 9 mRNA levels in a dose-dependent manner

### Example 6

### Antisense Inhibition of Murine Factor 9 In Vivo

Four antisense oligonucleotides showing statistically significant dose-dependent inhibition from the in vitro study (see Example 4) were evaluated for their ability to reduce Factor 9 mRNA in vivo. Target start sites for the four antisense oligonucleotides are as follows: 920, 31017, 31164, and 31183.

### Treatment

ISIS 402618 (GGAGAATTGGGTACATCTCT; target site 31183), incorporated herein as SEQ ID NO: 137, was evaluated in a group of 4 male Balb/c mice 8 weeks of age and compared to a control group treated with saline. Oligonucleotide or saline was administered subcutaneously at a dose of 5 mg/kg, 10 mg/kg, 25 mg/kg, or 50 mg/kg twice a week for three weeks. After the treatment period, plasma was collected for clotting analysis (PT/aPTT) and whole liver was collected for RNA and protein analysis. Treatment with ISIS 402618 resulted in statistically significant dose-dependent reduction of Factor 9 mRNA and Factor 9 protein in comparison to the control sample (saline).

### RNA Analysis

Liver RNA was isolated for real-time PCR analysis of Factor 9. Treatment with ISIS 402618 at a dose of 5 mg/kg resulted in a 73% reduction of Factor 9. Treatment with ISIS 402618 at a dose of 10 mg/kg resulted in an 85% reduction of Factor 9. Treatment with ISIS 402618 at a dose of 25 mg/kg resulted in a 95% reduction of Factor 9. Treatment with ISIS 402618 at a dose of 50 mg/kg resulted in a 98% reduction of Factor 9. Results are shown in Table 5.

TABLE 5
Dose-dependent antisense inhibition of murine
Factor 9 mRNA in BALB/c mice

| mg/kg | % inhibition |
|---|---|
| 5 | 73 |
| 10 | 85 |
| 25 | 95 |
| 50 | 98 |

### Protein Analysis

Plasma Factor 9 protein was measured using a Factor 9 chromogenic assay (Hyphen BioMed). Treatment with ISIS 402618 at a dose of 5 mg/kg resulted in a 72% reduction of Factor 9. Treatment with ISIS 402618 at a dose of 10 mg/kg resulted in an 83% reduction of Factor 9. Treatment with ISIS 402618 at a dose of 25 mg/kg resulted in a 97% reduction of Factor 9. Treatment with ISIS 402618 at a dose of 50 mg/kg resulted in a 99% reduction of Factor 9. Results are shown in Table 6.

TABLE 6
Dose-dependent antisense inhibition of murine
Factor 9 protein in BALB/c mice

| mg/kg | % inhibition |
|---|---|
| 5 | 72 |
| 10 | 83 |
| 25 | 97 |
| 50 | 99 |

Clotting Analysis (PT and aPTT Assay)

Prothrombin Time (PT) and Activated Partial Thromboplastin Time (aPTT) were measured using platelet poor plasma (PPP) from mice treated with ISIS 402618. PT and aPTT values provided in Table 7 are reported as International Normalized Ratio (INR) values. INR values for PT and aPTT were determined by dividing the PT or aPTT value for the experimental group by the PT or aPTT for the PBS treated group. This ratio was then raised to the power of the International Sensitivity Index (ISI) of the tissue factor used.

There was no statistical change in PT for all dosages. aPTT was significantly prolonged and correlated to the degree of target inhibition in a dose-dependent manner. aPTT was prolonged 28% at 5 mg/kg, aPTT was prolonged 27% at 10 mg/kg, aPTT was prolonged 45% at 25 mg/kg, and aPTT was prolonged 55% at 50 mg/kg.

As shown in Table 7, aPTT was significantly prolonged in mice treated with ISIS 402618 compared to the control. However, PT was not significantly prolonged in mice treated with ISIS 402618 at these doses as compared to the control. These data suggest that ISIS 402618 affects the intrinsic pathway of blood coagulation.

TABLE 7
Effect of ISIS 402618 on PT and aPTT in BALB/c mice

| mg/kg | PT INR | aPTT INR |
|---|---|---|
| 5 | 1.00 | 1.28 |
| 10 | 0.99 | 1.27 |
| 25 | 0.99 | 1.45 |
| 50 | 0.99 | 1.55 |

### Example 7

### Single Dose Pharmacokinetic Assay of ISIS 402618

The half-life and duration of action of ISIS 402618 in mice was evaluated. A group of 27 BALB/c mice was injected with 50 mg/kg of ISIS 402618. Three mice were sacrificed on days 1, 2, 3, 4, 6, 8, 12, 24, and 56 after the single dose of ISIS 402618 was administered. A control group of 3 mice was injected with a single dose of PBS, and mice in this group were sacrificed on day 1. Mice in all groups were sacrificed by cervical dislocation following anesthesia with 150 mg/kg ketamine mixed with 10 mg/kg xylazine administered by intraperitoneal injection. Liver was harvested for RNA analysis and plasma was collected for clotting analysis (PT and aPTT).

### RNA Analysis

RNA was extracted from liver tissue for quantitative real-time RT-PCR analysis of Factor 9. Results are presented as percent inhibition of Factor 9, relative to PBS control. As shown in Table 8, treatment with ISIS 402618 resulted in 89% inhibition of murine Factor 9 mRNA by day 6 after which the effect of ISIS 402618 decreased to 73% by day 12. These data show that the peak effect of a single dose of 50 mg/kg of ISIS 402618 occurs on about day 6 and duration of action

TABLE 8
Antisense inhibition of murine Factor 9 mRNA in BALB/c
mice in a single dose pharmacokinetic study

| | % |
|---|---|
| Days | inhibition |
| 1 | 51 |
| 2 | 84 |
| 3 | 86 |
| 4 | 87 |
| 6 | 89 |
| 8 | 84 |
| 12 | 73 |

PT and aPTT Assay

Prothrombin Time (PT) and Activated Partial Thromboplastin Time (aPTT) were measured using platelet poor plasma (PPP) from mice treated with ISIS 402618. PT and aPTT values provided in Table 9 are reported as International Normalized Ratio (INR) values. INR values for PT and aPTT were determined by dividing the PT or aPTT value for the experimental group (i.e. 50 mg/kg treatment with ISIS 402618) by the PT or aPTT for the PBS treated group. This ratio was then raised to the power of the International Sensitivity Index (ISI) of the tissue factor used.

As shown in Table 9, PT was not significantly different in ISIS 402618 treated mice in comparison to PBS treated mice. aPTT increased from 1.03 to 1.32 on day 6. aPTT decreased gradually after day 6 until aPTT reached 1.02 on day 56. Consistent with the mRNA expression data (above), these data show that the peak effect of a single dose of 50 mg/kg of ISIS 402618 occurs on about day 6 and duration of action lasts at least 24 days.

TABLE 9
PT and aPTT analysis in BALB/c mice in a single
dose pharmacokinetic study

| Days | PT INR | aPTT INR |
|---|---|---|
| 1 | 1.01 | 1.03 |
| 2 | 1.00 | 1.18 |
| 3 | 1.00 | 1.21 |
| 4 | 1.04 | 1.17 |
| 6 | 1.02 | 1.32 |
| 8 | 1.04 | 1.25 |
| 12 | 1.02 | 1.14 |
| 24 | 1.03 | 1.07 |
| 56 | 1.02 | 1.02 |

### Example 8

### Multiple Dose Pharmacokinetic Assay of ISIS 402618

### Treatment

The duration of action of multiple doses of ISIS 402618 on antisense inhibition of murine factor 9 as well as on clotting time was evaluated. In a first group of mice, 25 mg/kg of ISIS 402618 was injected subcutaneously as a single dose Mice from the first group were sacrificed on days 1 and 3 after the single dose. In a second group of mice, 25 mg/kg of ISIS 402618 was administered subcutaneously twice a week for 1 week. Mice from the second group were sacrificed on day 3 after the last dose of ISIS 402618 was administered. In a third group of mice, 25 mg/kg of ISIS 402618 was administered subcutaneously twice a week for 2 weeks. Mice from the third group were sacrificed on day 3 after the last dose of ISIS 402618 was administered. In a fourth group of mice, 25 mg/kg of ISIS 402618 was administered subcutaneously twice a week for 3 weeks. Mice from the fourth group were sacrificed on days 3, 7, 14, 28, 42, and 56 after the last dose of ISIS 402618 was administered. A control group of 3 mice was injected with PBS in a single dose. Mice from the control group were sacrificed 1 day later. Mice in all groups were sacrificed by cervical dislocation following anesthesia with 150 mg/kg ketamine mixed with 10 mg/kg xylazine administered by intraperitoneal injection. Liver was harvested for RNA analysis and plasma was collected for clotting analysis (PT and aPTT) for mice in all groups.

### RNA Analysis

RNA was extracted from liver tissue for quantitative real-time RT-PCR analysis of Factor 9. Results are presented as percent inhibition of Factor 9, relative to PBS control. As shown in Table 10, a single dose treatment of ISIS 402618 resulted in inhibition of Factor 9 mRNA as early as day 1. Inhibition increased through day 3 in the single dose treatment group. Two doses of ISIS 402618 resulted in increased inhibition on day 3 as compared to one dose of ISIS 402618.

Six doses of ISIS 402618 resulted in increased inhibition on day 3 as compared to 6 doses of ISIS 402618 on day 7. In mice treated with 6 doses of ISIS 402618, Factor 9 inhibition declined progressively on days 7, 14, 28, 42, and 56.

TABLE 10
Dose-dependent reduction of Factor 9 mRNA in a
multiple dose pharmacokinetic study

| No. of | | % |
|---|---|---|
| doses | Day | inhibition |
| 1 | 1 | 47 |
| 1 | 3 | 77 |
| 2 | 3 | 91 |
| 4 | 3 | 95 |
| 6 | 3 | 95 |
| 6 | 7 | 93 |
| 6 | 14 | 90 |
| 6 | 28 | 58 |
| 6 | 42 | 37 |
| 6 | 56 | 28 |

PT and aPTT Assay

PPT and aPTT were measured using platelet poor plasma (PPP) from mice treated with ISIS 402618. PT and aPTT values provided in Table 11 are reported as INR values. INR values for PT and aPTT were determined by dividing the PT or aPTT value for the experimental group (i.e. 50 mg/kg treatment with ISIS 402618) by the PT or aPTT for the PBS treated group. This ratio was then raised to the power of the ISI of the tissue factor used.

As shown in Table 11, PT was slightly increased in all dosage groups treated with ISIS 402618 on testing days 3, 7, 14, 28, 42, and 56 in comparison to PBS treated animals. aPTT was increased in all dosage groups on all testing days (i.e. 1, 3, 7, 28, 42, and 56) in comparison to PBS treated animals.

TABLE 11
PT and aPTT analysis in BALB/c mice in a multiple
dose pharmacokinetic study

| No. of | | | |
|---|---|---|---|
| doses | Day | PT INR | aPTT INR |
| 1 | 1 | 1.00 | 1.11 |
| 1 | 3 | 1.01 | 1.26 |
| 2 | 3 | 1.02 | 1.46 |
| 4 | 3 | 1.02 | 1.48 |
| 6 | 3 | 1.03 | 1.38 |
| 6 | 7 | 1.06 | 1.60 |
| 6 | 14 | 1.05 | 1.33 |
| 6 | 28 | 1.05 | 1.11 |
| 6 | 42 | 1.08 | 1.08 |
| 6 | 56 | 1.05 | 1.02 |

### Example 9

### Effect of Antisense Inhibition of Murine Factor 9 with ISIS 402618 in the FeCl₃ Induced Venous Thrombosis (VT) Model Compared to Warfarin

### Treatment

ISIS 402618 and warfarin (Coumadin®) were evaluated in the FeCl₃ induced VT mouse model. In a first cohort of BALB/c mice, 50 mg/kg of ISIS 402618 was administered subcutaneously twice a week for 3 weeks. Two days after receiving the last dose of ISIS 402618, mice were sacrificed. In a second cohort of BALB/c mice, 3.6 mg/kg of warfarin was administered intraperioneally daily for 6 days. Four hours after the last dose of warfarin, mice were sacrificed. A control group of BALB/c mice was treated with PBS, administered subcutaneously twice a week for 3 weeks. Two days after the last dose of PBS, mice were sacrificed. Mice in all groups were sacrificed by cervical dislocation following anesthesia with 150 mg/kg ketamine mixed with 10 mg/kg xylazine administered by intraperitoneal injection. Thrombus formation was induced with FeCl₃ in all groups of mice.

Thrombus formation was induced by applying a piece of filter paper (2×4 mm) pre-saturated with 10% FeCl₃ solution directly on the vena cava. After 3 minutes of exposure, the filter paper was removed. Thirty minutes after the filter paper application, the vein containing the thrombus was dissected out and the clot was removed in PBS.

### Scoring of Thrombus Formation

The presence or absence of a clot in the vena cava after FeCl₃ treatment was noted. Results are presented as a percentage of mice in which clot formation was observed. As shown in Table 12, treatment with ISIS 402618 resulted in lack of clot formation. Treatment with warfarin resulted in clot formation in 60% of the mice, whereas 100% of mice in the control PBS group developed clots after FeCl₃ treatment. These data indicate that ISIS 402618 is useful for inhibiting thrombus and clot formation.

TABLE 12
Analysis of thrombus formation in the FeCl₃ induced
venous thrombosis model

| | Mice with |
|---|---|
| Treatment | clot formation (%) |
| PBS | 100 |
| Warfarin | 60 |
| ISIS 402618 | 0 |

1-29. (canceled)

30. A compound comprising an oligonucleotide consisting of 12 to 30 linked nucleosides targeted to a Factor 9 nucleic acid, wherein the oligonucleotide has a sequence that is least 90% complementary to SEQ ID NO: 1 as measured over the entirety of said oligonucleotide.

31. The compound of claim 30, wherein the oligonucleotide has a sequence that is 100% complementary to SEQ ID NO: 1 as measured over the entirety of said oligonucleotide.

32. The compound of claim 30, wherein the oligonucleotide comprises at least one modified internucleoside linkage.

33. The compound of claim 32, wherein the modified internucleoside linkage is a phosphorothioate linkage.

34. The compound of claim 30, wherein the oligonucleotide comprises at least one modified sugar.

35. The compound of claim 34, wherein the modified sugar is a 2′-O-methoxyethyl sugar or a bicyclic sugar.

36. The compound of claim 30, wherein the oligonucleotide comprises at least one modified nucleobase.

37. The compound of claim 36, wherein the modified nucleobase is a 5-methylcytosine.

38. The compound of claim 30, wherein the oligonucleotide comprises:
a gap segment consisting of linked deoxynucleosides;
a 5′ wing segment consisting of linked nucleosides;
a 3′ wing segment consisting of linked nucleosides;
wherein the gap segment is positioned immediately adjacent to and between the 5′ wing segment and the 3′ wing segment and wherein each nucleoside of each wing segment comprises a modified sugar.

39. The compound of claim 30, wherein the oligonucleotide comprises:
a gap segment consisting of ten linked deoxynucleosides;
a 5′ wing segment consisting of five linked nucleosides;
a 3′ wing segment consisting of five linked nucleosides;
wherein the gap segment is positioned immediately adjacent to and between the 5′ wing segment and the 3′ wing segment, wherein each nucleoside of each wing segment comprises a 2′-O-methoxyethyl sugar, wherein each cytosine in said oligonucleotide is a 5-methylcytosine, and wherein each internucleoside linkage of said oligonucleotide is a phosphorothioate linkage.

40. The compound of claim 39, wherein the oligonucleotide consists of 20 linked nucleosides.

41. A composition comprising the compound of claim 30 and a pharmaceutically acceptable carrier or diluent.

42. The composition of claim 41, wherein the composition further comprises a therapeutic agent selected from the group consisting of aspirin, clopidogrel, dipyridamole, heparin, lepirudin, ticlopidine, warfarin, apixaban, rivaroxaban, and lovenox.

43. A method comprising identifying an animal having a disease or condition associated with Factor 9 and administering to the animal a therapeutically effective amount of the compound of claim 30 so that expression of Factor 9 is inhibited.

44. The method of claim 43, wherein the disease or condition is a thromboembolic complication.

45. The method of claim 44, wherein the thromboembolic complication is selected from the group consisting of thrombosis, embolism, thromboembolism, deep vein thrombosis, pulmonary embolism, myocardial infarction, and stroke.

46. The method of claim 43, wherein the animal is a human.

47. The method of claim 43, comprising co-administering the compound and a therapeutic agent selected from the group consisting of aspirin, clopidogrel, dipyridamole, heparin, lepirudin, ticlopidine, warfarin, apixaban, rivaroxaban, and lovenox.

48. The method of claim 47, wherein the compound and the therapeutic agent are administered concomitantly.
